(19) United States
(12) Patent Application Publication
(10) Pub. No.: US 2026/0174780 A1
(43) Pub. Date: Jun. 25, 2026

(54) BISMUTH-THIOL COMPOSITIONS AND METHODS FOR TREATING INFLAMMATORY DISEASES

(71) Applicant: Microbion Corporation, Bozeman, MT (US)

(72) Inventor: Brett Hugh James BAKER, Boxeman, MT (US)

(21) Appl. No.: 19/129,717

(22) PCT Filed: Nov. 15, 2023
(86) PCT No.: PCT/US2023/079905

§ 371 Date: May 14, 2025

Related U.S. Application Data
(60) Provisional application No. 63/425,653, filed on Nov. 15, 2022.
(60) Provisional application No. 63/535,898, filed on Aug. 31, 2023.

(51) Int. Cl. A61K 31/555; A61K 9/16; A61P 11/00; A61P 17/06; A61P 37/06

(52) U.S. Cl. CPC A61K 31/555; A61K 9/1682; A61P 11/00; A61P 17/06; A61P 37/06

(57) ABSTRACT

The present disclosure relates to pravibismane compositions and their use in methods for treating inflammation-related diseases or conditions, such as an inflammatory disease of the lung, psoriasis, and acne vulgaris in a subject. Treatment of inflammation-related diseases and conditions according to the disclosed methods provides an increase in the expression of anti-inflammatory cytokines, including IL-10, and/or a decrease in the expression of one or more pro-inflammatory cytokines, such as one or more of IFN-γ, MIP-3a, MIP-1β, TNF-a, IL-22, IL-17A, IL-15, and IL-13.

### CROSS-REFERENCE TO RELATED APPLICATIONS

The present application claims the benefit of U.S. Provisional Application No. 63/425,653, filed Nov. 15, 2022, and U.S. Provisional Application No. 63/535,898, filed Aug. 31, 2023, each of which is incorporated herein by reference in its entirety.

### BACKGROUND

Inflammation is a necessary biological response to harmful stimuli such as wounds and infection and is subject to a complex of balancing and counter-balancing regulatory processes in the body, involving the immune (cellular and humoral, innate and adaptive), endocrine, and neural systems, and a host of other biochemical and physiological mechanisms. In normal, healthy circumstances, inflammation and related fibrotic changes are temporary adaptations to injury, ultimately resolving in a coordinated manner. However, in a large and varied group of diseases (e.g., metabolic, autoimmune, immunocompromised, infectious, neoplastic, cardiovascular diseases), environmental factors (e.g., temperature, allergens, nutritional status, medications), physical activity (e.g. strenuous, extended etc.), psychological state, age, genetic and microbiome factors, regulation of inflammatory and immune responses can be disturbed, often leading to unchecked inflammation and/or fibrosis that can seriously impair the normal functioning of affected tissues and organs.

In such cases, anti-inflammatory cytokines that promote immune homeostasis by suppressing inflammatory cytokine production by innate and adaptive immune cells and by promoting regulatory T cell (T_{reg}) function, may be generally under-expressed while pro-inflammatory cytokines that regulate activation, differentiation, and growth of immune cells may be generally over-expressed resulting in severe or prolonged inflammation. The influence of specific cytokines and the combined influence of often pleiotropic cytokines can favor the development and activity of specific subsets of immune cells, again, favoring either prolonged or severe inflammation, or alternatively, resolution of inflammation. As such, there is not a single predefined comprehensive and exacting cytokine profile that is universally expressed in different disease conditions, or even in health, especially considering the range of different responses in different people. However, the combined influence of any given cytokine and cellular profile will generally result in a pro-inflammatory state, a balanced immune function state, or an anti-inflammatory (resolution of inflammation) state.

Given the powerful immunomodulatory effect of many cytokines (pro-inflammatory, anti-inflammatory, or a pleiotropic combination) and the consequences of impaired function in inflammatory and fibrotic diseases and related experimental disease models, there have been focused efforts to ascertain the therapeutic potential of immunomodulatory compounds to address exuberant or prolonged inflammation and/or fibrosis in these diseases. In many cases, administration of non-steroidal, or even steroidal, anti-inflammatory compounds/drugs has resulted in limited efficacy in clinical settings.

Consequently, administration of a non-steroidal anti-inflammatory compound/drug, as described herein, with broad anti-inflammatory activity that, with respect to diseases characterized by exuberant or prolonged inflammation and/or fibrosis, reduces such unhealthy inflammation and/or fibrosis and promotes effective recovery from injury even considering the extremely wide spectrum of specific immune cell/cytokine inflammatory/fibrotic profiles involved in such diseases, would represent an important advancement in the treatment of inflammatory and fibrotic conditions.

Pravibismane is a bismuth-thiol compound (also known as: 1,2-(Bis(1,3-dithia-2-bismolane)thio)ethane or BisEDT):

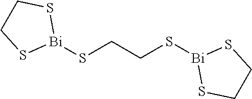

Pravibismane is known to exert pleiotropic anti-microbial effects (see WO2021195236, WO2012021754, WO2020028561, WO2020028558, WO2021195236, WO2022072842, and WO2023133588). Pravibismane has been described as being effective to treat sepsis, on the basis of a murine sepsis model involving cecal ligation and perforation (see WO2009154819 and WO1999039707). Pravibismane is understood to act as a microbial bioenergetic inhibitor, disrupting energy flow in bacterial membranes, causing the inhibition of bacterial ATP production, thereby inhibiting downstream biosynthetic activity (Baker B. 2020. 1289. Pravibismane is a Potent, Broad Spectrum Anti-Infective Small Molecule that Rapidly Disrupts Bacterial Bioenergetics and Halts Bacterial Growth. Open Forum Infect Dis 7:S659-S660).

### SUMMARY

In some embodiments, the present disclosure provides methods of treating an inflammation-related and/or fibrosis-related disease or condition in a subject in need thereof, comprising administering to the subject an effective amount of a bismuth-thiol (BT) composition comprising pravibismane.

In some embodiments, the inflammation-related disease or condition is related to depressed expression of one or more anti-inflammatory cytokines.

Accordingly, in some embodiments, the present disclosure provides methods of treating an inflammation-related and/or fibrosis-related disease or condition characterized by depressed anti-inflammatory cytokine (such as IL-10) expression, and/or increased pro-inflammatory cytokine expression (such as TNF-α), comprising administering to a subject in need thereof an effective amount of a BT composition comprising pravibismane.

In some embodiments, the present disclosure provides methods of treating an inflammation- and/or fibrosis-related disease or condition disclosed herein by modulating the production or expression of one or more cytokines in a subject in need thereof, comprising administering to the subject an effective amount of a BT composition comprising pravibismane.

In some embodiments, modulating the production or expression of one or more cytokines comprises increasing the production or expression of one or more anti-inflammatory cytokines and/or decreasing the production or expression of one or more pro-inflammatory cytokines. In some embodiments, modulating the production or expression of one or more cytokines comprises increasing the production or expression of one or more anti-inflammatory cytokines. In some embodiments, modulating the production or expression of a cytokine comprises decreasing the production or expression or one or more pro-inflammatory cytokines.

In some embodiments, the present disclosure also provides methods of treating an inflammation- or fibrosis-related disease or condition disclosed herein by increasing production or expression of one or more anti-inflammatory cytokines in a subject in need thereof, comprising administering to the subject an effective amount of a BT composition comprising pravibismane.

In some embodiments, the one or more anti-inflammatory cytokines disclosed herein is IL-6 or IL-10. In some embodiments, the anti-inflammatory cytokine is IL-10.

In some embodiments, the one or more pro-inflammatory cytokines disclosed herein is IFN-γ, MIP-3α, MIP-1β, TNF-α, IL-22, IL-17A, IL-15, and IL-13. In some embodiments, the one or more pro-inflammatory cytokines disclosed herein is IFN-γ, MIP-3α, MIP-1β, TNF-α, and IL-22. In another embodiment, the pro-fibrosis cytokine is TGFβ.

In some embodiments, the present methods of treating an inflammation-related and/or fibrosis-related disease or condition in a subject in need thereof provide a decrease in secretion of IFN-γ, MIP-3α, MIP-1β, TNF-α, IL-22, IL-17A, IL-15, and/or IL-13 compared to an untreated subject. In some embodiments, the present methods of treating an inflammation-related disease or condition in a subject in need thereof provide a decrease in secretion of IFN-γ, MIP-3α, MIP-1β, TNF-α, and/or IL-22. In some embodiments, the present methods of treating an inflammation-related disease or condition in a subject in need thereof provide a decrease in secretion of MIP-3α, MIP-1β, and/or TNF-α compared to an untreated subject. In some embodiments, secretion of MIP-3α, MIP-1β, and/or TNF-α is decreased about 2- to 10-fold compared to an untreated subject. In some embodiments, secretion of MIP-3α, MIP-1β, and/or TNF-α is decreased about 3-fold compared to an untreated subject.

In some embodiments, the present methods of treating an inflammation-related disease or condition in a subject in need thereof induce secretion of IL-2 compared to an untreated subject. In some embodiments, IL-2 section is induced about 2- to 10-fold compared to an untreated subject. In some embodiments, IL-2 section is induced about 3-fold compared to an untreated subject.

In some embodiments, the present disclosure also provides methods of treating an inflammation-related disease or condition by increasing IL-10 production or expression in a subject in need thereof, comprising administering to the subject an effective amount of pravibismane or an effective amount of a BT composition comprising pravibismane.

In some embodiments, the present disclosure provides methods of treating an inflammation-related and/or fibrosis-related disease or condition in a subject exhibiting a low level of IL-10 expression compared to a subject not afflicted with an inflammation-related disease or condition, comprising administering to the subject an effective amount of pravibismane or an effective amount of a BT composition comprising pravibismane.

In some embodiments, the present disclosure provides methods for increasing IL-10 production in a subject suffering from an inflammation-related disease or condition characterized by depressed IL-10 expression, comprising administering to the subject an effective amount of pravibismane or an effective amount of a BT composition comprising pravibismane.

In some embodiments of the disclosed methods, IL-10 production in the subject in need of treatment is increased by about 5%, about 10%, about 15%, about 20%, about 25%, about 30%, about 35%, about 40%, about 45%, about 50%, about 55%, about 60%, about 65%, about 70%, or about 75% compared to a subject not treated with pravibismane.

In some embodiments of the disclosed methods, pro-inflammatory cytokine expression in the subject in need of treatment is decreased by about 5%, about 10%, about 15%, about 20%, about 25%, about 30%, about 35%, about 40%, about 45%, about 50%, about 55%, about 60%, about 65%, about 70%, or about 75% compared to a subject not treated with pravibismane.

In some embodiments, the present disclosure provides methods of treating an inflammation-related and/or fibrosis-related disease or condition responsive to an increased level of IL-10 or an increased expression of IL-10, comprising administering to a subject diagnosed with an inflammation-related disease or condition an effective amount of pravibismane or an effective amount of a BT composition comprising pravibismane.

In some embodiments, the present disclosure provides a method for increasing the level of IL-10 expression in a cell, comprising contacting the cell with pravibismane or an effective amount of a BT composition comprising pravibismane.

In some embodiments of the disclosed methods, IL-10 expression in the cell is increased about 5%, about 10%, about 15%, about 20%, about 25%, about 30%, about 35%, about 400, about 45%, about 50%, about 55%, about 60%, about 65%, about 70%, or about 75% compared to a cell not contacted with pravibismane.

In some embodiments of the disclosed methods, pro-inflammatory cytokine expression in the cell is decreased by about 5%, about 10%, about 15%, about 20%, about 25%, about 30%, about 35%, about 40%, about 45%, about 50%, about 55%, about 60%, about 65%, about 70%, or about 75% compared to a cell not contacted with pravibismane.

In some embodiments, pravibismane may be formulated in a composition that comprises a plurality of particles or microparticles of pravibismane suspended therein, wherein the particles or microparticles have a D90 of less than about 5 μm, less than about 4 μm, less than about 3 μm, or less than about 2 μm. In some embodiments, the particles or microparticles have a D90 of less than about 2 μm. In some embodiments, the particles or microparticles have a D90 of less than about 1 μm.

In some embodiments, the inflammatory disease or condition is an inflammation-related and/or fibrosis-related disease or condition of the skin, epithelium, or of the lung. In some embodiments, the inflammation-related disease or condition is an inflammatory and/or fibrotic disease of other organ systems.

In some embodiments, the methods of the present invention include a method of treating or reducing inflammation and/or fibrosis, or an inflammation- and/or fibrosis-related disease or condition in a subject in need thereof, comprising administering to the subject an effective amount of an effective amount of a BT composition comprising pravibismane, wherein the inflammation, fibrosis, or inflammation-related disease or condition is regulated by or associated with the activation of an inflammasome. In another embodiment, the inflammasome is the NLRP3 inflammasome or the AIM2 inflammasome.

### BRIEF DESCRIPTION OF THE DRAWINGS

FIG. 1 provides a graph of IL-10 expression in A549 cells treated with BisEDT or other antibiotics.

FIG. 2A shows AIM2 expression levels relative to beta-actin expression in THP-1 macrophage cells under the following conditions: (i) uninfected, (ii) uninfected+range of BisEDT (BIZ), (iii) untreated+infected with M. avium (104), (iv) M. avium infected+4 μg/ml BIZ, (v) M. avium infected+2 μg/ml BIZ, (vi) untreated+M. abscessus (MAB), (vii) M. abscessus infected+1 μg/ml BIZ, and (viii)M. abscessus infected+0.5 μg/ml BIZ.

FIG. 2B shows NLRP3 expression levels relative to beta-actin expression in THP-1 macrophage cells under the following conditions: (i) uninfected, (ii) uninfected+range of BisEDT (BTZ), (iii) untreated+infected with M. avium (104), (iv) M. avium infected+4 μg/ml BIZ, (v) M. avium infected+2 μg/ml BIZ, (vi) M. avium infected+pre-4 μg/ml BIZ, (vii) M. avium infected+pre-2 μg/ml BIZ, (viii) untreated+M. abscessus, (ix) M. abscessus infected+1 μg/ml BIZ, (x) M. abscessus infected+0.5 μg/ml BIZ, (xi) M. abscessus infected+pre-1 μg/ml BIZ, and (xii) M. abscessus infected+pre-0.5 μg/ml BIZ.

FIG. 3 provides a schematic diagram summarizing the study groups and dose schedule for an in vivo chronic M. abscessus infection study in SCID mice. Treatment groups consisted of baseline (to determine initial infection levels), vehicle, inhaled BisEDT (200 μg/kg/day), inhaled BisEDT (1000 μg/kg/day) or inhaled amikacin (100 mg/kg/day). Treatment lasted for 28 days (6 days/week) after which the lung samples were harvested for CFU determination or histopathology.

FIG. 4 shows the colony forming units (CFU) recovered from lung homogenates in an in vivo chronic M. abscessus infection study in SCID mice.

FIGS. 5A-5D provide micrographs of mouse lung tissue: (A) infected with M. abscessus; (B) infected with M. abscessus and treated with amikacin (100 mg/kg/day); (C) infected with M. abscessus and treated with low-dose (200 s/kg/day) BisEDT; and (D) infected with M. abscessus and treated with high-dose (1000 μ/kg/day) BisEDT.

FIG. 6 provides a description of the assay for in vitro modeling of neutrophil recruitment to the lung.

FIG. 7 provides a summary of the process for comparing cellular and soluble inflammatory markers in vitro.

FIG. 8 provides a series of graphs showing that BisEDT inhibits neutrophil recruitment in the assay described in FIG. 6.

FIG. 9 provides a series of graphs quantifying cytokine levels in transmigration supernatant in the assay described in FIG. 6.

FIG. 10 provides a series of graphs quantifying cytokine levels in transmigration supernatant in the assay described in FIG. 6.

FIG. 11 provides a series of graphs quantifying IL-1 family, CSF family, and neutrophil chemoattractant cytokine levels in conditioned media in the assay described in FIG. 6 and FIG. 7.

FIG. 12 provides a series of graphs quantifying monocyte-related, macrophage-related, and anti-inflammatory cytokine levels in conditioned media in the assay described in FIG. 7 and FIG. 7.

FIG. 13A provides an experimental workflow for the ex vivo psoriasis study using BisEDT administered topically (BisEDT=pravibismane).

FIG. 13B provides an overview of the study design for evaluating the anti-inflammatory effects of BisEDT in an ex vivo human skin model of psoriasis.

FIG. 13C is a graph showing IL-22 release in HypoSkin® untreated control, HypoInflammaSkin® untreated control, and betamethasone-treated HypoInflammaSkin® positive control.

FIG. 13D is a graph showing IL-17A concentration at day 3 in HypoInflammaSkin® treated with 2.5 mg/mL topical BisEDT suspension starting on day 0 (treatment group 4) compared to untreated and positive (betamethasone) controls.

FIG. 13E is a graph showing IL-17A concentration at day 7 in HypoInflammaSkin® A treated with 2.5 mg/mL topical BisEDT suspension starting on day 0 (treatment group 4) or day 3 (treatment group 5) compared to untreated and positive (betamethasone) controls.

FIG. 13F is a graph showing TNF-α concentration at day 3 in HypoInflammaSkin® 9 treated with 2.5 mg/mL topical BisEDT suspension starting on day 0 (treatment group 4) compared to untreated and positive (betamethasone) controls.

FIG. 13G is a graph showing TNF-α concentration at day 7 in HypoInflammaSkin® treated with 2.5 mg/mL topical BisEDT suspension starting on day 0 (treatment group 4) compared to untreated and positive (betamethasone) controls.

FIG. 13H is a graph showing INFγ concentration at day 3 in HypoInflammaSkin® treated with 2.5 mg/mL topical BisEDT suspension starting on day 0 (treatment group 4) compared to untreated and positive (betamethasone) controls.

FIG. 13I is a graph showing INFγ concentration at day 7 in HypoInflammaSkin® treated with 2.5 mg/ml topical BisEDT suspension starting on day 0 (treatment group 4) or day 3 (treatment group 5) compared to untreated and positive (betamethasone) controls.

FIG. 13J is a graph showing IL-2 concentration at day 3 in HypoInflammaSkin® treated with 2.5 mg/mL topical BisEDT suspension starting on day 0 (treatment group 4) compared to untreated and positive (betamethasone) controls.

FIG. 13K is a graph showing IL-2 concentration at day 7 in HypoInflammaSkin® treated with 2.5 mg/mL topical BisEDT suspension starting on day 0 (treatment group 4) or day 3 (treatment group 5) compared to untreated and positive (betamethasone) controls.

FIG. 13L is a graph showing MIP-1β concentration at day 3 in HypoInflammaSkin® treated with 2.5 mg/mL topical BisEDT suspension starting on day 0 (treatment group 4) compared to untreated and positive (betamethasone) controls.

FIG. 13M is a graph showing MIP-1β concentration at day 7 in HypoInflammaSkin® treated with 2.5 mg/mL topical BisEDT suspension starting on day 0 (treatment group 4) or day 3 (treatment group 5) compared to untreated and positive (betamethasone) controls.

FIG. 13N is a graph showing IL-13 concentration at day 3 in HypoInflammaSkin® treated with 2.5 mg/mL topical BisEDT suspension starting on day 0 (treatment group 4) compared to untreated and positive (betamethasone) controls.

FIG. 13O is a graph showing IL-13 concentration at day 7 in HypoInflammaSkin® treated with 2.5 mg/mL topical BisEDT suspension starting on day 0 (treatment group 4) or day 3 (treatment group 5) compared to untreated and positive (betamethasone) controls.

FIG. 13P is a graph showing IL-15 concentration at day 3 in HypoInflammaSkin® treated with 2.5 mg/mL topical BisEDT suspension starting on day 0 (treatment group 4) compared to untreated and positive (betamethasone) controls.

FIG. 13Q is a graph showing IL-15 concentration at day 7 in HypoInflammaSkin® treated with 2.5 mg/mL topical BisEDT suspension starting on day 0 (treatment group 4) or day 3 (treatment group 5) compared to untreated and positive (betamethasone) controls.

FIG. 13R is a graph showing IL-22 concentration at day 7 in HypoInflammaSkin® treated with 2.5 mg/mL topical BisEDT suspension starting on day 0 (treatment group 4) or day 3 (treatment group 5) compared to untreated and positive (betamethasone) controls.

FIG. 13S is a graph showing MIP-3α concentration at day 7 in HypoInflammaSkin® treated with 2.5 mg/mL topical BisEDT suspension starting on day 0 (treatment group 4) or day 3 (treatment group 5) compared to untreated and positive (betamethasone) controls.

FIG. 14A is a graph showing IL-1β concentration in bronchoalveolar lavage fluid (BALF) samples from rats exposed to a single low dose (1 mg/kg) or single high dose (7 mg/kg) of aerosolized BisEDT. Samples 1, 2, and 3 refer to the different groups of rats (N=6) randomly assigned to a study group based on body weight.

FIG. 14B is a graph showing IL-17A concentration in bronchoalveolar lavage fluid (BALF) samples from rats exposed to a single low dose (1 mg/kg) or single high dose (7 mg/kg) of aerosolized BisEDT. Samples 1, 2, and 3 refer to the different groups of rats (N=6) randomly assigned to a study group based on body weight.

FIG. 14C is a graph showing TNF-α concentration in bronchoalveolar lavage fluid (BALF) samples from rats exposed to a single low dose (1 mg/kg) or single high dose (7 mg/kg) of aerosolized BisEDT. Samples 1, 2, and 3 refer to the different groups of rats (N=6) randomly assigned to a study group based on body weight.

FIG. 14D is a graph showing TGF-β concentration in bronchoalveolar lavage fluid (BALF) samples from rats exposed to single high dose (7 mg/kg) of aerosolized BisEDT. Samples 1, 2, and 3 refer to the different groups of rats (N=6) randomly assigned to a study group based on body weight.

FIG. 15A is a graph showing IL-10 concentration in BEAS 2B cells treated with either 4 μg/mL, 2 μg/mL, 1 μg/mL, or 0.5 μg/mL BisEDT compared to control (untreated cells), and incubated at 37° C. under 5% CO₂ conditions for 24 hours.

FIG. 15B is a graph showing TNF-α concentration in BEAS 2B cells treated with either 4 μg/mL, 2 μg/mL, 1 μg/mL, or 0.5 μg/mL BisEDT compared to control (untreated cells), and incubated at 37° C. under 5% CO₂ conditions for 24 hours.

FIG. 15C is a graph showing IL-10 concentration in BEAS 2B cells treated with either 4 μg/mL, 2 μg/mL, 1 μg/mL, or 0.5 μg/mL BisEDT compared to control (untreated cells), and incubated at 37° C. under 5% CO₂ conditions for 24 hours.

FIG. 15D is a graph showing AIM2/Actin copy number in BEAS 2B cells treated with either 4 μg/mL, 2 μg/mL, 1 μg/mL, or 0.5 μg/mL BisEDT compared to control (untreated cells), and incubated at 37° C. under 5% CO₂ conditions for 24 hours.

FIG. 16A is a graph showing IL-18 concentration in HUVEC cells treated with either 4 μg/mL, 2 μg/mL, 1 μg/mL, or 0.5 μg/mL BisEDT compared to control (untreated cells), and incubated at 37° C. under 5% CO₂ conditions for 24 hours.

FIG. 16B is a graph showing TNF-α concentration in HUVEC cells treated with either 4 μg/mL, 2 μg/mL, 1 μg/mL, or 0.5 μg/mL BisEDT compared to control (untreated cells), and incubated at 37° C. under 5% CO₂ conditions for 24 hours.

FIG. 16C is a graph showing AIM2/Actin copy number in HUVEC cells treated with either 4 μg/mL, 2 μg/mL, 1 μg/mL, or 0.5 μg/mL BisEDT compared to control (untreated cells), and incubated at 37° C. under 5% CO₂ conditions for 24 hours.

FIG. 17 is a graph showing NLRP3/Actin copy number in A549 cells treated with either 4 μg/mL, 2 μg/mL, 1 μg/mL, or 0.5 μg/mL BisEDT compared to control (untreated cells), and incubated at 37° C. under 5% CO₂ conditions for 24 hours.

FIG. 18 provides a study design for evaluating the effect of BisEDT in a fluorescein isothiocyanate (FITC)-induced acute contact hypersensitivity model.

FIG. 19 provides a graph showing the reduction in net ear-swelling compared to control BALB/c mice topically administered a topical suspension of BisEDT according to the treatment schedule in FIG. 18.

FIG. 20 provides a graph of the total histological score for treatment groups 1-5 calculated from the sum of the inflammation, necrosis, hyperplasia, and hyperkeratosis scores in Table 9.

FIG. 21A provides a graph comparing the IL-18 concentration in harvested right ears of BALB/c mice administered a topical suspension of BisEDT vs. controls and vehicle.

FIG. 21B provides a graph comparing the IL-6 concentration in harvested right ears of BALB/c mice administered a topical suspension of BisEDT vs. controls and vehicle.

FIG. 21C provides a graph comparing the IL-10 concentration in harvested right ears of BALB/c mice administered a topical suspension of BisEDT vs. controls and vehicle.

FIG. 22 provides a graph showing the reduction of TGF-β expression in M. avium-infected mice treated with BisEDT (pravibismane).

### DETAILED DESCRIPTION

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the verb “comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

Unless specifically stated or obvious from context, as used herein, the term “or” is understood to be inclusive. Unless specifically stated or obvious from context, as used herein, the terms “a”, “an”, and “the” are understood to be singular or plural.

Throughout the present specification, the terms “about” and/or “approximately” may be used in conjunction with numerical values and/or ranges. The term “about” when immediately preceding a stated number encompasses a range of +10/a of the stated number. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. These ranges comprise all subranges therein. Thus, a range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “bismuth” refers to the 83^{rd} element of the periodic table, or atoms or ions thereof. Bismuth can occur in the metallic state or in the ionized state, such as in the III or V oxidation state. Bismuth ions can form complexes with anions, either to make bismuth salts, or to form complex anions which are then further complexed with one or more additional cation(s). Bismuth can also form covalent bonds to other atoms, such as sulfur.

As disclosed herein, a “bismuth-thiol compound” or “BT compound” is a compound that has a bismuth atom covalently bound to one, two or three other sulfur atoms present on one or more thiol compounds. The term “thiol” refers to a carbon-containing compound, or fragment thereof, containing an —SH group and can be represented by the general formula R-SH. These thiol compounds include compounds with one, two, three or more S atoms. Thiol compounds can have other functionality, such as alkyl, hydroxyl, carbocyclyl, heterocyclyl, aryl, heteroaryl, amino, and other substituents. Thiol compounds having two or more S atoms can chelate the bismuth atom, such that two S atoms from the same molecule covalently bond with the bismuth atom. Exemplary bismuth-thiol compounds are shown below:

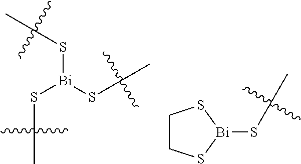

The term “thioalkyl”, as used herein, refers to an alkyl group substituted with a thiol group. A “thiol compound” as discussed above can include a thioalkyl as a substituent on the compound structure. A thiol compound can have, for example, one, two, three or more thioalkyl groups.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week.

“Coadministration” refers to the administration of the two agents in any manner in which the pharmacological effects of both agents are manifest in the patient at the same time. Thus, concomitant administration does not require that a single pharmaceutical composition, the same dosage form, or even the same route of administration be used for administration of both agents or that the two agents be administered at precisely the same time. However, in some situations, coadministration will be accomplished most conveniently by the same dosage form and the same route of administration, at substantially the same time.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “managing” includes therapeutic treatments as defined above. Managing includes achieving a steady state level of inflammation as determined by known methods in the art.

The term “lessen the severity” of inflammation or an inflammatory disease refers to an improvement in the clinical course of the inflammation on any measurable basis.

As used herein, the term “exacerbation” refers to an increase in the severity of symptoms during a course of a disease which is mostly associated with a worsening of quality of life. Exacerbations are quite frequent in patients with inflammatory diseases and conditions. By definition, exacerbations are simply a worsening and/or increase in symptoms.

An “effective amount”, as used herein, refers to an amount that is sufficient to achieve a desired biological effect. A “therapeutically effective amount”, as used herein refers to an amount that is sufficient to achieve a desired therapeutic effect. For example, a therapeutically effective amount can refer to an amount that is sufficient to improve at least one sign or symptom of an inflammatory disease or condition.

As used herein, the term “volumetric median diameter” or “VMD” of a particle in a composition or an aerosol is the particle size diameter identified such that half of the mass of the particles are contained in particles with larger diameter than the VMD, and half of the mass of the particles is contained in particles with smaller diameter than the VMD. VMD is typically measured by laser diffraction.

“Mass median aerodynamic diameter” or “MMAD” is a measure of the aerodynamic size of a dispersed aerosol particle. The aerodynamic diameter is used to describe an aerosolized particle in terms of its settling behavior, and is the diameter of a unit density sphere having the same settling velocity, generally in air, as the particle in question. The aerodynamic diameter encompasses particle shape, density, and physical size of a particle. As used herein, MMAD refers to the midpoint or median of the aerodynamic particle size distribution of an aerosolized particle determined by cascade impaction and/or laser time of flight and/or cascade impactor.

“Mass median diameter” or “MMD” is a measure of mean particle size. Any number of commonly employed techniques can be used for measuring mean particle size.

As used herein, “D90” refers to the 90% value of particle diameter (either the particle, such as a microparticle, or aerosolized particle). For example, if D90=1 μm, 90% of the particles are smaller than 1 μm. Similarly, “D80” refers to the 80% value of particle diameter (either the particle/microparticle or aerosolized particle), “D70” refers to the 70% value of particle diameter (either the particle/microparticle or aerosolized particle), “D60” refers to the 60% value of particle diameter (either the particle/microparticle or aerosolized particle), “D50” refers to the 500% value of particle diameter (either the particle/microparticle or aerosolized particle), “D40” refers to the 40% value of particle diameter (either the particle/microparticle or aerosolized particle), “D30” refers to the 30% value of particle diameter (either the particle/microparticle or aerosolized particle), “D20” refers to the 20% value of particle diameter (either the particle/microparticle or aerosolized particle), “D10” refers to the 10% value of particle diameter (either the particle/microparticle or aerosolized particle).

As used herein, “monodisperse” refers to a collection of particles (bulk or aerosol dispersion) comprising particles of a substantially uniform VMD or particle size diameter (PSD).

As used herein, the term “deposition efficiency” refers to the percentage of the delivered dose that is deposited into the area of interest. Thus, the deposition efficiency of a method and/or system for delivering an aerosolized medicament into the lungs is the amount by mass of the aerosol deposited into the lungs divided by the total amount of the aerosol delivered by the system to the nares.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of“substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

“Transmigration of leukocytes”, such as neutrophils, is the movement of leukocytes such as neutrophils from the blood of the patient to the site of inflammation, fibrosis, or infection. This can include, for example, the neutrophils crossing the endothelium and entering the inflamed tissue in a subject. This can include, for example, during tissue damage or related inflammation, the exit of leukocytes of blood vessels by adhering to and probing vascular endothelial cells (VECs), breaching endothelial cell-cell junctions, and transmigrating across the endothelium. The reduction of transmigration of leukocytes or neutrophils is thus the reduction or prevention of the movement of leukocytes such as neutrophils from the blood of the patient to the site of inflammation, fibrosis, or infection. Accordingly, treatment of “neutrophil-mediated inflammation/transmigration/infiltration” involves a therapeutic reduction in the migration of neutrophils into the target tissue. This may be particularly advantageous when treating a disease or condition characterized by neutrophil-dominant inflammation. Methods are accordingly provided herein for treating neutrophil-mediated or neutrophil-dominant inflammation or auto-inflammation. An undesirable side effect of neutrophil inhibition may be increased infection propensity. There is therefore a need for treatments as disclosed herein that inhibit neutrophil-mediated inflammation/transmigration/infiltration while simultaneously providing an anti-biotic effect.

### Methods of Use

The present disclosure provides methods of treatment, prevention, and/or relief of diseases caused by abnormal activation of an inflammasome or a cytokine in a subject in need thereof, comprising administering to the subject an effective amount of a BT composition comprising a BT compound, e.g., pravibismane.

In a specific embodiment, the disease may be an inflammation-related disease. The present disclosure therefore provides methods of treating an inflammation-related disease or condition in a subject in need thereof, comprising administering to the subject an effective amount of a BT composition comprising a BT compound, e.g., pravibismane.

In some embodiments, the disease caused by abnormal activation of anti-inflammatory cytokines (e.g., IL-10).

In some embodiments, inflammation-related disease or condition is regulated by or associated with the upregulated or down-regulated expression of a cytokine. In some embodiments, the cytokine is IL-10.

Accordingly, in some embodiments, the present disclosure provides methods of treating an inflammation-related disease or condition by increasing IL-10 production in a subject in need thereof, comprising administering to the subject an effective amount of a BT composition comprising a BT compound, e.g., pravibismane.

In some embodiments, the present disclosure provides methods of treating an inflammation-related or fibrosis-related disease or condition by modulating the production or expression of one or more cytokines in a subject in need thereof, comprising administering to the subject an effective amount of a BT composition comprising a BT compound, e.g., pravibismane.

In some embodiments, modulating the production or expression of one or more cytokines comprises increasing the production or expression or an anti-inflammatory cytokine and/or decreasing the production or expression of one or more pro-inflammatory cytokines. In some embodiments, modulating the production or expression of one or more cytokines comprises increasing the production or expression of one or more anti-inflammatory cytokine. In some embodiments, modulating the production or expression of a cytokine comprises decreasing the production or expression or one or more pro-inflammatory cytokines.

In some embodiments, the present disclosure also provides methods of treating an inflammation-related disease or condition by increasing production or expression of one or more anti-inflammatory cytokines in a subject in need thereof, comprising administering to the subject an effective amount of a BT composition comprising a BT compound, e.g., pravibismane.

In some embodiments, the one or more anti-inflammatory cytokines disclosed herein is IL-6 or IL-10. In some embodiments, the anti-inflammatory cytokine is IL-10.

In some embodiments, the one or more pro-inflammatory cytokines disclosed herein is IFN-γ, MIP-3α, MIP-1β, TNF-α, IL-22, IL-17A, IL-15, and IL-13. In some embodiments, the one or more pro-inflammatory cytokines disclosed herein is IFN-γ, MIP-3α, MIP-1β, TNF-α, and IL-22.

In some embodiments, the present methods of treating an inflammation-related disease or condition in a subject in need thereof provide a decrease in secretion of IFN-γ, MIP-3α, MIP-1β, TNF-α, IL-22, IL-17A, IL-15, and/or IL-13 compared to an untreated subject. In some embodiments, the present methods of treating an inflammation-related disease or condition in a subject in need thereof provide a decrease in secretion of IFN-γ, MIP-1, TNF-α, IL-17A, IL-15, and/or IL-13 compared to an untreated subject. In some embodiments, the present methods of treating an inflammation-related disease or condition in a subject in need thereof provide a decrease in secretion of MIP-3α, MIP-1β, and/or TNF-α compared to an untreated subject. In some embodiments, secretion of MIP-3α, MIP-1β, and/or TNF-α is decreased about 2- to 10-fold compared to an untreated subject. In some embodiments, secretion of MIP-3α, MIP-1β, and/or TNF-α is decreased about 3-fold compared to an untreated subject.

In some embodiments, the present methods of treating an inflammation-related disease or condition in a subject in need thereof increase secretion of IL-2 compared to an untreated subject. In some embodiments, IL-2 secretion is increased about 2- to 10-fold compared to an untreated subject. In some embodiments, IL-2 section is induced about 3-fold compared to an untreated subject.

In some embodiments, the present disclosure also provides methods of treating an inflammation-related disease or condition in a subject exhibiting a low level of IL-10 expression (compared to a subject not afflicted with an inflammatory condition), comprising administering to the subject an effective amount of a BT composition comprising a BT compound, e.g., pravibismane.

In some embodiments, the present disclosure further provides methods of treating an inflammation-related disease or condition characterized by depressed IL-10 expression, comprising administering to a subject in need thereof an effective amount of a BT composition comprising a BT compound, e.g., pravibismane.

In other embodiments, the present disclosure provides methods of treating an inflammation-related disease or condition, or a fibrosis-related disease or condition, or a combination thereof, responsive to a modulation of IL-10 or other anti-inflammatory cytokine expression, comprising administering to a subject diagnosed with an inflammatory condition, or a fibrotic condition, or combination thereof, an effective amount of a BT composition comprising a BT compound, e.g., pravibismane.

In another embodiment, the present disclosure provides methods for increasing IL-10 or other anti-inflammatory cytokine expression in a subject suffering from an inflammation-related disease or condition, a fibrosis-related condition, or combination thereof, comprising administering to the subject an effective amount of a BT composition comprising a BT compound, e.g., pravibismane.

In other embodiments, the present disclosure provides methods of treating an inflammation-related disease or condition, or a fibrosis-related disease or condition, or a combination thereof, responsive to a modulation of TNF-α or other inflammatory cytokine expression, comprising administering to a subject diagnosed with an inflammatory condition, or a fibrosis-related condition, or combination thereof, an effective amount of a BT composition comprising a BT compound, e.g., pravibismane.

In another embodiment, the present disclosure provides methods for decreasing TNF-α or other inflammatory cytokine expression in a subject suffering from an inflammation-related disease or condition, comprising administering to the subject an effective amount of a BT composition comprising a BT compound, e.g., pravibismane.

In other embodiments, the present disclosure provides methods of treating an inflammation-related disease or condition, or a fibrosis-related disease or condition, or a combination thereof, responsive to a modulation of cytokines such that the overall effect is to reduce inflammation and/or aberrant fibrosis, comprising administering to a subject diagnosed with an inflammatory condition, or a fibrotic condition, or combination thereof, an effective amount of a BT composition comprising a BT compound, e.g., pravibismane.

In some embodiments, the inflammation-related disease or condition, or the fibrosis-related condition, or combination thereof, is acute. In some embodiments, the inflammation-related disease or condition, or the fibrosis-related condition, or combination thereof, is chronic.

In some embodiments, the disease, or inflammatory and/or fibrotic disease or condition is an autoimmune disease, a pulmonary disease, a dermatologic disease, an infectious disease, a cardiovascular disease, a musculoskeletal disease, a gastrointestinal disease, a metabolic disease, a liver disease, a kidney disease, a pancreatic disease, a mucosal disease, a neoplastic disease, or a neurodegenerative disease.

In some embodiments, the autoimmune inflammatory and/or fibrotic disease is an inflammatory arthritide, such as arthritis (RA), ankylosing spondylitis (AS), or psoriatic arthritis. In some embodiments, the autoimmune inflammatory and/or fibrotic disease is autoimmune inflammation, Type 2 autoimmune pancreatitis, vasculitis, lupus erythematosus, Sjogren's syndrome, familial Mediterranean fever (FMF), cryopyrin-associated periodic syndromes (CAPS), or Still disease.

In some embodiments, the pulmonary inflammatory and/or fibrotic disease is cystic fibrosis (CF), chronic rhinosinusitis, nasal polyps, asthma, non-CF bronchiectasis, chronic obstructive pulmonary disease (COPD), idiopathic pulmonary fibrosis (IPF), sarcoidosis, systemic sclerosis associated interstitial lung disease, pulmonary fibrosis associated with rheumatoid arthritis, and VEXAS Syndrome.

In some embodiments, the epithelial inflammatory and/or fibrotic disease is atopic dermatitis, eczema, allergy, atopy, conjunctivitis, periodontitis, rhinitis, otitis media, laryngopharyngitis, tonsilitis, cellulitis, Behcet's disease, Sweet disease, lupus erythematosus, neutrophilic eccrine hidradenitis (NEH), Hidradenitis Suppurativa, psoriasis, generalized pustular psoriasis, pyoderma gangrenosum, subcorneal pustular dermatosis, rheumatoid neutrophilic dermatitis, Behçet's disease (BD), amicrobial pustulosis of the folds (APF), Crohn disease-associated neutrophilic urticarial dermatosis, system sclerosis (scleroderma), dermatofibrosis lenticularis disseminata, morphea, keloid formation, hypertrophic scars, eosinophilic fasciitis, stasis dermatitis, cutaneous tuberculosis, mycetoma, VEXAS Syndrome, and panniculitis.

In some embodiments, the gastrointestinal inflammatory and/or fibrotic disease is infectious colitis, Crohn's Disease, Bowel-Associated Dermatosis-Arthritis Syndrome (BADAS), ulcerative colitis, and inflammatory bowel disease (IBD).

In some embodiments, the cardiovascular inflammatory and/or fibrotic disease is cardiac fibrosis, myocarditis, atherosclerosis, and aortic aneurysm.

In some embodiments, the metabolic inflammatory and/or fibrotic disease is diabetes, e.g., type-2 diabetes.

In some embodiments, the liver inflammatory and/or fibrotic disease is a fatty liver disease, e.g., nonalcoholic fatty liver disease (NAFLD) or non-alcoholic steatohepatitis (NASH).

In some embodiments, the neurodegenerative inflammatory and/or fibrotic disease is Parkinson's disease, multiple sclerosis, fibromyalgia, neuro-neutrophilic disease, or Alzheimer's disease.

In some embodiments, the musculoskeletal inflammatory and/or fibrotic disease is gout and polychrondritis.

To further advance the therapeutic utility of the pharmaceutical compositions provided herein, the present disclosure describes the surprising and unexpected discovery that BT compositions comprising a BT compound (e.g., BisEDT) can influence the expression of inflammasomes, such as NLRP3 and AIM2, in infected and uninfected macrophage cells. Inflammasome expression is a characteristically rapid, innate immune response, which is often responsible for the downstream activation and expression of a broad range of cytokines. Accordingly, the BT compounds provided herein are believed to be an effective agent for use in the prevention and/or treatment of inflammation and associated diseases and conditions.

In some embodiments, the present disclosure provides methods of treating or reducing inflammation and methods of treating or reducing the effects of an inflammation-related disease or condition in a subject in need thereof, comprising administering an effective amount of a BT composition comprising a BT compound, e.g., pravibismane, to the subject.

In some embodiments, the inflammation or inflammation-related disease or condition is regulated by or associated with the activation of an inflammasome. In some embodiments, the inflammation or inflammation-related disease or condition is regulated by the activation of an inflammasome. In some embodiments, the inflammation or inflammation-related disease or condition is associated with the activation of an inflammasome. In some embodiments, the inflammasome is an NLR family inflammasome. In some embodiments, the inflammasome is the NLRP3 inflammasome or the AIM2 inflammasome. In some embodiments, the inflammasome is the NLRP3 inflammasome. In some embodiments, the inflammasome is the AIM2 inflammasome. Without being bound by any particular theory, the NLRP3 inflammasome is triggered by a pattern recognition receptor that can undergo activation in response to highly diverse extracellular stimuli, including molecules frequently found in pathogens (Pathogen-associated Molecular Patterns; PAMPs) and molecules released by damaged cells (Damage-associated Molecular Patterns; DAMPs).

In some embodiments, upon administration of the BT composition comprising a BT compound to the subject, the inflammasome activation is inhibited. In some embodiments, the inflammasome is inhibited in cells of the subject not having a microbial infection. In some embodiments, the inhibited inflammasome is an NLR family inflammasome. In some embodiments, the inhibited inflammasome is the NLRP3 inflammasome or the AIM2 inflammasome. In some embodiments, the inhibited inflammasome is the NLRP3 inflammasome. In a specific embodiment, the BT composition comprising a BT compound, e.g., pravibismane.

In some embodiments, upon administration of the BT composition comprising a BT compound to the subject, e.g. pravibismane, inflammasome gene expression is reduced. In some embodiments, inflammasome gene expression is reduced in cells of the subject not having a microbial infection. In some embodiments, administering a BT composition comprising a BT compound to the subject, e.g. pravibismane, decreases expression of NLRP3 and/or AIM2 inflammasomes.

In some embodiments, the administration of upon administration of the BT composition comprising a BT compound to the subject, e.g. pravibismane, additionally prevents, reduces, or treats a microbial infection, or prevents, reduces, or treats microbial products (e.g., biofilms and superantigens) in the subject. In some embodiments, the administration of the BT composition comprising a BT compound, e.g., pravibismane, additionally reduces or treats a microbial infection, or reduces or treats microbial products (e.g., biofilms and toxins such as superantigens) in the subject. By reducing or treating such microbial products, in some embodiments, the administration of the BT composition comprising a BT compound, e.g., pravibismane, additionally reduces the expression of inflammasomes.

In some embodiments, the microbial infection is at least partially located in, or in close apposition to, the inflammasome-expressing cells of the subject.

In some embodiments, the microbial infection is a bacterial, viral, and/or fungal infection. In some embodiments, the microbial infection is a bacterial infection. In some embodiments, the microbial infection is caused by bacteria. In some embodiments, the bacteria comprise an ESKAPE pathogen, which includes, Enterococcus faecium, Staphylococcus aureus, Klebsiella pneumoniae, Acinetobacter baumannii, Pseudomonas aeruginosa, and Enterobacter spp. In some embodiments, the microbial infection is caused by Gram-positive bacteria. In some embodiments, the microbial infection is caused by Gram-negative bacteria. In some embodiments, the microbial infection is caused by methicillin-resistant Staphylococcus aureus (MRSA, including community-associated [CA]-MRSA), methicillin-resistant Staphylococcus epidermidis (MRSE), and vancomycin-resistant Enterococcus (VRE). In some embodiments, the microbial infection is caused by Pseudomonas aeruginosa, Escherichia coli, Klebsiella pneumoniae, and Acinetobacter baumannii.

In some embodiments, upon administration of the BT composition comprising a BT compound, e.g., pravibismane, to a subject having a microbial infection, the inflammasome expression in infected cells is increased compared to inflammasome expression in infected cells not treated with the BT composition comprising a BT compound, e.g., pravibismane. In some embodiments, only cells of a subject having a microbial infection exhibit increased inflammasome expression. In some embodiments, release of the NLRP3 inflammasome and/or the AIM2 inflammasome is increased. In some embodiments, expression of the NLRP3 inflammasome is increased. In some embodiments, the inflammasome-expressing cells are macrophages, white blood cells, bronchial epithelial cells, alveolar epithelial cells, or combinations thereof. In some embodiments, the inflammasome-expressing cells are macrophages. In some embodiments, upon administration of the BT composition comprising a BT compound, e.g., pravibismane, to the subject, the inflammasome release from infected macrophages is increased compared to inflammasome release from infected macrophages not treated with the BT composition comprising a BT compound, e.g., pravibismane.

In some embodiments, upon administration of the BT composition comprising a BT compound, e.g., pravibismane, to a subject having a microbial infection, the infection is reduced. In some embodiments, upon administration of the BT composition comprising a BT compound, e.g., pravibismane, to a subject having a microbial infection, the infection in the inflammasome-expressing cells is reduced. In some embodiments, upon administration of the BT composition comprising a BT compound, e.g., pravibismane, to a subject having a microbial infection, the infection in the macrophages is reduced. In some embodiments, the macrophages are THP-1 macrophages. In some embodiments, the microbial infection is a bacterial infection described herein.

In some embodiments of the present methods, the inflammation or inflammation-related disease or condition is regulated by or associated with the activation of a cytokine, e.g., a proinflammatory cytokine. In some embodiments, the pro-inflammatory cytokine is selected from one of more of IL-1β, IL-6, IL-18, TNF-α, IL-17A, and INF-γ.

In some embodiments, the disease is caused by abnormal activation of a cytokine, such as IL-1β, IL-6, IL-17 (also known as IL-17A), IL-18, TNF-α, and INF-γ. In some embodiments, the disease caused by abnormal activation of a cytokine, such as IL-4, IL-6, IL-10, IL-11, and 11-13. In some embodiments of the present methods, administration of the BT composition comprising a BT compound disclosed herein, e.g., pravibismane, to a subject in need thereof, provides a reduction in the expression of one or more pro-inflammatory cytokines. In some embodiments, the one or more pro-inflammatory cytokines is IL-1p, IL-6, IL-17, IL-18, TNF-α, or INF-γ. In some embodiments, pro-inflammatory cytokine expression in the subject is decreased by about 5%, about 10%, about 15%, about 20%, about 25%, about 30%, about 35%, about 40%, about 45%, about 50%, about 55%, about 60%, about 65%, about 70%, or about 75% compared to a subject not treated with the BT composition comprising a BT compound, e.g., pravibismane.

In some embodiments of the present methods, administration of the BT composition comprising a BT compound disclosed herein, e.g., pravibismane, to a subject in need thereof provides an increase in the expression of one or more anti-inflammatory cytokines. In some embodiments, the one or more anti-inflammatory cytokines is IL-4, IL-6, IL-10, and IL-11. In some embodiments, anti-inflammatory cytokine expression in the subject is increased by about 5%, about 10%, about 15%, about 20%, about 25%, about 30%, about 35%, about 40%, about 45%, about 50%, about 55%, about 60%, about 65%, about 70%, or about 75% compared to a subject not treated with the BT composition comprising a BT compound, e.g., pravibismane.

In some embodiments, the present disclosure provides methods for modulating production of one or more anti-inflammatory cytokines disclosed herein (e.g., IL-10) in a subject suffering from an inflammation-related disease or condition, comprising administering to the subject an effective amount of a BT composition comprising a BT compound, e.g., pravibismane.

In another embodiment, the present disclosure provides methods for increasing IL-10 production in a subject suffering from an inflammation-related disease or condition, comprising administering to the subject an effective amount of an effective amount of a BT composition comprising a BT compound, e.g., pravibismane. In another embodiment, the IL-10 expression in the subject before treatment is not modulated or affected by a microbial source, such as a bacterial or viral source.

In some embodiments, the present disclosure provides methods for modulating production of one or more proinflammatory cytokines disclosed herein (e.g., TNF-α, IFN-γ, MIP-3α, MIP-1β, IL-22, IL-17A, IL-15, and/or IL-13) in a subject suffering from an inflammation-related disease or condition, comprising administering to the subject an effective amount of a BT composition comprising a BT compound, e.g., pravibismane.

In some embodiments, the present disclosure provides methods for decreasing TNF-α production in a subject suffering from an inflammation-related disease or condition, comprising administering to the subject an effective amount of a BT composition comprising a BT compound, e.g., pravibismane.

Also provided herein is a method of inhibiting inflammasome activity, comprising contacting a cell of a subject with an effective amount of the BT composition comprising a BT compound, e.g., pravibismane.

In some embodiments, the inhibited inflammasome is a NLRP3 or AIM2 inflammasome. In some embodiments, the inhibited inflammasome is a NLRP3 inflammasome.

In some embodiments, the subject does not have a microbial infection.

In some embodiments, the cells are human cells. In some embodiments, the cells are macrophages, white blood cells, bronchial epithelial cells, alveolar epithelial cells, or combinations thereof. In some embodiments, the cells are macrophages. In some embodiments, macrophages are THP-1 macrophages.

In some embodiments, contacting the cell with the BT composition comprising a BT compound, e.g., pravibismane, results in endocytosis of the BT compound such as pravibismane. In some embodiments, contacting the cell with the BT compound results in phagocytosis of the BT compound.

### BT Compounds and Particles Thereof

In some embodiments, the pharmaceutical composition is a bismuth-thiol (BT) composition that comprises a BT compound, e.g., pravibismane, suspended therein, wherein the BT composition comprises a plurality of particles/microparticles. In some embodiments, the BT composition comprises a plurality of particles/microparticles of a BT compound, e.g., pravibismane, suspended therein, wherein the particles/microparticles have a D90 of less than about 5 μm, less than about 4 μm, less than about 3 μm, less than about 2 μm, or less than about 1 μm. In some embodiments, the D90 of said particles/microparticles ranges from about 0.1 μm to about 3 μm, including any range or value therebetween. In some embodiments, the D90 of said particles/microparticles ranges from about 1 μm to about 3 μm. In some embodiments, the D90 of said particles/microparticles ranges from about 2 μm to about 3 μm. In some embodiments, the BT composition comprises a plurality of particles/microparticles of a BT compound, e.g., pravibismane, suspended therein, wherein the particles/microparticles have a D90 of less than about 2 μm. In some embodiments, the D90 of said particles/microparticles ranges from about 0.1 μm to about 1 μm. In some embodiments, the D90 of said particles/microparticles ranges from about 0.5 μm to about 2 μm. In some embodiments, the D50 of said particles/microparticles is less than or equal to about 0.6 μm to about 1.5 μm. In some embodiments, the D50 of said particles/microparticles is less than or equal to about 0.6 μm to about 1.3 μm. In some embodiments, the D50 of said particles/microparticles is less than or equal to about 1.5 μm, about 1.4 μm, about 1.3 μm, about 1.2 μm, about 1.1 μm, about 1 μm, about 0.9 μm, about 0.8 μm, about 0.7 μm, about 0.6 μm, about 0.5 μm, about 0.4 μm, about 0.3 μm, or about 0.2 μm, including all ranges and values therebetween. In some embodiments, the D10 of said particles/microparticles is less than or equal to about 0.2 μm to about 0.9 μm. In some embodiments, the D10 of said particles/microparticles is less than or equal to about 0.3 μm to about 0.7 μm. In some embodiments, the D10 of said particles/microparticles is less than or equal to about 0.9 μm, about 0.8 μm, about 0.7 μm, about 0.6 μm, about 0.5 μm, about 0.4 μm, about 0.35 μm, about 0.3 μm, about 0.25 μm, or about 0.2 μm, including all ranges and values therebetween. In some embodiments, the D50 of said particles/microparticles is less than or equal to about 1.0 μm.

In a specific embodiment, the D90 of said particles/microparticles is less than or equal to: 4.5 μm, or 4.0 μm, or 3.5 μm, or 3.0 μm, or 2.5 μm, or 2.0 μm, or 1.9 μm, or 1.8 μm, or μm 1.7 μm, or 1.6 μm, or 1.5 μm or any ranges therebetween. In a specific embodiment, the D90 of said particles/microparticles is less than or equal to 2.0 μm. In another specific embodiment, the D90 of said particles/microparticles is less than or equal to 1.9 μm. In another specific embodiment, the D90 of said particles/microparticles is less than or equal to 1.6 μm. In another specific embodiment, the D90 of said particles/microparticles is less than or equal to 1.0 μm. In another specific embodiment, the D50 of said particles/microparticles is less than or equal to 2.5 μm, or 2.0 μm, or 1.5 μm, or 1.3 μm, or 1.2 μm, or 1.1 μm, or 1.0 μm, or 0.9 μm, or 0.87 μm, or 0.72 μm or any ranges therebetween. In another specific embodiment, the D10 of said particles/microparticles is less than or equal to 0.9 μm, or 0.8 μm, or 0.7 μm, or 0.6 μm, or 0.50 μm, or 0.40 μm, or 0.39 μm, or 0.38 μm, or 0.37 μm, or 0.36 μm, or 0.35 μm, or 0.34 μm, or 0.33 μm, or any ranges therebetween.

In some embodiments, the particles/microparticles have a D90 of less than about 2 μm, a D50 of less than or equal to about 0.6 μm to about 1.5 μm, and/or a D10 of less than or equal to about 0.2 μm to about 0.9 μm. In some embodiments, the particles/microparticles have a D90 of less than about 2 μm, a D50 of less than or equal to about 0.7 μm to about 1.3 μm, and/or a D10 of less than or equal to about 0.3 μm to about 0.7 μm.

In some embodiments, the bismuth-thiol composition comprises a plurality of particles/microparticles of a bismuth-thiol (BT) compound, e.g., pravibismane, suspended therein, wherein substantially all of said particles/microparticles having a volumetric mean diameter of from about 0.4 μm to about 5 μm. In some embodiments, at least 60%, 65%, 70%, 75%, 80%, 90%, or 95% of the particles/microparticles have a volumetric mean diameter of from about 0.4 μm to about 5 μm. In some embodiments, at least 60%, 65%, 70%, 75%, 80%, 90%, or 95% of the particles/microparticles have a volumetric mean diameter of from about 0.4 μm to about 3 μm, or from about 0.3 μm to about 2 μm, or from about 0.4 μm to about 2 μm, or from about 0.8 μm to about 1.8 μm, or from about 0.8 μm to about 1.6 μm, or from about 0.3 μm to about 0.9 μm, or from about 1.0 μm to about 2.0 μm, or from about 1.0 μm to about 1.8 μm, or any narrow ranges between the specific ranges described above.

In some embodiments, at least 60%, 65%, 70%, 75%, 80%, 90%, or 95% of the particles/microparticles have a volumetric mean diameter of from about 0.5 μm to about 3 μm. In some embodiments, substantially all of the particles/microparticles have a VMD of from about 0.5 μm to about 3 μm.

In some embodiments, at least 60%, 65%, 70%, 75%, 80%, 90%, or 95% of the particles/microparticles have a volumetric mean diameter of from about 0.6 μm to about 2.5 μm. In some embodiments, substantially all of the particles/microparticles have a VMD of from about 0.6 μm to about 2.5 μm.

In some embodiments, at least 60%, 65%, 70%, 75%, 80%, 90%, or 95% of the particles/microparticles have a volumetric mean diameter of from about 1 μm to about 2 μm. In some embodiments, substantially all of the particles/microparticles have a VMD of from about 1 μm to about 2 μm.

In some embodiments, the BT composition comprises a BT compound selected from:

bismuth-2,3-dimercaptopropanol (2:3 molar ratio, BisBAL)
bismuth-dithioerythritol (2:3 molar ratio, BisERY)
bismuth-4-methyl-1,2-benzenedithiol (2:3 molar ratio, BisTOL)
bismuth-2,3-butanedithiol (BisBDT)
bismuth-2,3-butanedithiol, 2-mercaptopyridine N-oxide (2:1:2 molar ratio, BisBDT/PYR)
bismuth-2,3-dimercaptopropanol, 2-mercaptopyridine N-oxide (2:1:2 molar ratio,
BisBAL/PYR)
bismuth-1,2-ethanedithiol, 2-mercaptopyridine N-oxide (2:1:2 molar ratio, BisEDT/PYR)
bismuth-4-methyl-1,2-benzenedithiol, 2-mercaptopyridine N-oxide (2:1:2 molar ratio,
BisTOL/PYR)
bismuth-1,3-propanedithiol, 2-mercaptopyridine N-oxide (2:1:2 molar ratio, BisPDT/PYR)
bismuth-dithioerythritol, 2-mercaptopyridine N-oxide (2:1:2 molar ratio, BisERY/PYR)
bismuth-1-mercapto-2-propanol, 1,2-ethanedithiol (1:1:1 molar ratio, BisHPT/EDT)
bismuth with ethanedithiol and 2-mercaptobenzoimidazole (BisEDT/2MBI (1:1))
bismuth with ethanedithiol and 2-mercaptopyrimidine (BisEDT/SPN (2MPMD) (1:1))
bismuth with ethanedithiol and 3-mercapto-1,2,4-triazole (BisEDT/3MTZ (1:1))
bismuth with ethanedithiol and 1-propane thiol (BisEDT/PT (1:1))
bismuth with ethanedithiol and cysteamine (BisEDT/CSTMN (1:1))
bismuth with ethanedithiol and 3-mercaptopropionic acid (BisEDT/3MPA (1:1))
bismuth with lipoic acid (reduced) (BisALA (BisLipo) (1:1.5))
bismuth with 2-mercaptolpyridine N-oxide and 2-mercaptobenzoimidazole (BisPYR/2MBI
(1:1))
bismuth with 2-mercaptolpyridine N-oxide and cysteamine (BisPYR/CSTMN (1:1))
bismuth with 2,3-dimercapto-1-propanol and 2-mercaptobenzoimidazole (BisBAL/2MBI
(1:1))
bismuth with 2,3-dimercapto-1-propanol and cysteamine (BisBAL/CSTMN (1:1))
bismuth with 3,4 dimercapto toluene and 2-mercaptobenzoimidazole (BisTOL/2MBI (1:1))
bismuth with 3,4 dimercapto toluene and cysteamine (BisTOL/CSTMN (1:1))
bismuth with 2-mercapto pyridine (BisEDT/MPYR)

In some embodiments, the BT composition comprises a compound selected from Bis-BAL, BisEDT, Bis-dimercaprol, Bis-DTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, Bi-sPyr/EDT, Bis-Pyr/PDT, Bis-Pyr/Tol, Bis-Pyr/Ery, bismuth-1-mercapto-2-propanol, and BisEDT/2-hydroxy-1-propanethiol. In other embodiments, the BT compound is selected from one or more of BisEDT, Bis-Bal, Bis-Pyr, Bis-Ery, Bis-Tol, Bis-BDT, or BisEDT/2-hydroxy-1-propane thiol. As used herein, MB-1B3 (or MB-1-B3) refers to BisEDT; MB-6 refers to BisBDT; MB-8-2 refers to BisBDT/PYR; and MB-11 refers to BisEDT/PYR In some embodiments, the BT composition comprises Bis-Bal or BisEDT. In some embodiments, the BT compositions comprise BisEDT (pravibismane).

In some embodiments, pravibismane has the following structure:

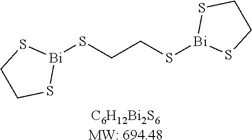

### Bismuth-Thiol (BI) Compositions of the Disclosure

The compositions of the present disclosure comprising a BT compound, e.g., pravibismane, can be utilized to treat a subject in need thereof. In some embodiments, the subject is a mammal such as a human, or a non-human mammal. When administered to the subject, such as a human, the composition or the compound is preferably administered as a pharmaceutical composition comprising, for example, a BT compound of the disclosure and a pharmaceutically acceptable carrier. In specific embodiments, the BT compound is pravibismane.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols and sugar alcohols, such as glycerin, sorbitol, mannitol, xylitol, erythritol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances, including salts such as sodium chloride, employed in pharmaceutical formulations.

In some embodiments, the pharmaceutically acceptable carriers for use in the present compositions include aqueous solutions such as water, physiologically buffered saline, physiologically buffered phosphate, or other solvents or vehicles such as glycols, glycerol, oils such as olive oil, or injectable organic esters. In some embodiments, when such pharmaceutical compositions are for human administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as lyophile for reconstitution, powder, solution, syrup, injection or the like. The composition can also be present in a solution suitable for topical administration. In a specific embodiment, the topical administration is for treatment of epithelial surfaces.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose, or dextrans; antioxidants, such as ascorbic acid or glutathione; chelating agents; low molecular weight proteins; salts; or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer. In some embodiments, the pharmaceutical compositions of the present disclosure are liposome-free.

In some embodiments, the BT composition further comprises one or more carriers selected from animal and vegetable fats, oils, waxes, paraffins, starch, tragacanth, xanthan gum, cellulose derivatives, polyethylene glycols, silicones, bentonites, silicic acid, polymers, talc, and zinc oxide. In some embodiments, the carrier is methylcellulose. In some embodiments, the carrier is poly(methyl methacrylate).

The BT composition can comprise any suitable concentration of a BT compound disclosed herein. In some embodiments, the BT composition is administered as a dosage comprising from about 0.25 mg/mL to about 15 mg/mL, from about 0.4 mg/mL to about 15 mg/mL, from about 0.6 mg/mL to about 15 mg/mL, from about 0.6 mg/mL to about 100 mg/mL, from about 5 mg/mL to about 100 mg/mL, from about 10 mg/mL to about 100 mg/mL, from about 25 mg/mL to about 100 mg/mL, from about 50 mg/mL to about 100 mg/mL, from about 0.8 mg/mL to about 15 mg/mL, from about 1 mg/mL to about 10 mg/mL, from 2.5 mg/mL to about 10 mg/mL, from about 4 mg/mL to about 10 mg/mL, from about 5 mg/mL to about 10 mg/mL, from about 6 mg/mL to about 10 mg/mL, 0.6 mg/mL to about 6 mg/mL, from about 4 mg/mL to about 15 mg/mL, from about 6 mg/mL to about 15 mg/mL, from about 50 μg/mL to about 750 μg/mL, from about 75 μg/mL to about 500 μg/mL, from about 100 μg/mL to about 250 μg/mL, from about 100 μg/mL to about 150 μg/mL, or from about 75 μg/mL to about 150 μg/mL of the BT compound, e.g. pravibismane.

In some embodiments, the BT composition is a suspension disclosed herein formulated in polysorbate (e.g., polysorbate 80) and/or in a buffer. For example, in some embodiments, the BT composition is a suspension formulated in about 0.1% polysorbate 80 to about 1.0% polysorbate 80, including all ranges therebetween. For example, the BT composition is a suspension formulated in about 0.1% polysorbate 80, about 0.2% polysorbate 80, about 0.3% polysorbate 80, about 0.4% polysorbate 80, about 0.5% polysorbate 80, about 0.6% polysorbate 80, about 0.7% polysorbate 80, about 0.8% polysorbate 80, about 0.9% polysorbate 80, or about 1% polysorbate 80. In some embodiments, the BT composition is a suspension of a BT compound, e.g., pravibismane, formulated in about 0.5% polysorbate 80.

A variety of buffers may be used in the context of the present disclosure and will be readily apparent to a person having ordinary skill in the art. For example, in some embodiments, suitable buffers include sodium or potassium citrate, citric acid, phosphate buffers such as sodium phosphate, boric acid, sodium bicarbonate and various mixed phosphate buffers including combinations of Na₂HPO₄, NaH₂PO₄ and KH₂PO₄. In some embodiments, a sodium phosphate buffer is used. In some embodiments, sodium citrate buffer is used.

The formulation pH may vary from about 5 to about 10. In some embodiments, the formulation pH is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10. In some embodiments, the formulation pH is about 7.4.

In a specific embodiment, the BT composition comprises a BT compound suspended therein, wherein the BT composition comprises a plurality of particles/microparticles, wherein the D90 of said particles/microparticles is less than or equal to about 1.6 m. In a specific embodiment, the BT composition comprises pravibismane or other BT compound at a concentration greater than about 0.1 mg/mL, about 0.05% to about 1.0% polysorbate 80, optionally about 0.05 to 40 mM sodium chloride, and optionally about 2 to 20 mM sodium phosphate at about pH. 7.4.

In a specific embodiment, the BT composition comprises pravibismane at a concentration greater than about 0.1 mg/mL, about 0.05% to about 1.0% polysorbate 80, about 0.05 to 40 mM sodium chloride, and optionally about 2 to 20 mM sodium phosphate at about pH. 7.4. In some embodiments, the BT composition is a suspension of pravibismane in about 0.5% polysorbate 80 in sodium phosphate buffer at a pH of about 7.4. In some embodiments, pravibismane is present in the composition at a concentration ranging from about 100 μg/mL to about 1000 mg/mL including all integers and ranges therebetween. For example, in some embodiments, pravibismane is present in the composition at a concentration ranging from about 100 μg/mL, 200 μg/mL, 300 μg/mL, 400 μg/mL, 500 μg/mL, 600 μg/mL, 700 μg/mL, 800 μg/mL, 900 μg/mL, 1000 μg/mL, 10 mg/mL, 25 mg/mL, 50 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, 525 mg/mL, 550 mg/mL, 575 mg/mL, 600 mg/mL, 625 mg/mL, 650 mg/mL, 675 mg/mL, 700 mg/mL, 725 mg/mL, 750 mg/mL, 775 mg/mL, 800 mg/mL, 825 mg/mL, 850 mg/mL, 875 mg/mL, 900 mg/mL, 925 mg/mL, 950 mg/mL, 975 mg/mL, to about 1000 mg/mL. In some embodiments, pravibismane is present in the composition at a concentration ranging from about 100 μg/mL to about 1000 μg/mL.

In some embodiments, the composition osmolality may need to be further adjusted with an additive such as NaCl or TDAPS to achieve a desired osmolality. For example, in some embodiments, the osmolality of the composition is adjusted with sodium chloride to an osmolality ranging from about 100 mOsmol/kg to about 500 mOsmol/kg, including all integers and ranges therebetween. In some embodiments, the osmolality of the composition is from about 290 mOsmol/kg to about 310 mOsmol/kg. For example, in some embodiments, the osmolality of the composition is about 290 mOsmol/kg, 291 mOsmol/kg, 292 mOsmol/kg, 293 mOsmol/kg, 294 mOsmol/kg, 295 mOsmol/kg, 296 mOsmol/kg, 297 mOsmol/kg, 298 mOsmol/kg, 299 mOsmol/kg, 300 mOsmol/kg, 301 mOsmol/kg, 302 mOsmol/kg, 303 mOsmol/kg, 304 mOsmol/kg, 305 mOsmol/kg, 306 mOsmol/kg, 307 mOsmol/kg, 308 mOsmol/kg, 309 mOsmol/kg, to about 310 mOsmol/kg. In some embodiments, the osmolality is about 300 mOsmol/kg.

In some embodiments, the BT composition is a suspension of a BT compound, e.g., pravibismane, in polysorbate (e.g., polysorbate 80) in a buffer (e.g., sodium phosphate buffer). In some embodiments, the BT composition is a suspension of the BT compound in about 0.5% polysorbate 80 in a sodium phosphate buffer at a pH of about 7.4. In some embodiments, the BT composition is a suspension of pravibismane in about 0.5% polysorbate 80 in a sodium phosphate buffer at a pH of about 7.4, wherein the composition has an osmolality of about 300 mOsmol/kg (e.g., adjusted to 300 mOsmol/kg with sodium chloride). In some embodiments, the BT compound is pravibismane, and the pravibismane is present at a concentration of about 100 μg/mL, 250 μg/mL, 500 μg/mL, 750 μg/mL, 1000 μg/mL, 2.5 mg/mL, 10 mg/mL, 25 mg/mL, 50 mg/mL, 75 mg/mL, or about 100 mg/mL. In some embodiments, the BT composition is a suspension of pravibismane with or without a surfactant (e.g., polysorbate 80) and with or without a buffer (e.g., sodium phosphate buffer).

### Formulations and Administration Thereof

The formulations of the present disclosure can conveniently be presented in unit dosage form and can be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect.

In some embodiments, the BT composition comprising a BT compound, such as pravibismane, is a powder, spray, ointment, paste, cream, lotion, solution, patch, suspension or gel. In some embodiments, the BT composition is a solution. Liquid dosage forms include pharmaceutically acceptable emulsions, lyophiles for reconstitution, microemulsions, solutions, suspensions, gels, syrups and elixirs. In addition to the active ingredient, the liquid dosage forms can contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (such as cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the topical compositions can also include adjuvants such as wetting agents, emulsifying and suspending agents, and preservative agents.

Powders and sprays can contain, in addition to an active compound, excipients such as methylcellulose, sodium chloride, PMMA, lactose, talc, silicic acid, aluminum hydroxide, calcium silicates and polyamide powder, dipalmitoylphosphatidylcholine (DPPC), leucine, polyethylene glycol, or mixtures of these substances. Sprays can additionally contain customary propellants, such as chlorofluorohydrocarbons and volatile unsubstituted hydrocarbons, such as butane and propane.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, the pharmaceutical compositions disclosed herein are formulated to be administered orally, nasally, transdermally, topically, pulmonarily, inhalationally, buccally, sublingually, intraperitoneally, subcutaneously, intramuscularly, intravenously, intrapleurally, intrathecally, intraportally, parenterally, or as an implant. In some embodiments, the pharmaceutical compositions disclosed herein are formulated to be administered topically or transdermally.

Dosage forms for the topical or transdermal administration include powders, sprays, ointments, pastes, creams, lotions, gels, solutions, patches and inhalants. The active compound can be mixed under sterile conditions with a pharmaceutically acceptable carrier, and with any preservatives or buffers that can be required.

The ointments, pastes, creams and gels can contain, in addition to an active compound, one or more excipients or carriers, such as animal and vegetable fats, oils, waxes, paraffins, starch, tragacanth, cellulose derivatives, polyethylene glycols, silicones, bentonites, silicic acid, talc, polymers, salts, and zinc oxide, or mixtures thereof. In some embodiments, the BT composition is in the form of an aqueous solution. In some embodiments, the excipient comprises a salt selected from sodium chloride or potassium chloride. In some embodiments, the excipient comprises sodium chloride.

In some embodiments, the BT composition is a suspension formulation which is intended for pulmonary delivery, e.g., to treat an inflammatory disease of the lung. For example, the BT composition is a suspension formulation that is ultimately administered by inhalation either orally and/or nasally. Accordingly, in some embodiments, the BT composition of the present disclosure is an aerosol that can be delivered by a device such as a nebulizer.

In some embodiments, the BT composition is administered by inhalation, orally or nasally, using an aerosol device, such as a nebulizer. A nebulizer can administer the BT composition topically to the lung tissue, which can include the lung mucosa, the alveoli (e.g., deep lung alveoli), the bronchi and/or the bronchioles. Thus, in some embodiments, the present disclosure provides for administration of the BT composition to the deep lung region of the lung (e.g., the deep lung alveoli). Local topical administration of the BT composition provides several key advantages over systemic antibiotic therapies. The term “systemic” refers to administration of a medication into the circulatory system of the subject such that the majority of the entire body can be exposed. Systemic administration of a medication can occurs enterally (absorption through the gastrointestinal tract, e.g., oral administration) or parenterally (absorption through injection or infusion, e.g., intravenously).

Known nebulizers, such as PARI LC PLUS®, can administer the disclosed compositions as an aqueous solution, optionally in buffered saline. The solution can be provided to the subject in the form of an ampule for use in the nebulizer. The nebulizer can be reusable and includes a compressor that provides the formulation over a period of time, such as about 10-15 minutes or longer. Known compressors, such as APRI Vios Air and DeVilbiss Pulmo-aide, are suitable for administration. The nebulizer administers the formulation topically to the lung tissues, such as mucosa, the bronchi and/or the bronchioles, alveoli, deep lung alveoli. In other embodiments, a nose-only aerosol device can be used for administration of the formulation.

An exemplary BT composition formulation is a neutral pH, isotonic, buffered aqueous solution of BT compound particles/microparticles with a nonionic surfactant. In certain embodiments, the buffer is a phosphate buffer with added NaCl. In some embodiments, the suspension particle size is a D₅₀ of about 0.2 to 2 μm. The formulation can be delivered using commercially available compressed air jet nebulizer. In some embodiments, the formulation concentration is about 0.1 μg/mL to about 100 mg/mL.

In some embodiments, the present disclosure provides an aerosol comprising a plurality of dispersed liquid droplets in a gas, said liquid droplets comprising a BT composition comprising a BT compound, e.g., pravibismane, suspended therein.

In some embodiments, the liquid droplet comprises particles/microparticles of a BT compound selected from BisBAL, BisEDT, Bis-dimercaprol, BisDTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, BisPyr/EDT, Bis-Pyr/PDT, Bis-Pyr/Tol, Bis-Pyr/Ery, bismuth-1-mercapto-2-propanol, BisEDT/CSTMN (1:1), BisPYR/CSTMN (1:1), BisBAL/CSTMN (1:1), BisTOL/CSTMN (1:1), and BisEDT/2-hydroxy-1-propanethiol. In some embodiments, the BT compound is selected from BisEDT, Bis-Bal, Bis-Pyr, Bis-Ery, Bis-Tol, Bis-BDT, or BisEDT/2-hydroxy-1-propane thiol. In some embodiments, the BT compound is BisEDT or BisBAL. In some embodiments, the BT compound is BisEDT.

In some embodiments, the liquid droplets comprise a bismuth-thiol (BT) composition that comprises particles/microparticles of a BT compound (e.g., pravibismane) suspended therein, the BT compound particles/microparticles having a D90 of less than about 10 μm. In some embodiments, the particles/microparticles have a D90 of less than about 5 μm, less than about 4 μm, less than about 3 μm, less than about 2 μm, or less than about 1 μm. In some embodiments, the particles/microparticles have a D90 of less than about 2 μm. In some embodiments, the particles/microparticles have a D90 ranging from about 0.1 μm to about 5 μm, or about 0.1 μm to about 4 μm, or about 0.1 μm to about 3 μm, or about 0.1 μm to about 2 μm, including all ranges and values therebetween. In some embodiments, the particles/microparticles have a D90 ranging from about 1 μm to about 5 m, or about 1 μm to about 4 μm, or about 1 μm to about 3 μm, or about 0.5 μm to about 1 μm.

In some embodiments, the liquid droplets comprise a bismuth-thiol (BT) composition that comprises particles/microparticles of a BT compound (e.g., pravibismane) suspended therein, the BT compound particles/microparticles having a volumetric mean diameter (VMD) from about 0.4 μm to about 5 μm. In some embodiments, at least 60%, 65%, 70%, 75%, 80%, 90%, or 95% of the particles/microparticles have a VMD of from about 0.4 μm to about 5 μm, or from about 0.6 μm to about 2.5 μm, or from about 0.7 μm to about 4 μm, or from about 0.7 μm to about 3.5 μm, or from about 0.7 μm to about 3.0 μm, or from about 0.9 μm to about 3.5 μm, or from about 0.9 μm to about 3 μm, or from about 0.8 μm to about 1.8 μm, or from about 0.8 μm to about 1.6 μm, or from about 0.4 μm to about 0.9 μm, or from about 1.0 μm to about 2.0 μm, or from about 1.0 μm to about 1.8 μm and all ranges therebetween. In some embodiments, at least 60%, 65%, 70%, 75%, 80%, 90%, or 95% of the particles/microparticles have a VMD of from about 0.6 μm to about 2.5 μm, or from about 0.8 μm to about 1.6 μm, or from about 0.9 μm to about 3.5 μm, or from about 0.9 μm to about 3 μm, or from about 0.9 μm to about 1.4 μm, or from about 1.0 μm to about 2.0 μm, or from about 1.0 μm to about 1.8 μm and all ranges therebetween.

In some embodiments, at 600%, 65%, 700, 75%, 800%, 90%, or 95% of the liquid droplets have a mass median aerodynamic diameter (MMAD) from about 0.4 μm to about 5 μm when measured by laser time of flight and/or cascade impactor. In some embodiments, at least 60%, 65%, 70%, 75%, 80%, 90%, or 95% of the liquid droplets have a MMAD of from about 0.4 μm to about 7 μm, or from about 0.5 μm to about 5 μm, or from about 0.7 μm to about 4 μm, or from about 0.7 μm to about 3.5 μm, or from about 0.8 μm to about 3.5 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, including all ranges therebetween. In some embodiments, at least 60%, 65%, 70%, 75%, 80%, 90°/c, or 95% of the liquid droplets have a MMAD of from about 0.8 μm to about 1.6 μm, or from about 0.9 μm to about 3.5 μm, or from about 0.9 μm to about 5 μm, or from about 0.9 μm to about 2.4 μm, or from about 1.0 μm to about 2.0 μm, or from about 1.0 μm to about 1.8 μm, and all ranges therebetween.

The aerosols of the present disclosure can have a very narrow MMAD distribution, as defined herein, which is beneficial because of the need to concentrate the particle mass in the target size range and minimize or eliminate the fraction of the product that is outside of the respirable range or ‘fines’, i.e., particles of typically less than 0.4 sun diameter. The ability to create a narrow droplet size distribution in the appropriate size range provides control of the initial evaporation rate and allows for high deposition efficiency. The limiting factor in terms of the lower limit of particle aerosol droplet size is the BT particle size (e.g., the pravibismane particle size). An aerosolized droplet cannot be smaller than the pravibismane particulate size. As such, the BT particle/microparticle size distribution, as well as the uniformity and consistent reproducibility of the BT particulate/microparticulate size distribution, are important beneficial characteristics to support the generation of a safe, effective, and efficient aerosolized pravibismane drug product for inhalation purposes. Accordingly, in some embodiments, the aerosols of the present disclosure effectuate a deposition efficiency of greater than 3%, greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, and greater than 80%. In some embodiments, the deposition efficiency refers to deposition to the deep lung region of lung, for example, to the deep lung alveoli. In some embodiments, the aerosols of the present disclosure effectuate a deposition efficiency upon aerosolization via a nebulizer. In some embodiments, the plurality of liquid droplets is dispersed in a continuous gas phase.

In some embodiments, the plurality of liquid droplets is dispersed in a continuous gas phase.

In some embodiments, the droplets further comprise polysorbate 80 (e.g., from about 0.05% to about 1%) and optionally a buffer (e.g., sodium phosphate or sodium citrate) at a pH of about 7.4; and/or sodium chloride.

In some embodiments, the BT compound (e.g., pravibismane) is suspended in the liquid droplet. The BT compounds of the present disclosure have little to no solubility in conventional solvents and aerosol carriers and therefore exist substantially as a suspension of BT particles in the aerosol droplet. For example, in some embodiments, the BT compound (such as pravibismane) is less than 1% soluble in the aerosol carrier and therefore exists primarily (>99%) as a solid.

In some embodiments, the aerosol comprises the BT compound in any concentration disclosed herein. In some embodiments, the BT compound is at a concentration greater than about 0.1 mg/mL, about 0.05% to about 1.0% polysorbate 80, about 0.05 to 40 mM sodium chloride, and optionally about 2 to 20 mM sodium phosphate at about pH. 7.4. For example, in some embodiments, the BT composition comprises pravibismane at a concentration greater than about 0.25 mg/mL, about 0.5% polysorbate 80, about 10 mM sodium chloride, and about 10 mM sodium phosphate at about pH 7.4.

In another embodiment, after delivering the aerosolized composition to a subject, at least 60%, 65%, 70%, 75%, 80%, 90%, or 95% of the dose is deposited on the lung to treat an inflammation disease of the lung, as opposed to the oropharyngeal region and the conducting airways. In a specific embodiment, at least 80% of the dose is deposited on the lung, as opposed to the oropharyngeal region and the conducting airways. In another specific embodiment, at least 90% of the dose is deposited on the lung, as opposed to the oropharyngeal region and the conducting airways. In another specific embodiment, the percent deposition is determined using a PARI LC PLUS® jet nebulizer to administer to the rats with the formulations described herein.

In some embodiments, when the aerosol is deposited to the lung (e.g., to the deep lung alveoli), the BT compounds have an average half-life of at least 2 days. For example, the BT compounds have an average half-life of about 2, 3, 4, or 5 days. In some embodiments, the BT compound is pravibismane. In a specific embodiment, the lung tissue half-life of pravibismane is 30 hrs or more, 40 hrs or more, 50 hrs or more, 60 hrs or more, 70, hrs or more, 80 hrs or more, 90 hrs or more, 100 hrs or more, 110 hrs or more, 125 hrs or more, or 150 hrs or more. In a specific embodiment, the lung tissue half-life is after a single dose via inhalation.

Compositions disclosed herein can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, by ionizing radiation (gamma photons for example), autoclaving, or by incorporating sterilizing agents in the form of sterile solid compositions that can be dissolved in sterile water, or some other sterile injectable medium immediately before use.

### Administration

In some embodiments, the BT composition of the present disclosure is administered three times per day, two times per day, once daily, every other day, once every three days, once every week, once every other week, once monthly, to once every other month. In certain embodiments, the BT composition is administered once daily. In certain embodiments, the BT composition is administered once weekly. In certain embodiments, the BT composition is administered once every other week. In some embodiments, the BT composition is administered chronically in a 4 week on/4 week off dosing schedule. In some embodiments, the BT composition is administered chronically, for example as part of a background therapy. As will be appreciated by a person having ordinary skill in the art, the administration frequency may depend on a number of factors including dose and administration route. For example, if a BT composition comprising pravibismane is administered via an aerosol administration, a low dose such as 100-1000 μg/mL may be administered once or twice daily; however, a high dose such as 2.5-10 mg/mL may be administered e.g., once or twice a week or less frequently.

### NUMBERED EMBODIMENTS

1. A method of treating an inflammation-related disease or condition in a subject in need thereof, comprising administering to the subject an effective amount of a bismuth-thiol (BT) composition that comprises a BT compound.
2. The method of embodiment 1, wherein the inflammation-related disease or condition is an autoimmune disease, a cardiovascular disease, a gastrointestinal disorder, a metabolic disease, a liver disease, a neurodegenerative disease, an inflammatory disease of the lung, or an inflammatory condition of the skin.
3. The method of embodiment 1 or 2, wherein the autoimmune disease is rheumatoid arthritis (RA), ankylosing spondylitis (AS), or psoriatic arthritis, the cardiovascular disease is heart disease, the inflammatory disease of the lung is chronic obstructive pulmonary disease (COPD) or asthma, the gastrointestinal disorder is ulcerative colitis or Crohn's disease, the metabolic disease is type-2 diabetes, the liver disease is nonalcoholic fatty liver disease (NAFLD) or non-alcoholic steatohepatitis (NASH), the neurodegenerative disease is Parkinson's disease or Alzheimer's disease, the inflammatory disease of the skin is acne vulgaris, atopic dermatitis, or psoriasis.
4. The method of any one of embodiments 1-3, wherein the inflammation-related disease or condition is an inflammatory disease or condition of the skin.
5. The method of embodiment 4, wherein the inflammatory disease or condition of the skin is psoriasis or acne vulgaris.
6. The method of any one of embodiments 1-5, wherein the inflammatory disease or condition of the skin is psoriasis.
7. A method of treating an inflammation-related disease or condition by increasing IL-10 production in a subject in need thereof, comprising administering to the subject an effective amount of a bismuth-thiol (BT) composition.
8. A method of treating an inflammation-related disease or condition in a subject exhibiting a low level of IL-10 expression relative to a subject not afflicted with an inflammatory condition, comprising administering to the subject an effective amount of a bismuth-thiol (BT) composition.
9. A method of treating an inflammation-related disease or condition characterized by depressed IL-10 expression, comprising administering to a subject in need thereof an effective amount of a bismuth-thiol (BT) composition.
10. A method of treating an inflammation-related disease or condition responsive to an increased level of IL-10, comprising administering to a subject diagnosed with an inflammatory condition an effective amount of a bismuth-thiol (BT) composition.
11. A method for increasing IL-10 production in a subject suffering from an inflammation-related disease or condition, comprising administering to the subject an effective amount of a bismuth-thiol composition.
12. A method for elevating the level of IL-10 in a cell, comprising contacting the cell with a bismuth-thiol composition.
13. The method of any one of embodiments 1-12, wherein the BT composition comprises a plurality of particles of a bismuth-thiol (BT) compound suspended therein, wherein the particles have a D90 of less than about 5 μm.
14. The method of any one of embodiments 1-13, wherein the BT composition comprises a plurality of particles of a bismuth-thiol (BT) compound suspended therein, wherein the particles have a D90 of less than about 2 μm and/or the D50 of said particles is less than or equal to about 1.0 μm.
15. The method of any one of embodiments 1-14, wherein the BT compound modulates innate immune activation.
16. The method of any one of the preceding embodiments, wherein the BT compound is selected from the group consisting of BisBAL, BisEDT, Bis-dimercaprol, BisDTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, BisPyr/EDT, Bis-Pyr/PDT, Bis-Pyr/Tol, Bis-Pyr/Ery, bismuth-1-mercapto-2-propanol, and Bis-EDT/2-hydroxy-1-propanethiol.
17. The method of any one of the preceding embodiments, wherein the BT compound is BisEDT or BisBAL.
18. The method of any one of the preceding embodiments, wherein the BT compound is BisEDT.
19. The method of any one of the preceding embodiments, wherein the BT composition further comprises about 0.05% to about 1.0% polysorbate 80, about 0.05 to 40 mM sodium chloride, optionally about 1% to about 10% of methylcellulose, and optionally about 2 to 20 mM sodium phosphate at about pH 7.4.
20. The method of any one of the preceding embodiments, wherein the BT composition is administered three times per day, two times per day, once daily, every other day, once every three days, three times per week, once every week, once every other week, once every month, or once every other month.
21. The method of any one of the preceding embodiments, wherein the subject is administered multiple doses of the BT composition daily or weekly.
22. The method of any one of the preceding embodiments, wherein the subject is administered multiple doses of the BT composition daily or weekly for a length of time ranging from about one week to about 12 weeks.
23. The method of any one of the preceding embodiments, wherein the BT composition is topically administered.
24. A method of treating an inflammation-related disease or condition in a subject in need thereof, comprising administering to the subject an effective amount of a bismuth-thiol (BT) composition that comprises a BT compound, wherein the inflammation-related disease or condition is regulated by or associated with the activation of an inflammasome.
25. The method of embodiment 24, wherein the inflammasome is the NLRP3 inflammasome or the AIM2 inflammasome.
26. The method of embodiment 25, wherein the inflammasome is the NLRP3 inflammasome.
27. The method of any one of embodiments 24-26, wherein upon administration of the BT composition to the subject, the inflammasome expression is inhibited.
28. The method of embodiment 27, wherein the inhibited inflammasome is the NLRP3 inflammasome or the AIM2 inflammasome.
29. The method of embodiment 27 or 28, wherein the inflammasome is inhibited in cells of the subject not having a microbial infection.
30. The method of any one of embodiments 24-29, wherein the administration of the bismuth-thiol (BT) composition comprising a BT compound additionally prevents, reduces, or treats a microbial infection, or prevents, reduces, or treats microbial products in the subject.
31. The method of embodiment 30, wherein the microbial products comprise biofilms and/or superantigens.
32. The method of embodiment 31, wherein the microbial infection in the subject is at least partially located in the inflammasome-expressing cells of the subject.
33. The method of any one of embodiments 27-32, wherein the inflammasome-expressing cells are macrophages, white blood cells, bronchial epithelial cells, alveolar epithelial cells, or combinations thereof.
34. The method of embodiment 33, wherein the inflammasome-expressing cells are macrophages.
35. The method of embodiment 33 or 34, wherein the macrophages are THP-1 macrophages.
36. The method of any one of embodiments 32-34, wherein upon administration of the BT composition to the subject, the microbial infection in the inflammasome-expressing cells is reduced.
37. The method of any one of embodiments 24-36, wherein only cells of the subject that have a microbial infection exhibit increased inflammasome release.
38. The method of embodiment 37, wherein the release of the NLRP3 inflammasome and/or the AIM2 inflammasome is increased.
39. The method of embodiment 37, wherein the release of the NLRP3 inflammasome is increased.
40. The method of any one of embodiments 1-39, wherein the inflammation-related disease or condition is regulated by or associated with the activation of one or more cytokines.
41. The method of embodiment 40, wherein the one or more cytokines is a pro-inflammatory cytokine selected from one of more IFN-γ, MIP-3α, MIP-1β, TNF-α, IL-22, IL-17A, IL-15, and IL-13.
42. The method of any one of embodiments 24-41, wherein the BT compound is selected from BisBAL, BisEDT, Bis-dimercaprol, BisDTT, Bis-2-mercaptoethanol, BisDTE, BisPyr, BisEry, BisTol, BisBDT, BisPDT, BisPyr/Bal, BisPyr/BDT, BisPyr/EDT, BisPyr/PDT, BisPyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.
43. The method of embodiment 42, wherein the BT compound is selected from BisEDT, BisBal, BisPyr, BisEry, BisTol, BisBDT, or BisEDT/2-hydroxy-1-propane thiol.
44. The method of embodiment 42, wherein the BT compound is BisEDT or BisBAL.
45. The method of embodiment 42, wherein the BT compound is BisEDT.
46. The method of any one of embodiments 24-45, wherein the administering of the BT compound decreases expression of NLRP3 and/or AIM2 inflammasomes.
47. The method of embodiment 46, wherein the expression of the NLRP3 and/or AIM2 inflammasomes is decreased by about 50%, about 40%, about 30% about 20%, or about 10%.
48. A method of treating, reducing, or preventing an inflammatory response in a subject in need thereof, comprising administering to the subject an effective amount of a bismuth-thiol (BT) composition that comprises a BT compound.
49. The method of embodiment 48, wherein the transmigration or recruitment of leukocytes in a subject are reduced after administration of the bismuth-thiol (BT) composition to the subject
50. The method of embodiment 48, wherein a site of inflammation in the subject has a decreased number of leukocytes after administration of the bismuth-thiol (BT) composition to the subject.
51. The method of any one of embodiments 48-50, wherein the production of IL-10 in the cell of subject is increased after administration of the bismuth-thiol (BT) composition to the subject.
52. The method of any one of embodiments 48-51, wherein the administration of the BT composition results in a decrease in the secretion of IFN-γ, MIP-3α, MIP-1β, TNF-α, IL-22, IL-17A, IL-15, and IL-13 in the cell of a subject.
53. The method of any one of embodiments 48-51, wherein the administration of the BT composition results in an increase in the secretion of IL-2 in the cell of a subject.
54. The method of any one of embodiments 48-51, wherein the leukocytes are neutrophils.
55. The method of any one of embodiments 48-54, wherein the BT compound is selected from BisBAL, BisEDT, Bis-dimercaprol, BisDTT, Bis-2-mercaptoethanol, BisDTE, BisPyr, BisEry, BisTol, BisBDT, BisPDT, BisPyr/Bal, BisPyr/BDT, BisPyr/EDT, BisPyr/PDT, BisPyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.
56. The method of embodiment 55, wherein the BT compound is selected from BisEDT, BisBal, BisPyr, BisEry, BisTol, BisBDT, or BisEDT/2-hydroxy-1-propane thiol.
57. The method of embodiment 56, wherein the BT compound is BisEDT or BisBAL.
58. The method of embodiment 57, wherein the BT compound is BisEDT.
59. The method of any one of embodiments 48-58, wherein the BT composition comprises a plurality of particles of a bismuth-thiol (BT) compound suspended therein, wherein the particles have a D90 of less than about 5 μm.
60. The method of embodiment 59, wherein the BT composition comprises a plurality of particles of a bismuth-thiol (BT) compound suspended therein, wherein the particles have a D90 of less than about 2 μm.
61. The method of embodiment 59 or 60, wherein the D50 of said particles is less than or equal to about 1.0 μm.
62. The method of any one of embodiments 1-47, wherein the administration of the BT composition to a subject results in a decrease in the secretion of one or more pro-inflammatory cytokines.
63. The method of any one of embodiments 1-47 and 62, wherein the administration of the BT composition to a subject results in a decrease in the secretion of IFN-γ, MIP-3α, MIP-1β, TNF-α, IL-22, IL-17A, IL-15, and/or IL-13 compared to a subject not administered the BT composition.
64. The method of any one of embodiments 1-47 and 62, wherein the administration of the BT composition to a subject results in a decrease in the secretion of MIP-3α, MIP-1β, and/or TNF-α compared to a subject not administered the BT composition.
65. The method of any one of embodiments 1-47 and 62, wherein the administration of the BT composition to a subject results in a decrease in the secretion of TNF-α compared to a subject not administered the BT composition.
66. The method of any one of embodiments 63-65, wherein the secretion of IFN-γ, MIP-3α, MIP-1β, TNF-α, IL-22, IL-17A, IL-15, and/or IL-13 is decreased by about 2-fold, about 3-fold, about 4-fold, about 5-fold, about 6-fold, about 7-fold, about 8-fold, about 9-fold, or about 10-fold compared to a subject not administered the BT composition.
67. The method of any one of embodiments 63-65, wherein the secretion of IFN-γ, MIP-3α, MIP-1β, TNF-α, IL-22, IL-17A, IL-15, and/or IL-13 is decreased by about 3-fold compared to a subject not administered the BT composition.
68. The method of any one of embodiments 1-47, wherein the administration of the BT composition to a subject results in an increase in the secretion of one or more anti-inflammatory cytokines.
69. The method of any one of embodiments 1-47 and 68, wherein the administration of the BT composition to a subject results in an increase in the secretion of IL-2 compared to a subject not administered the BT composition.
70. The method of embodiment 69, wherein the secretion of IL-2 is increased by about 2-fold, about 3-fold, about 4-fold, about 5-fold, about 6-fold, about 7-fold, about 8-fold, about 9-fold, or about 10-fold compared to a subject not administered the BT composition.
71. The method of embodiment 69, wherein the secretion of TL-2 is increased by about 3-fold compared to a subject not administered the BT composition.
72. The method of any one of embodiments 1-47 and 68, wherein the administration of the BT composition to a subject results in an increase in the secretion of IL-10 compared to a subject not administered the BT composition.
73. The method of embodiment 69, wherein the secretion of IL-10 is increased by about 2-fold, about 3-fold, about 4-fold, about 5-fold, about 6-fold, about 7-fold, about 8-fold, about 9-fold, or about 10-fold compared to a subject not administered the BT composition.
74. The method of embodiment 69, wherein the secretion of IL-10 is increased by about 3-fold compared to a subject not administered the BT composition.
75. The method of any one of embodiments 1-74, wherein the effective amount of the BT composition is effective to increase IL-10 production in the subject.
76. The method of any one of embodiments 1-75, wherein the effective amount of the BT composition is effective to decrease IFN-γ, MIP-3α, MIP-1β, TNF-α, IL-22, IL-17A, IL-15, and/or IL-13 production in the subject.
77. The method of any one of embodiments 1-76, wherein the effective amount of the BT composition is effective to inhibit neutrophil-mediated inflammation, transmigration, and/or infiltration in the subject.
78. The method of any one of embodiments 24-77, wherein the BT composition is topically administered.

### EXAMPLES

The following examples are provided to illustrate the present disclosure and should not be construed as limiting thereof. Additional experimental procedures and details can be found in International Patent Application Nos. WO/2010/091124, WO/2011/097347, WO/2012/021754, WO/2020/028558, and WO/2020/028561, which are hereby incorporated by reference in their entireties for all purposes.

### Example 1: General Synthesis of Pravibismane

The starting materials and reagents used in preparing these compounds are either available from commercial supplier such as Aldrich Chemical Co., Bachem, etc., or can be made by methods well known in the art. The starting materials and the intermediates and the final products of the reaction can be isolated and purified if desired using conventional techniques, including but not limited to filtration, distillation, crystallization, chromatography, and the like and can be characterized using conventional means, including physical constants and spectral data. Unless specified otherwise, the reactions described herein take place at atmospheric pressure over a temperature range from about −78° C. to about 150° C.

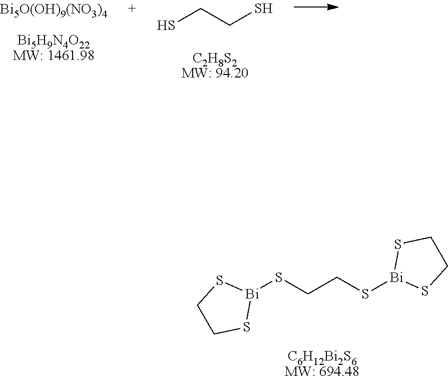

Bismuth-1,2-ethanedithiol particles (BisEDT, pravibismane, CAS number 175880-68-9) were prepared as follows: To an excess (11.4 L) of 5% aqueous HNO₃ at room temperature in a 15 L polypropylene carboy was slowly added by dropwise addition 0.331 L (˜0.575 moles) of an aqueous Bi(NO₃)₃ solution (43% Bi(NO₃)₃ (w/w), 5% nitric acid (w/w), 52% water (w/w), Shepherd Chemical Co., Cincinnati, OH, product no. 2362; δ˜1.6 g/mL) with stirring, followed by slow addition of absolute ethanol (4 L). Some white precipitate formed but was dissolved by continued stirring. An ethanolic solution (˜1.56 L, ˜0.55 M) of 1,2-ethanedithiol (CAS 540-63-6) was separately prepared by adding, to 1.5 L of absolute ethanol, 72.19 mL (0.863 moles) of 1,2-ethanedithiol using a 60 mL syringe, and then stirring for five minutes. The 1,2-ethanedithiol/EtOH reagent was then slowly added by dropwise addition over the course of five hours to the aqueous Bi(NO₃)₃/HNO₃ solution, with continued stirring overnight. The formed product was allowed to settle as a precipitate for approximately 15 minutes, after which the filtrate was removed at 300 mL/min using a peristaltic pump. The product was then collected by filtration on fine filter paper in a 15-cm diameter Buchner funnel, and washed sequentially with three, 500-mL volumes each of ethanol, USP water, and acetone to obtain BisEDT (694.51 gm/mole) as a yellow amorphous powdered solid. The product was placed in a 500 mL amber glass bottle and dried over CaCl₂ under high vacuum for 48 hours. Recovered material (yield ˜200 g) gave off a thiol-characteristic odor. The crude product was redissolved in 750 mL of absolute ethanol, stirred for 30 min, then filtered and washed sequentially with 3×50 mL ethanol, 2×50 mL acetone, and washed again with 500 mL of acetone. The rewashed powder was triturated in 1M NaOH (500 mL), filtered and washed with 3×220 mL water, 2×50 mL ethanol, and 1×400 mL acetone to afford 156.74 gm of purified BisEDT. Subsequent batches prepared in essentially the same manner resulted in yields of about 78-91%.

The product was characterized as having the structure shown above by analysis of data from ¹H and ¹³C nuclear magnetic resonance (NMR), infrared spectroscopy (IR), ultraviolet spectroscopy (UV), mass spectrometry (MS) and elemental analysis. An HPLC method was developed to determine chemical purity of BisEDT whereby the sample was prepared in DMSO (0.5 mg/mL). The λₘₐₓ was determined by scanning a solution of BisEDT in DMSO between 190 and 600 nm. Tsocratic HPLC elution at 1 mL/min was performed at ambient temperature in a mobile phase of 0.1% formic acid in acetonitrile:water (9:1) on a Waters (Millipore Corp., Milford, MA) model 2695 chromatograph with UV detector monitoring at 265 nm (λₘₐₓ), 2 μL injection volume, equipped with a YMC Pack PVC Sil NP, 5 μm, 250×4.6 mm inner diameter analytical column (Waters) and a single peak was detected, reflecting chemical purity of 100±0.1%. Elemental analysis was consistent with the structure of BisEDT as shown above.

The dried particulate matter was characterized to assess the particle size properties. Briefly, particles were resuspended in 2% Pluronic® F-68 (BASF, Mt. Olive, NJ) and the suspension was sonicated for 10 minutes in a water bath sonicator at standard setting prior to analysis using a Nanosizer/Zetasizer Nano-S particle analyzer (model ZEN1600 (without zeta-potential measuring capacity), Malvern Instruments, Worcestershire, UK) according to the manufacturer's recommendations. From compiled data of two measurements, particles exhibited a unimodal distribution with all detectable events between about 0.6 microns and 4 microns in volumetric mean diameter (VMD) and having a peak VMD at about 1.3 microns.

### Example 2: Preparation of Microparticulate Bismuth-1-2-Ethanedithiol (BisEDT, Pravibismane)

Particulate bismuth-1,2-ethanedithiol (BisEDT, pravibismane) was prepared as follows: Water (25.5 L) and 70% nitric acid (1800 mL) were mixed together in a Nalgene reactor. Then, water (2300 mL) was added to an Erlenmeyer flask, followed by bismuth subnitrate (532 g), and the mixture was stirred. To the mixture was added 70% nitric acid (750 mL) to obtain a clear solution. This solution was transferred into the Nalgene reactor and the resulting mixture was stirred for 20 min. Then, 9.5 L of 95% EtOH was added to the reactor in three portions.

Separately, 1,2-ethanedithiol, 98%, (229 mL) was added to a bottle followed by two 250 mL EtOH portions with stirring. A further 5 L EtOH was added to the bottle with stirring. The 1,2-ethanedithiol solution was then added to the reactor over about 4 hours while stirring. After stirring for 18 hours, the solids were allowed to settle for 2 hours. EtOH (20 L) was added and the mixture stirred for 24 hours. The solids were allowed to settle for 1.5 hours, then separated by filtration of the mixture, followed by rinsing with EtOH.

To the empty reactor was added 9 L EtOH and the filtered solids, which was stirred for 18 hours. The solids were allowed to settle for 1 hour, then separated by filtration of the mixture, followed by rinsing with EtOH. Next, the empty reactor was charged with 9 L acetone, 99.5%, and the filtered solids, which was stirred for 15 hours. The solids were allowed to settle for 1.5 hours, then separated by filtration of the mixture, followed by rinsing with acetone. Again, the empty reactor was charged with 9 L acetone, 99.5%, and the filtered solids, which was stirred for 1.4 hours. The solids were filtered and air-dried for 69 hours, then vacuum-dried for 4 hours. After mixing the solid, it was sieved through a 10 mesh (2 mm) and then 18 mesh (1 mm) sieve to give BisEDT.

### Example 3: Studies on Processing Conditions on BisEDT Particle Size Distribution

It was observed that careful control of the reaction temperature and the rate of 1,2 ethanedithiol addition had pronounced impact on the BisEDT particle size distribution. Representative syntheses are shown below for BisEDT synthesized at 20° C. with a 1.25 hour addition of 1,2-ethane via syringe pump and BisEDT synthesized at 15° C. with a 1 hour addition of 1,2-ethane via syringe pump. Table 1 below shows that temperature conditions play a critical role in particle size distribution, with processing temperatures in the range of 20-30° C. providing BisEDT particle size distribution that are both small and uniform in particle size (such as a D90 below 2 microns).

Representative synthesis of BisEDT at 20° C. with 1.25 hour addition of thiol via syringe pump, and polypropylene cloth for filtration BisEDT synthesis was performed on 10-g scale. To a 1-L jacketed reactor was charged USP water (480 mL, 48 vol), followed by 70% HNO₃ (34 mL, 3.4 vol). A solution of bismuth subnitrate (10 g, 6.84 mmol) in water (43 mL, 4.3 vol) and 70 HNO₃ (14 mL, 1.4 vol) was added at 20° C. The reaction mixture was cooled to 15° C. for addition of 95% Ethanol. The 95% ethanol (180 mL, 18 vol) was then added slowly. (Ethanol addition is exothermic, temperature reached 22° C.). The temperature was then adjusted back to 20° C. This was followed by dropwise addition of 1,2 ethanedithiol (4.3 mL, 7.5 mmol in 95% ethanol in 94 mL, 9.4 vol) over a period of 1.25 hour with the batch temperature at 20° C. during which time it turned into a yellow suspension. The reaction was stirred at 20° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.81 g 91.0%).

Representative synthesis of BisEDT at 15° C. with 1 hour addition of thiol via syringe pump, and polypropylene cloth for filtration: The synthesis BisEDT was performed on 10-g scale, temperature profile was studied with data logger. Ethane dithiol was added at 15° C. over 1 hour via syringe pump and the filtration was performed using PP filter cloth. To a 1-L jacketed reactor was charged USP water (480 mL, 48 vol) and cooled to 15° C., followed by 70% HNO₃ (34 mL, 3.4 vol). A solution of bismuth subnitrate (10 g, 6.84 mmol) in water (43 mL, 4.3 vol) and 70% HNO₃ (14 mL, 1.4 vol) was added at the same temperature. The 95% ethanol (180 mL, 18 vol) was then added slowly. (Ethanol addition is exothermic, temperature reached 22.5° C.). It was then allowed to cool to 15° C. This was followed by dropwise addition of 1,2 ethanedithiol (4.3 mL, 7.5 mmol in 95% ethanol in 94 mL, 9.4 vol) over an hour with the batch temperature at 15° C. The reaction was allowed to stir at 15° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and dried at 45° C. (18 hours) to provide canary yellow solid (10.43 g 87.8%).

TABLE 1
Particle Size Distribution of BisEDT samples

| Sample | D(10) μm | D(50) μm | D(90) μm | D[4, 3] μm | Conditions |
|---|---|---|---|---|---|
| 1 | 0.80 | 2.4 | 5.9 | 2.9 | Dalton Synthesis Conditions |
| 2 | 0.58 | 1.7 | 3.9 | 2.0 | Dalton Synthesis Conditions |
| 3 | 0.59 | 1.9 | 4.5 | 2.3 | 30° C., 5 h addition of 1,2-ethane dithiol |
| | | | | | via addition funnel |
| 4 | 0.44 | 1.48 | 3.1 | 1.7 | 30° C., 4 hour addition of 1,2-ethane |
| | | | | | dithiol via syringe pump |
| 5 | 0.33 | 0.72 | 1.6 | 0.86 | 20° C., 1 h addition of 1,2-ethane dithiol |
| | | | | | via addition funnel |
| 6 | 0.34 | 0.87 | 1.8 | 0.98 | 20° C., 4 h addition of 1,2-ethane dithiol |
| | | | | | via addition funnel |
| 7 | 0.39 | 1.3 | 1.6 | 1.4 | 20° C., 1 hour addition of 1,2-ethane |
| | | | | | dithiol via syringe pump. Sample |
| | | | | | slurried in EtOH. Cloth filtration |
| 8 | 0.36 | 1.0 | 1.8 | 1.0 | 20° C., 1 hour addition of 1,2-ethane |
| | | | | | dithiol via syringe pump. Sample |
| | | | | | slurried in MeOH. Cloth filtration |
| 9 | 0.67 | 1.0 | 1.9 | 1.1 | 20° C., 1 hour addition of 1,2-ethane |
| | | | | | dithiol via syringe pump. Sample |
| | | | | | slurried in Abs. MeOH. Cloth filtration |
| 10 | 0.36 | 0.88 | 1.7 | 0.97 | 20° C., 1 hour addition of 1,2-ethane |
| | | | | | dithiol via syringe pump. Sample |
| | | | | | slurried in IPA. Cloth filtration |
| 11 | 0.38 | 1.2 | 2.4 | 1.4 | 15° C. 1.5 hour addition of 1,2-ethane |
| | | | | | dithiol via syringe pump. Cloth filtration |
| 12 | 0.37 | 1.2 | 2.4 | 1.3 | 20° C., 1.25 hour addition of 1,2-ethane |
| | | | | | via syringe pump |
| 13 | 0.36 | 0.98 | 2.1 | 1.2 | 10° C., 1 h addition of 1,2-ethane dithiol |
| | | | | | via syringe pump |
| 14 | 0.36 | 1.0 | 2.1 | 1.2 | 10° C. 1 hour addition of 1,2-ethane dithiol |
| | | | | | via syringe pump. Cloth filtration |
| 15 | 0.32 | 0.72 | 1.6 | 0.86 | 10° C., 4 hours addition of 1,2-ethane |
| | | | | | dithiol via syringe pump. Cloth filtration. |

### Example 4: Quantification of Inflammasome Expression in Infected and Uninfected Macrophages Treated with BisEDT

### RT-qPCR of NLRP3 and AIM2 Inflammasomes

The ability of pravibismane to influence the expression of NLRP3 and AIM2 inflammasomes was evaluated using RT-qPCR and compared to control.

### Procedure

THP-1 macrophages were differentiated with 50 ng/ml PMA for 24 hours, followed by 24 hours in media prior to infection. Differentiated THP-1 macrophages were infected with M. avium or M. abscessus for 1 hour with an MOI of 5. Infected cells were washed twice, followed by 1 hour of antibiotic treatment with 200 μg/ml of amikacin to remove extracellular bacteria. After the antibiotic treatment step, cells were washed once more and BisEDT (BIZ) was added at the indicated concentrations. The treatments (see Table 2) included: (1) uninfected, (2) uninfected+range of BisEDT, (3) untreated+infected with M. avium, (4) M. avium infected+BIZ, (5) untreated+M. abscessus, and (6) M. abscessus infected+BIZ. The time points for sample preparation were 48 hours post infection. CFUs were determined as described above and RNA was collected from similar experiments.

TABLE 2
Schema of THP-1 Cells, Infections and Treatments¹

| | | |
|---|---|---|
| Uninfected | M. avium infected, untreated | M. abscessus infected, |
| | | untreated |
| Uninfected + 0.5 μg/ml BIZ | M. avium infected, + 2 μg/ml | M. abscessus infected, + 0.5 |
| | BIZ | μg/ml BIZ |
| Uninfected + 1 μg/ml BIZ | M. avium infected, + 4 μg/ml | M. abscessus infected, + 1 |
| | BIZ | μg/ml BIZ |
| Uninfected + 2 μg/ml BIZ | M. avium infected, pre-treated | M. abscessus infected, pre- |
| | with 2 μg/ml BIZ² | treated with 0.5 μg/ml BIZ² |
| Uninfected + 4 μg/ml BIZ | M. avium infected, pre-treated | M. abscessus infected, pre- |
| | with 4 μg/ml BIZ² | treated with 1 μg/ml BIZ² |

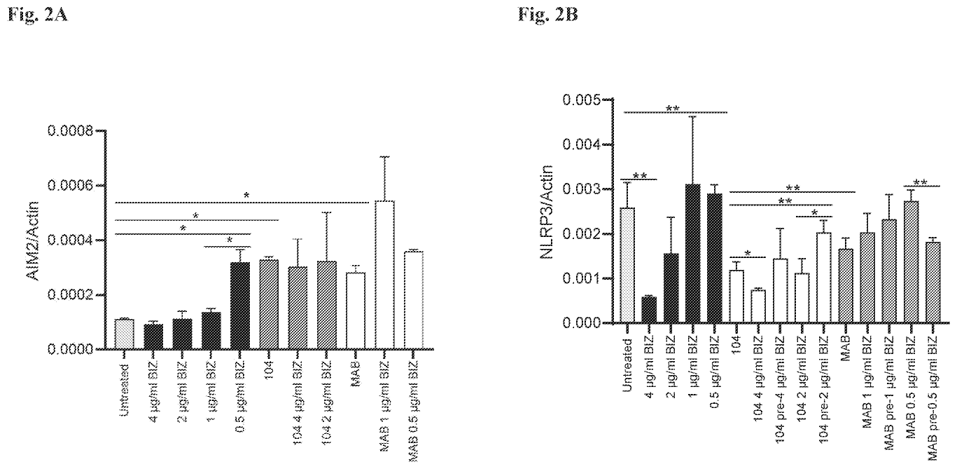

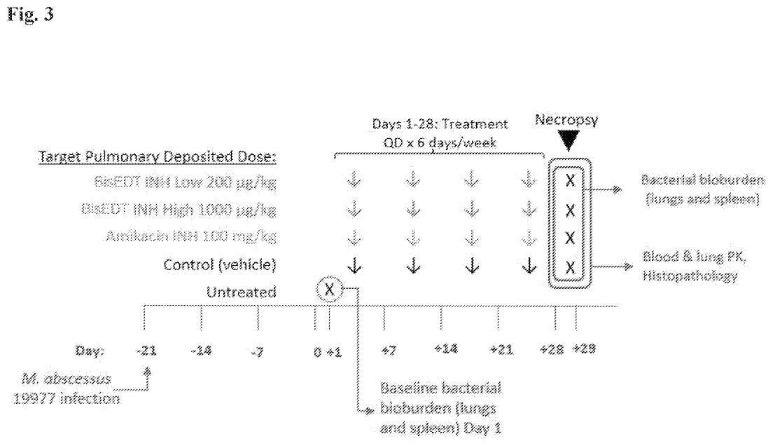

¹Results summarized in FIG. 2A and FIG. 2B.
²Results summarized in FIG. 2B.

THP-1 RNA from the cells was isolated using the Rneasy mini kit (Qiagen), followed by treatment with Dnase 1 recombinant (Roche Diagnostics) for 1 hour at 37° C. to remove contaminating genomic DNA. The DNase was inactivated with Turbo DNase-inactivation reagent (Turbo DNA-free kit, Thermo Fisher Scientific) for 2 min at 37° C. Inactivation reagent was removed via centrifugation for 1 min at 10,000×g, and RNA transferred to new collection tubes. RNA samples were stored at −4° C. for future processing. cDNA was transcribed from host RNA using the iScript cDNA synthesis kit (Bio-Rad). The quality of cDNA was tested by PCR with Gold 360 master mix using the manufacturer's specifications (Thermo Fisher Scientific). The RT-qPCR reaction was performed using iQ SYBR Green Supermix (Bio-Rad) and an iCycler (CFX Connect Real-Time Systems, Bio-Rad) as previously described (1). Standard curves for each gene were run in each reaction and used to calculate absolute values of mRNA. Gene expression data are presented as relative expression to beta-actin. Primers (Table 3) were designed in Primer3 using sequences from GenBank (National Center for Biotechnology Information).

TABLE 3
Primers used for RT-qPCR

| | |
|---|---|
| AIM2 Forward | 5′-CAACGTGCTGCACCAAAAGT-3′ |
| AIM2 Reverse | 5′-GCTTGCCTTCTTGGGTCTCA-3′ |
| NLRP3 Forward | 5′-CTTCTCTGATGAGGCCCAAG-3′ |
| NLRP3 Reverse | S′-GCAGCAAACTGGAAAGGAAG-3′ |
| Beta-actin Forward | S′-CATGTACGTTGCTATCCAGGC-3′ |
| Beta-actin Reverse | 5′-CTCCTTAATGTCACGCACGAT-3′ |

### Results

AIM2 gene expression was determined after THP-1 macrophages were treated with BisEDT and/or infected with mycobacteria species (FIG. 2A). Untreated macrophages determined background levels of gene expression. Treatment of macrophages with BisEDT and without infection showed a dose dependent response to the compound. The higher the dose, the more AIM2 gene expression was reduced, acting as anti-inflammatory. When macrophages were infected with either M. avium or M. abscessus, AIM2 expression increased compared to untreated control, indicating a proinflammatory response. Combining BisEDT with infected cells did not change the gene expression in M. avium cells, while an even greater increase of expression can be seen with M. abscessus treated with 1 μg/ml BisEDT. This strain dependent difference may be due to the killing of M. abscessus allowing for the AIM2 pathway to function further.

BisEDT has a dose-related inhibitory effect on NLRP3 inflammasome expression in uninfected cells. In order to determine if the inhibitory effect on the inflammatory response could be maintained by treating macrophages with BisEDT prior to the infection, macrophage monolayers were treated with 4 concentrations of BisEDT, and then infected with either M. avium 104 or M. abscessus 19977 (FIG. 2B). The expression of NLRP3 was higher in M. avium infected cells pre-treated with 2 μg/ml BisEDT compared to M. avium only infected cells. In M. abscessus infected cells, NLRP3 expression remained unchanged, except NLRP3 expression was reduced in cells pretreated with 0.5 μg/ml BisEDT compared to M. abscessus cells treated with 0.5 μg/ml BisEDT. However, when BisEDT was used after the infections, the inhibitory effect was not observed.

### CONCLUSIONS

BisEDT inhibits AIM2 and NLRP3 inflammasome expression of uninfected BisEDT treated macrophage cells in a dose-dependent manner, while an even greater dose-related NLRP3 inhibition by BisEDT was observed in NTM-infected macrophages.

In case of MAH 104 infection, 4 μg/ml BisEDT treatment reduced NLRP3 expression compared to MAH 104 infected cells. MAH 104 cells pre-treated with 2 μg/ml BisEDT resulted in higher NLRP3 expression compared to MAH 104 infected cells.

When added prior to MAB 19977 infection (pre-treatment), 1 μg/ml BisEDT did not impact NLRP3 expression, but pretreatment with 0.5 μg/ml BisEDT reduced NLRP3 expression.

### Example 5: Efficacy of BisEDT and Reduction of Inflammation in a Chronic M. abscessus Infection in Mice

### BACKGROUND

BisEDT is a novel bioenergetic inhibitor antibiotic that affects the energy flow in the bacterial membrane which prevents ATP production. This mechanism of action is becoming increasingly popular for new antibiotics. Our purpose is to evaluate the efficacy of BisEDT through inhalation against pulmonary M. avium and M. abscessus infection in a mouse model. Since 2012, the collection of small molecule inhibitors of bioenergetics has expanded dramatically. The inhibitors have now become a major component (>30%) of all new antimycobacterial drugs in clinical trials and are included in more than 65% of Phase III trial regimens. Bioenergetic inhibitors have been minimally explored for efficacy in NTM infection and can act as an alternative for resistant NTM infections. These formulations can improve the outcome of patient clearance to previously resistant strains of NTM.

Inhalation distribution of antimicrobials is becoming the preferred application for lung infection treatment. The benefits of inhalation include site specific drug targeting which avoids systematic administration, higher therapeutic concentrations at the site of infection, and reduces off target effects and some toxicity. Earlier work on rats had shown high tolerability at doses as high as 59 μg/kg/day for the duration of 28 days without adverse effects on the animals. This research is aimed at identifying a new treatment for pulmonary NTM, which will minimize the discomfort and cost of care of affected individuals, and possibly also shorten the duration of the infection.

### Study Design

In vivo studies were conducted in mice to separately model chronic M. abscessus infection. The animals were treated for 28 days and grouped according to dosage: vehicle, low dose BisEDT, high dose BisEDT, and amikacin. At the end of treatment, blood, lungs were collected for analysis. Because BisEDT showed strong efficacy against M. abscessus in vivo, further work was done to investigate the propensity of the bacteria to develop resistance, both naturally and from strains collected from mouse lungs. Histopathology was conducted on lung samples from the in vivo studies.

M. abscessus Infection Protocol

A total of sixty female SCID/Beige mice between 6-8 weeks of age were used. Mice were placed up to 4 animals per cage and allowed 1 week of acclimation prior to infection.

Inoculums of M. abscessus strain 19977 (MAB) used in this study were made from highly virulent frozen stocks with low passage numbers. Bacteria were allowed to grow to log phase after 4 days on 7H10 media agar plates at 37° C. Once confluent, inoculums were made by suspending bacteria at 10⁹ in Hank's balanced salt solution (HBSS) as determined by optical density (O.D.) and further quantified by serial dilution to determine the CFU/mL of suspension. At this higher concentration of bacteria, only 20 μL of inoculum was necessary to deposit 10⁸ bacteria per mouse.

### Anesthetization

On the day of infection mice were anesthetized with isoflurane by the drop jar method. Mice were scruffed in a standard one-handed grip and the nose of the mouse is held of a tube containing gauze containing a small amount of isoflurane. Mice were monitored for breathing and once rapid breaths have slowed down initially the mouse is removed from direct anesthesia and 20 μL of bacterial suspension was deposited on the left nostril. The mice then slowly breath in the droplet. Each mouse was then held in hand until complete consciousness was obtained and then the mouse was replaced into its home cage. After infection was initiated, 3 weeks were allowed to pass in order to allow the bacteria to establish a chronic lung pathology for eventual treatment.

After establishing each infection, 12 mice were euthanized the day before treatment began to determine the baseline bacterial load. Mice were euthanized using CO₂ in accordance with the recommendations of the Guide for the Care and Use of Laboratory Animals (NRC 2011). Lungs were collected and homogenized then serial diluted for CFU enumeration.

### Drug Inhalation Protocol

A nose-only inhalation exposure chamber from CH technologies was used for this experiment. Mice were split into four treatment groups, Vehicle (buffer containing no BisEDT), amikacin (100 μg/kg/day), BisEDT “low” (200 μg/kg/day), and BisEDT “high” (1000 μg/kg/day). Each treatment group consisted of 12 mice. Ten of these mice were used for CFU determination and two mice are used for histology/pathology/lung pharmacokinetics. Treatment was conducted 6 days a week over the course of 28 days total. To create the correct level of deposited dose of each drug tested, 10 mL of test article were made. Amikacin in HBSS at 20 mg/mL was made and stored at 4° C. until needed. BisEDT “high” at a concentration of 8 mg/mL was made once a week and stored at room temperature. BisEDT “low” was diluted from the 8 mg/mL to 1.6 mg/mL the day of treatment. Each treatment used an LC star reusable nebulizer from Pari. Animals were restrained and placed on the inhalation apparatus. The respiratory hose was connected to the inhalation apparatus and a compressor was connected to the nebulizer. The rate of air flow from the compressor to the nebulizer remained at 8.5 L/minute, which generated a nebulized fog that each mouse breathed in over 45 min.

### Sample and Data Collection

After 28 days of treatment with BisEDT, animals were euthanized by CO₂ and secondary cardiac puncture to collect whole blood for quantitation of BisEDT concentration. Lungs and spleens from the CFU mice were homogenized with 1:1 H₂O:Dey-Englev neutralizing broth to deactivate BisEDT. CFUs were determined after serial dilution. Mice for histology and the lung pair split, one half in formalin the other half weighed and snap frozen in liquid N₂ to determine tissue concentration of BisEDT.

Samples for histology were processed by the Veterinary Diagnostic Laboratory. Slides were stained with hematoxylin-eosin (HE stain) for general pathology as well as a slide for acid-fast stain to highlight the mycobacterial infection in lung tissue.

The dosage groups and study schedule for the M. abscessus in vivo study are summarized in Table 4 and FIG. 3.

TABLE 4
M. abscessus infection in SCID mice study overview.

| | | | | Target | | | Mice | | | |
|---|---|---|---|---|---|---|---|---|---|---|
| | | | Target | Pulmonary | | | | Day 1 | | Day 29 |
| | | Inhalation | Presented | Deposited | Formulation | Volume/ | | Baseline | Day 29 | Lung & Blood |
| | Test | Dosing | Dose | Dose | Conc. | Delivery | Female | CFU | CFU | PK/Histo |
| Group | Substance | Regimen | (μg/kg/day) | (μg/kg/day) | (mg/mL) | Time | (n) | (n) | (n) | (n) |
| 1 | N/A | none | N/A | N/A | NA | N/A | 12 | 12 | ns | ns |
| | (Baseline) | | | | | | | | | |
| 2 | Vehicle | 5 days/week, | 0 | 0 | 0 | 10 mL/ | 12 | ns | 10 | 2 |
| | | Days 1-28 | | | | 45 min | | | | |
| 3 | BisEDT | 5 days/week, | 200 | 20 | 1.6 mg/ml | 10 mL/ | 12 | ns | 10 | 2 half lungs for |
| | (low) | Days 1-28 | | | | 45 min | | | | PK, 2 half lungs |
| | | | | | | | | | | for Histo |
| 4 | BisEDT | 5 days/week, | 1,000 | 100 | 8 mg/ml | 10 mL/ | 12 | ns | 10 | 2 half lungs for |
| | (high) | Days 1-28 | | | | 45 min | | | | PK, 2 half lungs |
| | | | | | | | | | | for Histo |
| 5 | Amikacin | 5 days/week, | 100,000 | 10,000 | 20 mg/ml | 10 mL/ | 12 | ns | 10 | 2 |
| | | Days 1-22 | | | | 45 min | | | | |

N/A—Not applicable
ns—Not sampled

Results of M. abscessus Chronic Lung Infection Study

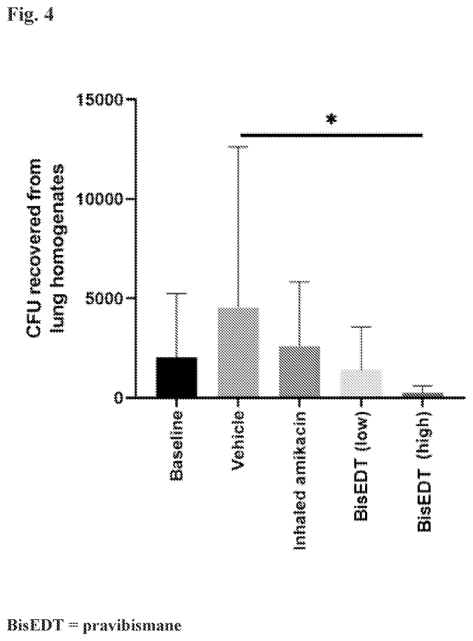

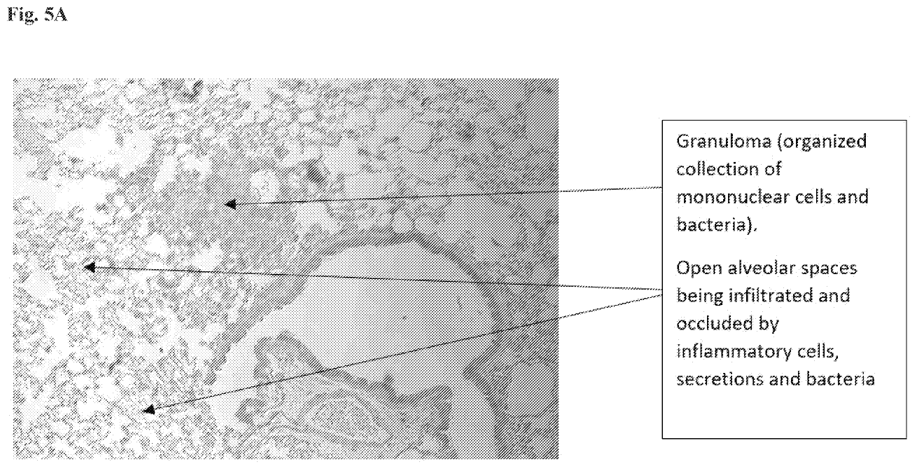

BisEDT doses were well tolerated by the mice and the mice were responsive and within the normal weight range at the end of treatment. Data for CFU recovery from lung and spleen homogenates is provided in FIG. 4. Using a Kruskal-Wallis test, the lung CFU data showed a dose-dependent response to BisEDT, as well as a statistically significant difference (p-value=0.0163) between the high-dose group and the vehicle control.

As shown in FIG. 4 by the reduction of CFU count in lung homogenates, administration of 200 μg/kg of BisEDT (low) in mice infected with M. abscessus provided a bacteriostatic effect. BisEDT is bactericidal at 1000 μg/kg (high) delivered to the lungs of mice infected with against M. abscessus. In the macrophage system it was also very active (bactericidal) against all three clinical strains tested. Moreover, the activity against M. abscessus in this mouse model suggests that BisEDT is active against M. abscessus in biofilm phenotype.

### Histopathology

Histomorphologic Observations from Mice Infected with M. abscessus

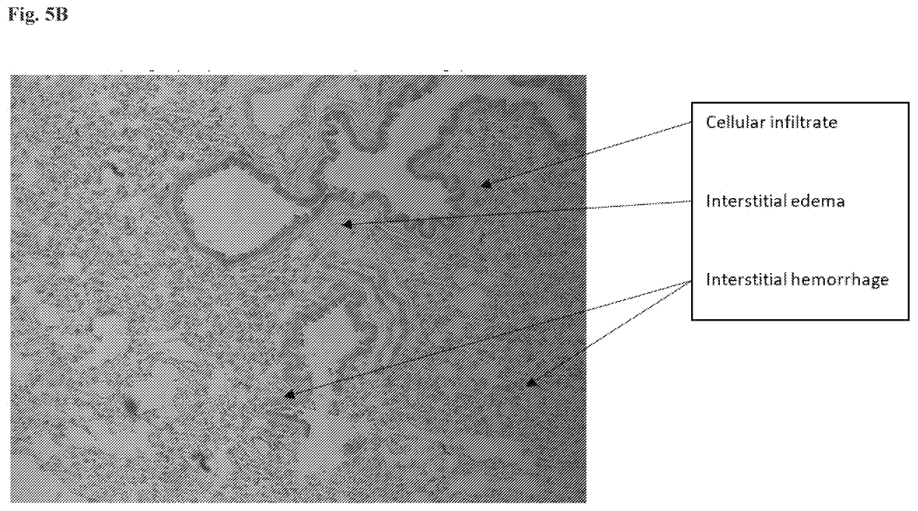

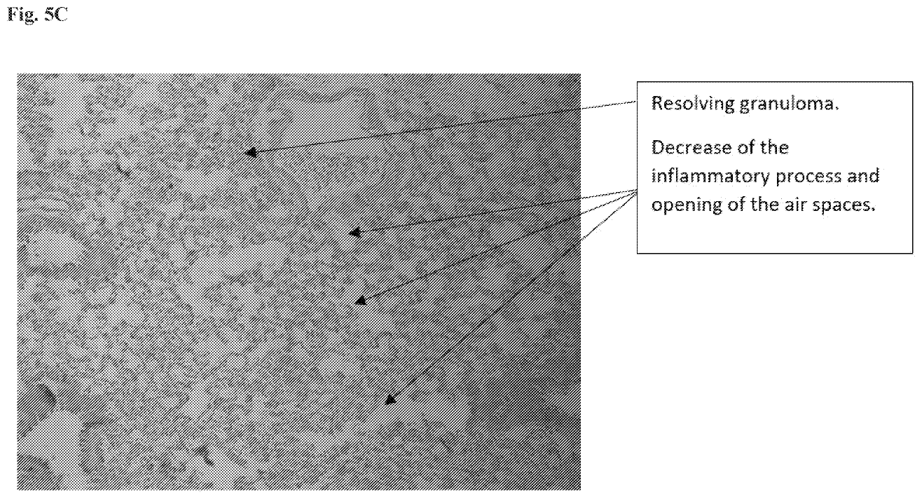

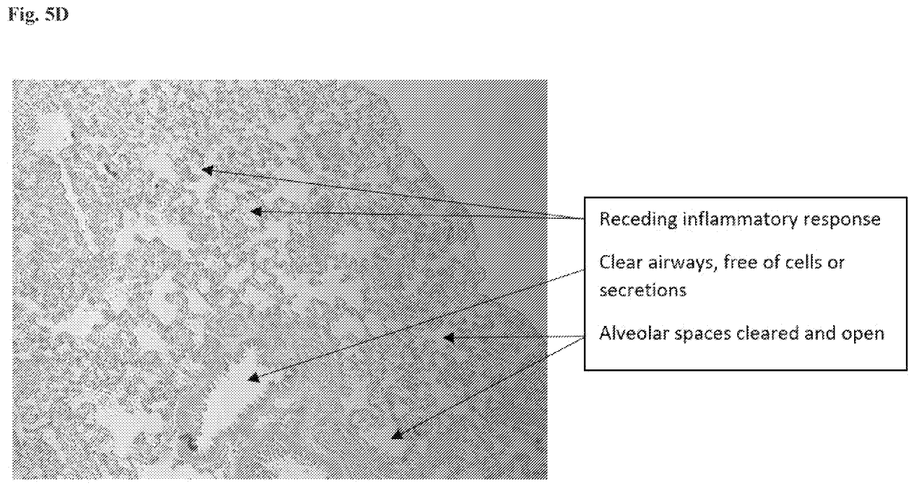

An example of lung tissue from vehicle-treated M. abscessus infected mice is provided in FIG. 5A. As shown in the micrograph, there was indication of inflammatory infiltration of mononuclear cells, bacteria and secretions.

An example of lung tissue from amikacin-treated M. abscessus infected mice is provided in FIG. 5B. As shown in the micrograph, there was indication of infiltration of mononuclear phagocytes, interstitial edema, and interstitial hemorrhage.

An example of lung tissue from low-dose BisEDT-treated M. abscessus infected mice is provided in FIG. 5C. As shown in the micrograph, there was a decrease of inflammatory infiltrate compared to untreated control and a decrease in hemorrhage. Resolving granulomas were also observed.

An example of lung tissue from high-dose BisEDT-treated M. abscessus infected mice is provided in FIG. 5D. As shown in the micrograph, the inflammatory response was receding and clear airways were observed. Red cells that can be from the inflammation are more likely due to the cut of the lung tissue. Note, the alveolar spaces (air spaces) were clean.

Conclusions: the pathology findings support the microbiologic observations. In mice infected with M. abscessus, there was a significant reduction of the disease process, with disappearance of inflammatory cells and opening of alveolar spaces. In mice infected with M. abscessus and treated with BisEDT, there was no indication of an inflammatory process caused by the BisEDT. There was a clear regression of the inflammatory process caused by the infection. Air spaces, both conducting airways and alveolar spaces were opened with very little residual inflammatory process. The granulomas observed were in the dynamic process of resolution. No difference was seen in lungs of treated versus untreated mice suggesting that there was no inflammatory reaction to the BisEDT.

### Example 6: IL-10 Expression in A549 Cells (Human Alveolar Epithelial Cells) Treated with BisEDT

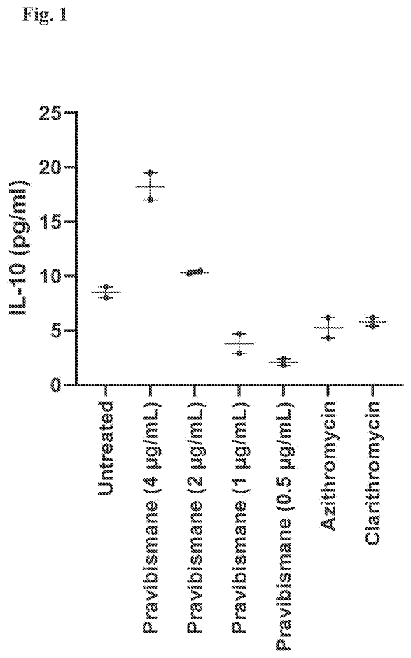

A549 cells were differentiated with 50 ng/mL PMA for 24 hours, followed by 24 hours in media prior to infection. Cells were washed with media and treated with insoluble BisEDT or other antibiotics, and incubated for 24 h post treatment. Cell media was then removed and frozen at −80° C. Samples were briefly vortexed before conducting ELISA analysis for IL-10 expression (FIG. 1). BisEDT at a concentration of 2 μg/mL and 4 μg/mL increased the level of IL-10 expression level in A549 cells compared to untreated control.

### Example 7: Demonstrating Anti-Inflammatory Properties of BisEDT (Pravibismane) In Vitro

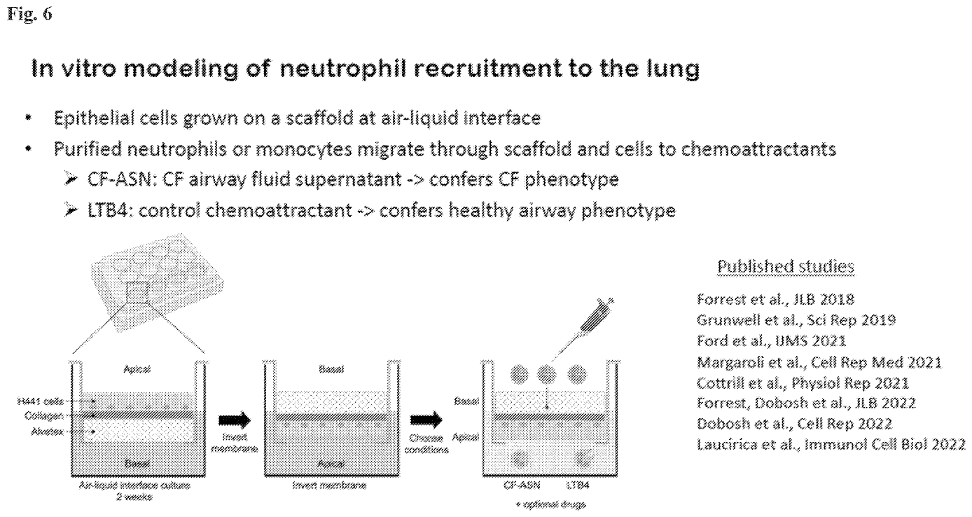

### In Vitro Model of Neutrophil Recruitment to the Lung (FIG. 6)

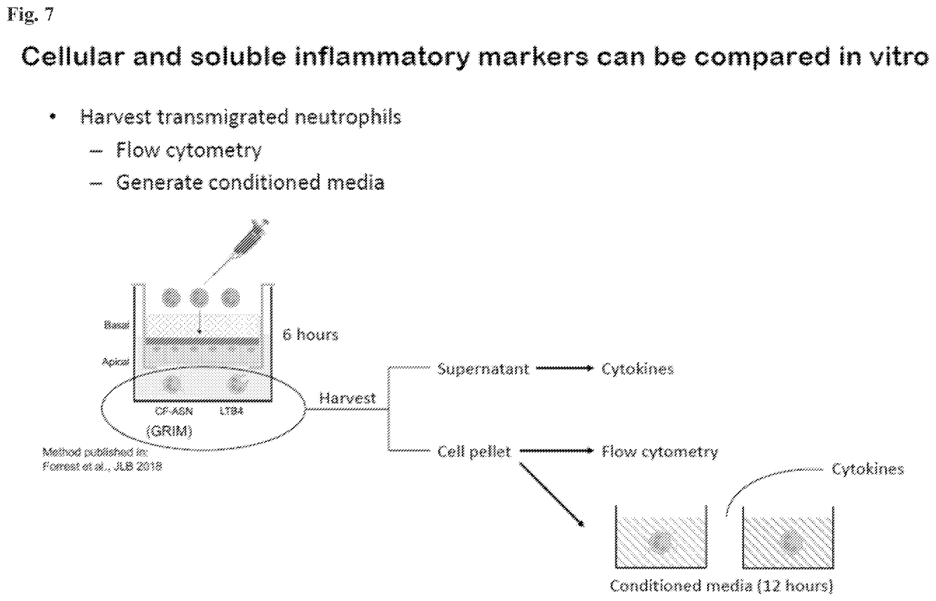

The neutrophils used are primary cells harvested from normal healthy volunteers immediately prior to the study. Cellular and soluble inflammatory markers were compared in vitro as described in FIG. 7.

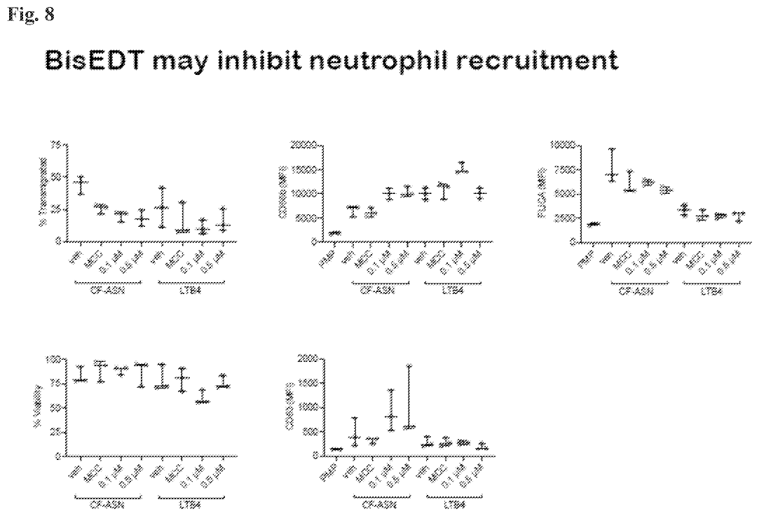

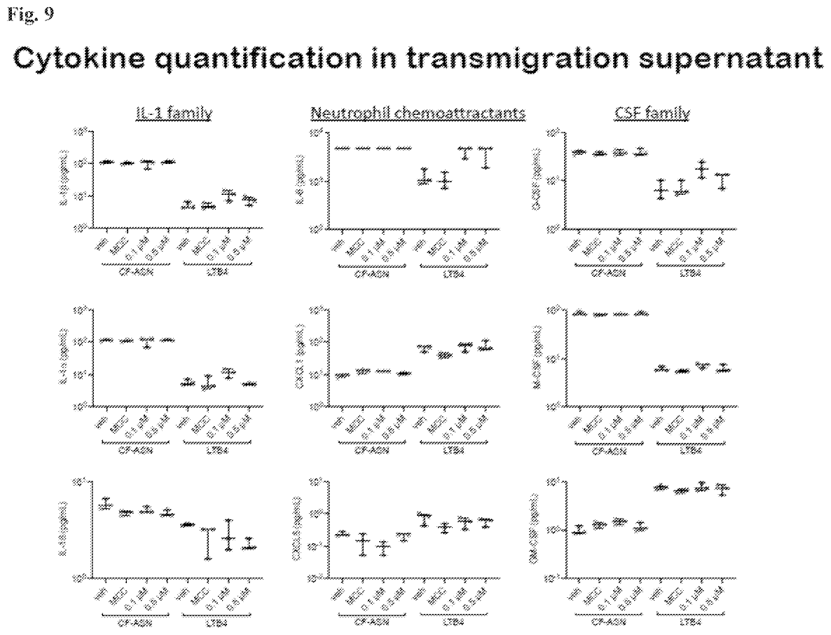

FIG. 8: The data shown in the graphs demonstrate the lower level of neutrophil transmigration (always compared to the vehicle) in both groups, as well as very high level of viability of the cells in both the CF-conditioned media experiment and ‘normal’ media (LTB4) experiment. Slowing the transmigration of the neutrophils suggests the same can happen in human subjects, which means attracting fewer inflammatory cells (less inflammation). This outcome is consistent with the increased expression of IL-10 is the graph showing FLICA (IMF), which is the measure of the activity of caspase-1, the major proteolytic enzyme that cleaves pro-IL-1β and pro-IL-18 to their active forms. According to the data, activity of caspase-1 is slightly reduced. This can be interpreted as a direct inflammasome inhibitory effect.

FIG. 9: Cytokine quantification in transmigration supernatant (assessed after the experiment had run for 6 hours) shows an appreciably lower level of IL-18 (an inflammasome cytokine) compared to vehicle.

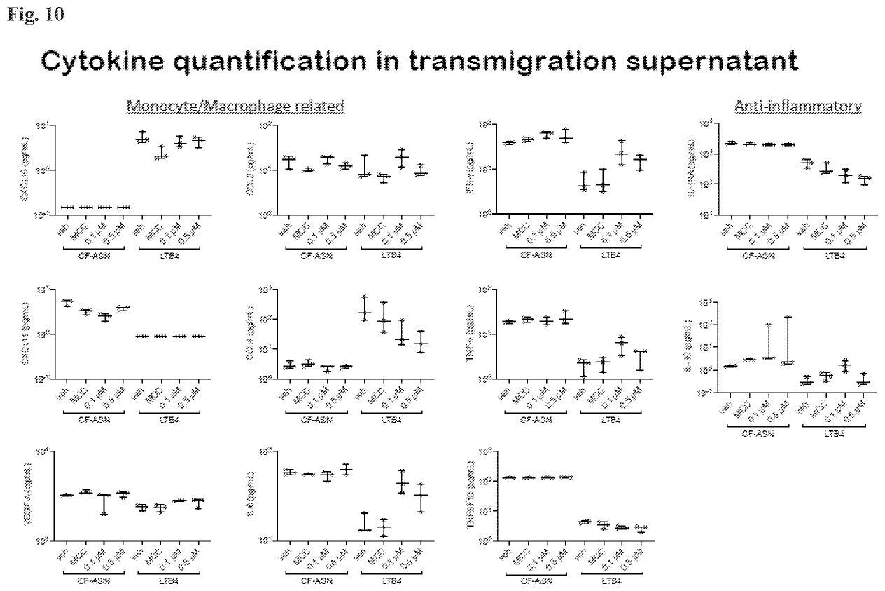

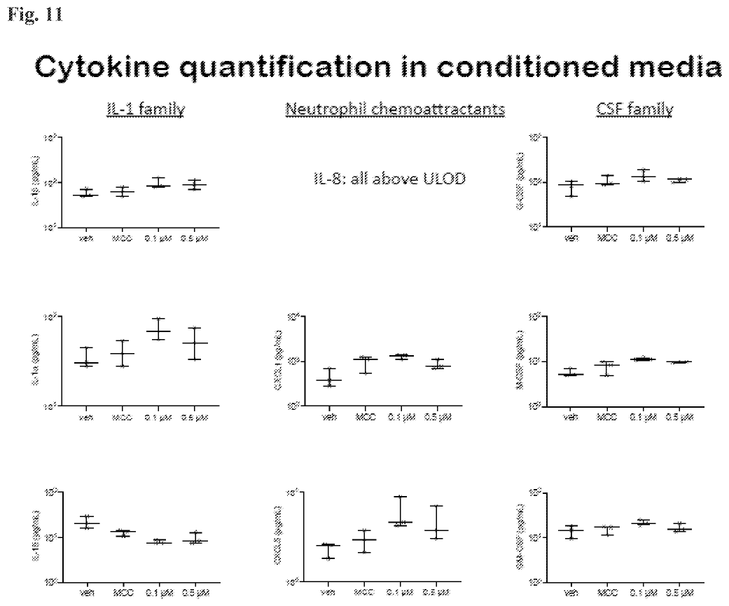

FIG. 10: At the 6-hour time point, other than the important impact on neutrophil transmigration as described above, it is notable that CCL4 (macrophage inflammatory protein), which acts as a chemoattractant for macrophages, natural killer cells, and other immune cells, is reduced with BisEDT. This would have an anti-inflammatory effect.

FIG. 11: IL-18 (a proinflammatory cytokine that is upregulated if inflammasomes are activated) is down-regulated by BisEDT, compared to vehicle.

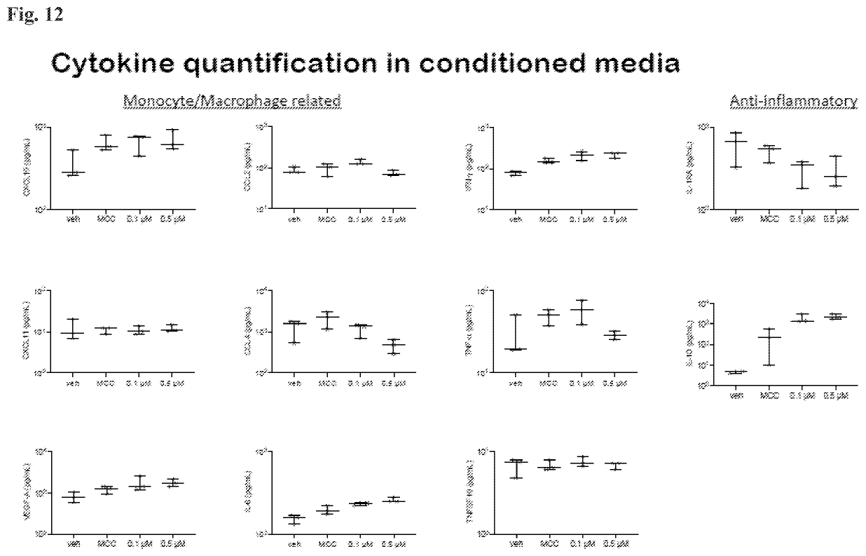

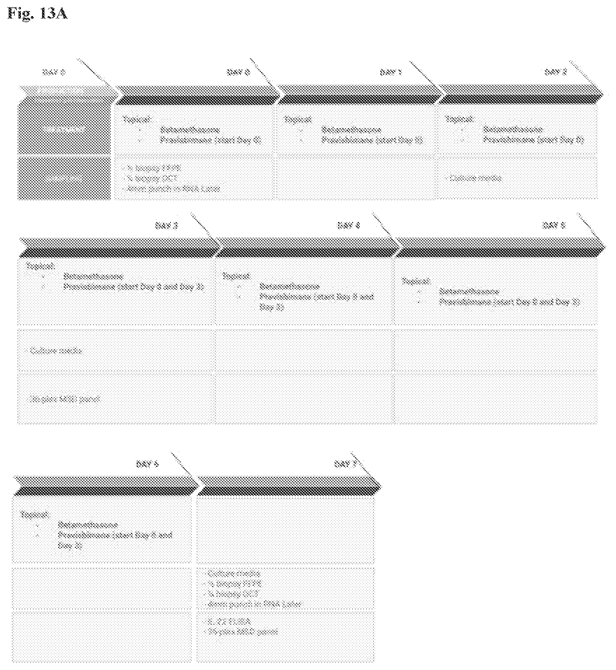

FIG. 12: The ‘conditioned media’ portion of this assay occurs after (a) the first experiment which takes 6 hours, and (b) an additional 12 hours in which the cells can be influenced by BisEDT. The cells have that length of time to secrete various factors (or not) into the conditioned media. Graphs show is the expression of IL-10 by the neutrophils at this time point. Specifically, the data pertains to the neutrophils that have been conditioned by the CF media. What is observed is a 200- to 300-fold increase in the expression of IL-10. The inhibition of neutrophil transmigration is consistent with this high level of expression of IL-10. Very minor changes are observed in other cytokines including interferon-gamma and IL-6 (both proinflammatory), but the most substantial change is related to the increased expression of IL-10, which is strongly anti-inflammatory. As described, the inhibition of caspase-1 is also consistent with increased expression of IL-10. There is also a small increase in the expression of VEGF, which is a growth factor that aids in increasing capillary beds to improve tissue perfusion, among other things.

### Summary

From these studies, reduced transmigration efficiency was observed in the presence of pravibismane, with no effect on cell viability. A slight reduction in caspase-1 activity was observed, and IL-10 secretion was substantially increased.

### Example 8: Effectiveness of BisEDT in an Ex Vivo Model for the Treatment of Psoriasis

This Example demonstrates the anti-inflammatory effects of topically applied BisEDT formulation in a human skin model for psoriasis.

Background: InflammaSkin® model is a validated psoriasis-like model developed to reproduce key features of pro-Th17/Th1 inflammation associated with psoriasis. It relies on the in situ activation of resident T-cell of normal skin biopsies and their further polarization into Th17/Th1 phenotype with the supplementation of culture medium with a cocktail of pro-inflammatory cytokines.

Table 5 provides a description of the test articles being evaluated in the model.

TABLE 5
Characteristics of Test Articles (TA)

| | |
|---|---|
| Name | BisEDT |
| | (Topical) |
| Reference | BisEDT |
| | 2.5 mg/mL |
| Number of concentrations | One |
| tested | |

Study Parameters: Skin used in the models was from female donors with no record of current inflammatory skin disease or treatment.

Model production and culture: 2 HypoSkin models of 15 mm in diameter (1.76 cm²) were produced from 1 donor and cultured with 2 mL standard HypoSkin® medium. 20 Hypo-InflammaSkin models were produced from 1 donor by inducing in sit activation and Th17/Th1 polarization of skin resident T cells with proprietary cocktail and cultured with 2 mL Hypo-InflammaSkin® culture medium. The total treatable surface area of the models was 113 mm², and dosing of BisEDT took this into account based on dosing of the topical treatment. Models were maintained in standard cell culture conditions for the whole culture duration at 37° C., 5% CO₂ and water saturation, with appropriate culture medium renewed every day.

Treatment Groups: Model Treatment: (1) HypoSkin® untreated control; (2) Hypo-InflammaSkin® untreated control; (3) topical betamethasone (positive control); (4) topical BisEDT—2.5 mg/mL (treatment applied every day from day 0 to 6; (5) topical BisEDT—1.25 mg/mL (treatment applied every day from day 3 to 6). N=3 per treatment group, except N=5 for Hypo-InflammaSkin® and N=1 for HypoSkin®.

Topical Treatment—Betamethasone (control): 25 μL of betamethasone was topically applied onto the surface of each skin model. Topical application was performed from Day 0 to Day 6 after removal of the previous treatment residues using a cotton swab.

Topical Treatment—BisEDT: 36 μL of the test compound was topically applied onto the surface of each skin model, massaging across the entire surface area for 1 minute, using a parafilm wrapped cotton swab; the compound was left on the skin. Topical application was performed from Day 0 to Day 6 (Treatment Group 4) or Day 3 to Day 6 (Treatment Group 5). A total of 9 models were topically treated.

Culture Media—culture media was collected on Day 3 prior to treatment application and on Day 7 and frozen at −80° C. A total of 42 culture media samples were collected.

Skin Biopsies—sampling of skin models was performed on Day 0 and Day 7, the hypodermis was removed, the biopsy repunched in order to eliminate the area under the silicon ring, and processed as follows:
½ biopsy fixed in 10% buffered formalin and processed for paraffin wax embedding.
¼ biopsy placed in OCT, snap frozen, and stored at −80° C.
a 4 mm biopsy punch on the last % biopsy and stored in RNA Later.

A total of 22 models were sampled.

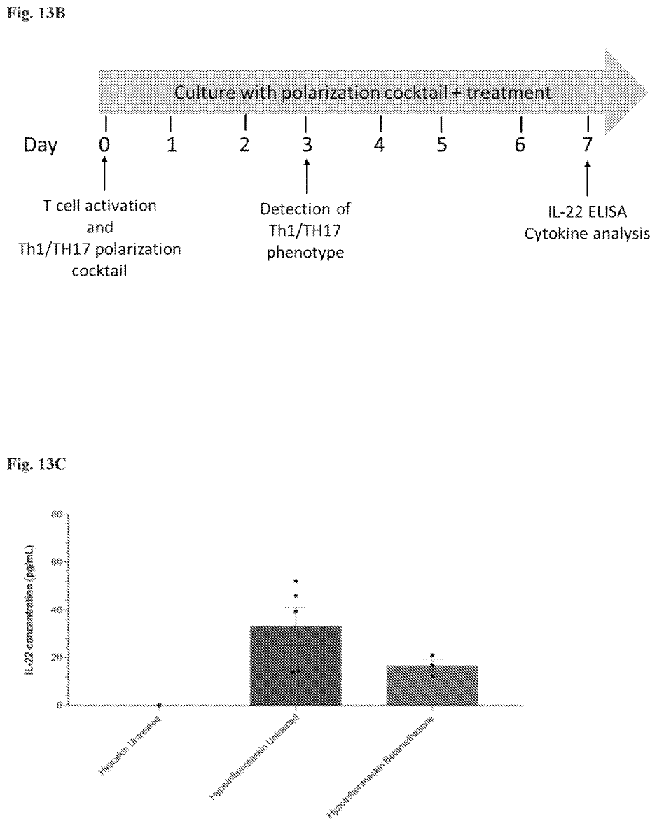

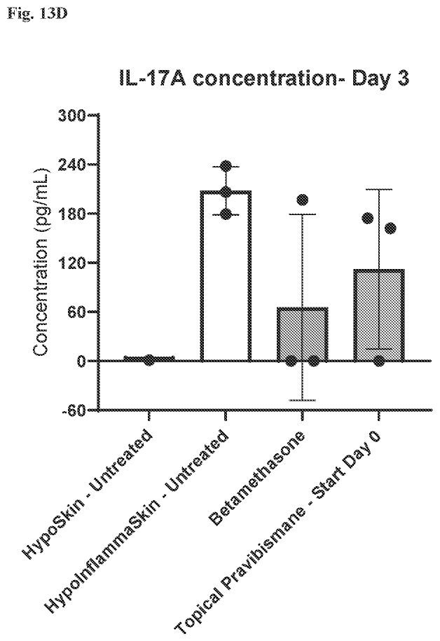

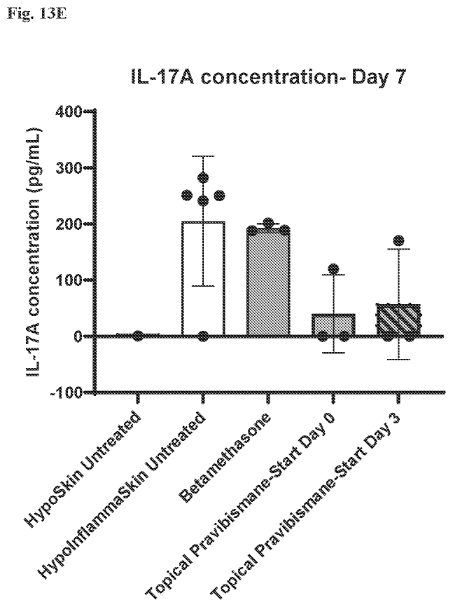

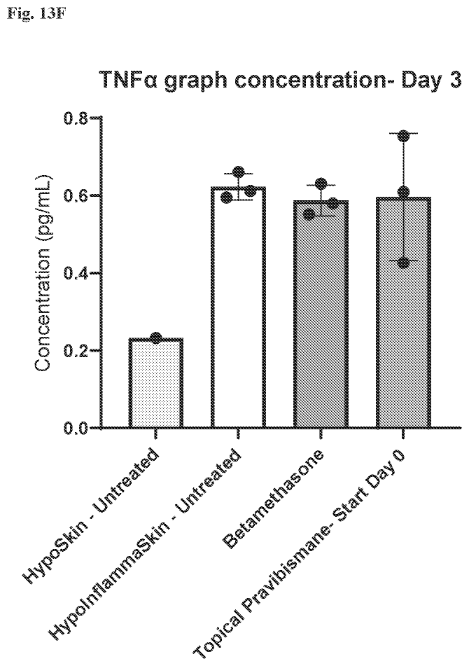

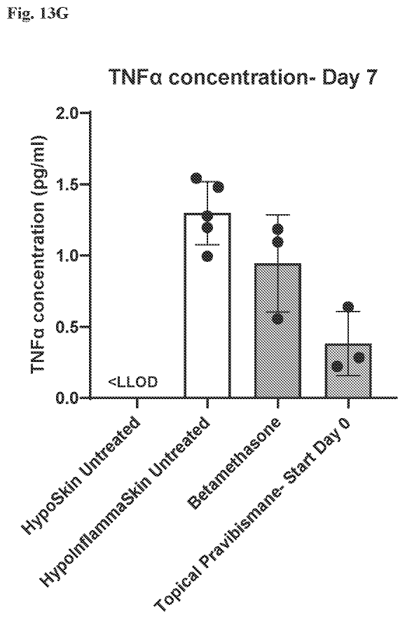

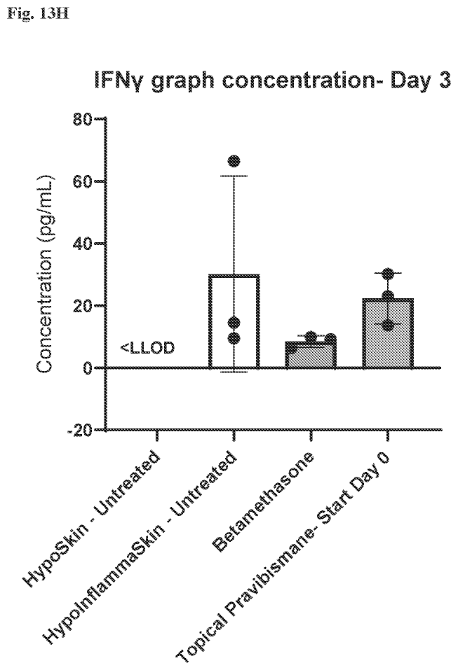

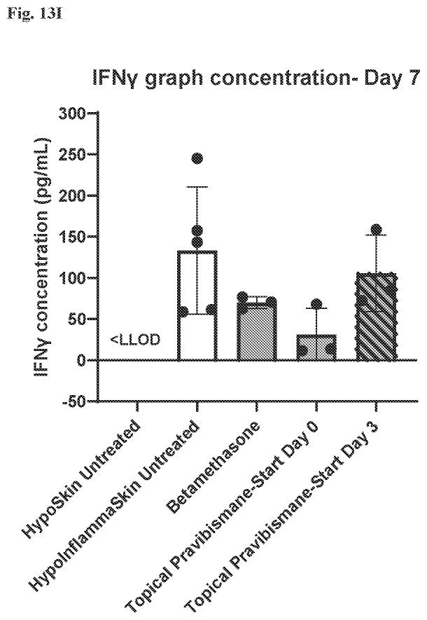

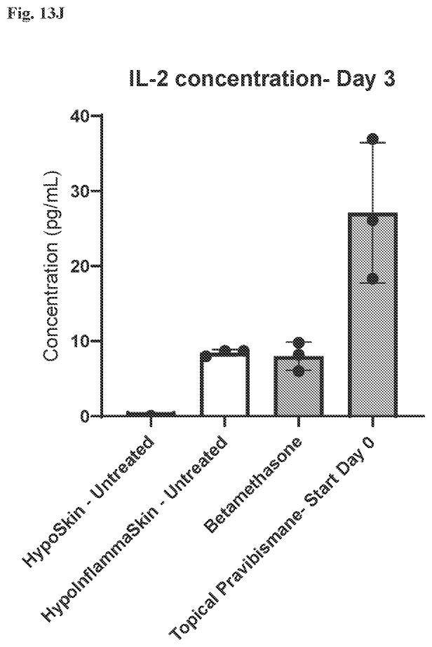

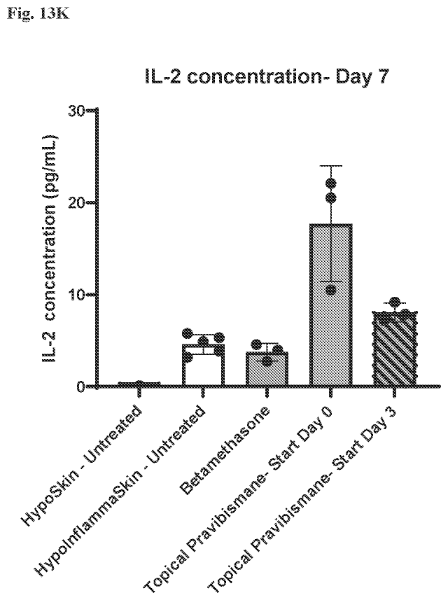

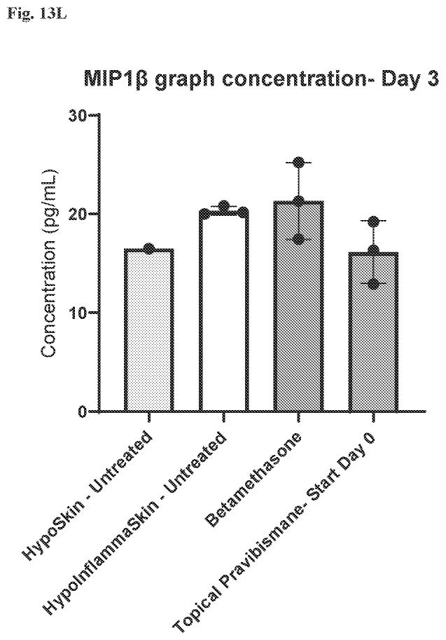

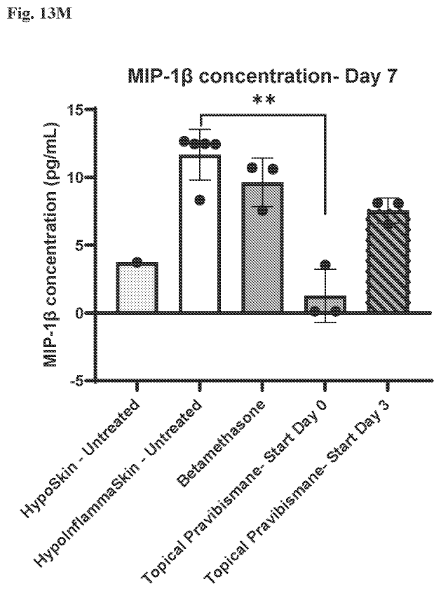

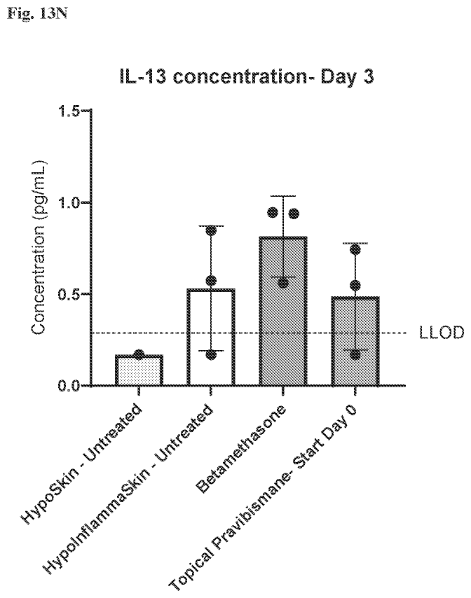

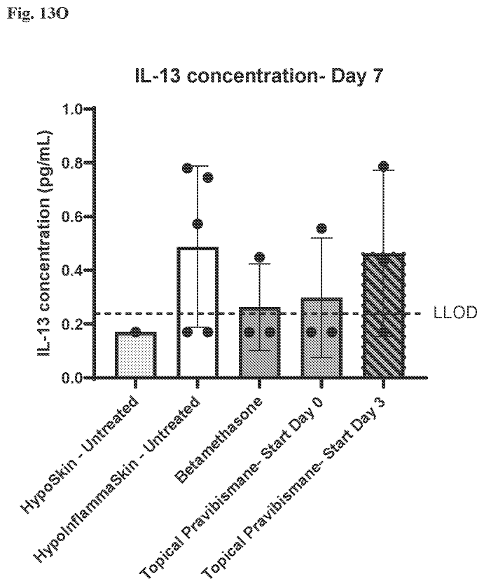

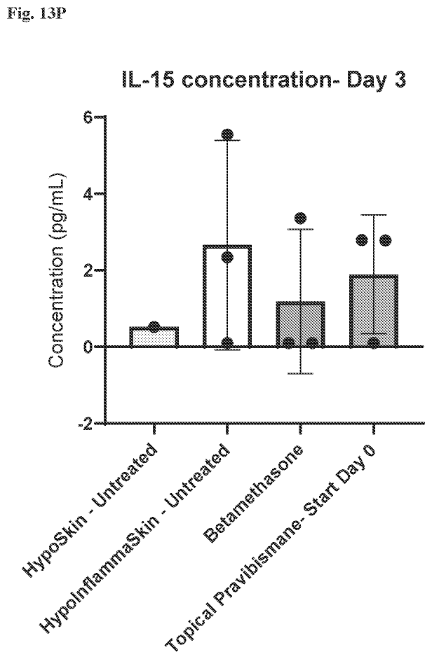

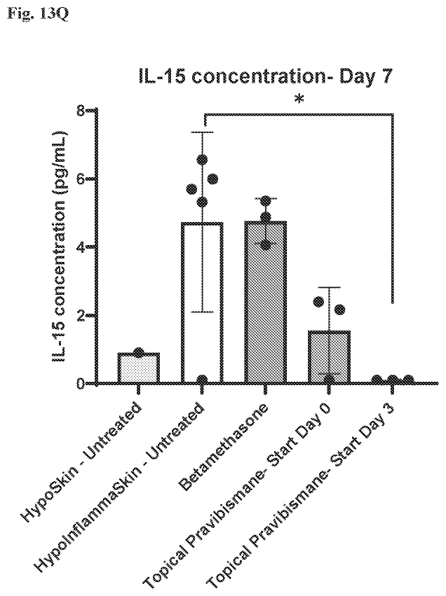

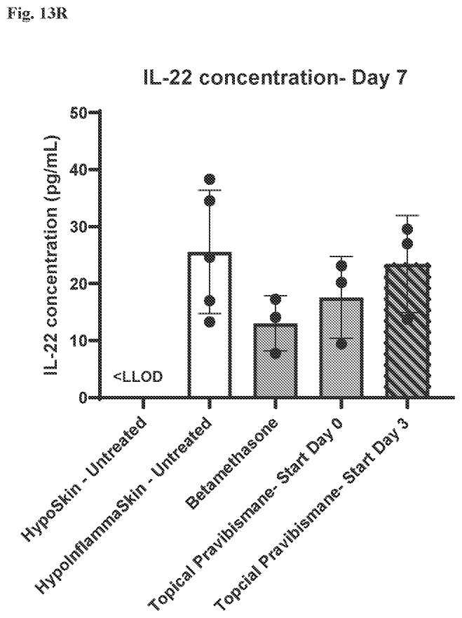

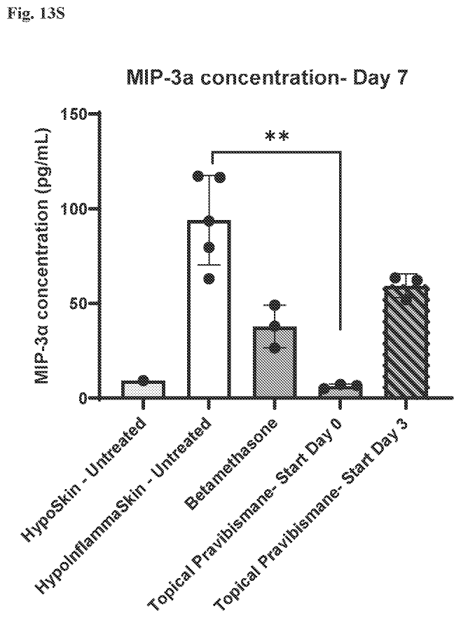

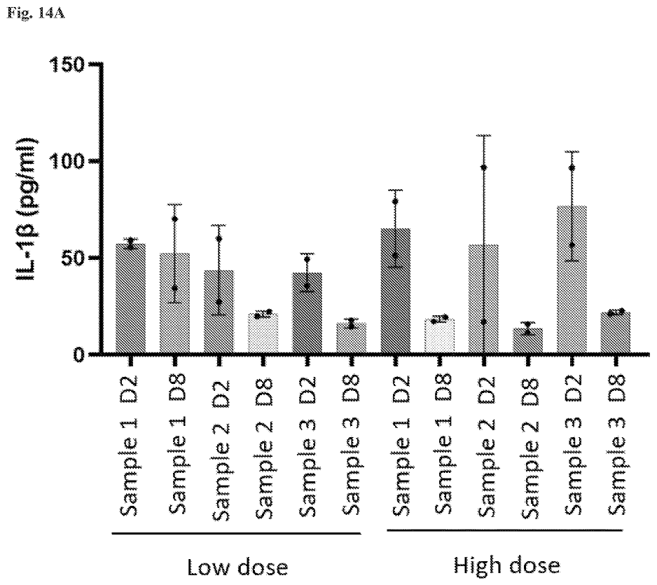

An experimental workflow is provided in FIG. 13A, FIG. 13B, and Table 6 provides the culture conditions for each donor.

TABLE 6
Culture conditions for each donor.

| | | | | | | | | Sampling/Analysis Timepoint | | |
|---|---|---|---|---|---|---|---|---|---|---|
| | | | | | | | | | Day 3 | |
| | | Treatment | | | | | | | Culture | |
| | | Compound | | | | Start | End | | medium | |
| Condition | Model | name | Dose | Route | Frequency | date | date | Day 0 | only | Day 2 |
| 1 | HypoSkin | Control | N/A | | | | | n = 1 | (n = 1) | n = 1 |
| | | Untreated | | | | | | | | |
| 2 | Hypo- | Control | N/A | | | | | — | (n = 5) | n = 5 |
| | InflammaSkin | Untreated | | | | | | | | |
| 3 | | Positive control | 0.05% | Topical | Daily | Day 0 | Day 6 | — | (n = 3) | n = 3 |
| | | Betamethasone | | 25 μL | | | | | | |
| 4 | | Pravibismane | 2.5 | Topical | Daily | Day 0 | Day 6 | — | (n = 3) | n = 3 |
| 5 | | | mg/mL | 36 μL | | Day 3 | Day 6 | — | (n = 3) | n = 3 |

| | |
|---|---|
| Total number of HypoSkin models | n = 2 |
| Total number of Hypo-InflammaSkin models | n = 20 |

### Analysis

IL-22 analysis was carried out on control conditions to confirm the donor's response to T-cell activation and to the positive treatment control.

IL-22 release: IL-22 release was measured in culture media using ELISA Human IL-22 Quantikine Kit (Bio-techne, ref D2200). Only samples from control conditions 1 to 3 were analyzed to assess donor's response to T cell activation. Each sample was analyzed in duplicate.

A total of 9 samples were analyzed on ELISA for IL-22 release.

Results: A decrease in IL-22 release was observed following treatment with betamethasone, with an average concentration of 16.67 μg/mL, suggesting that the treatment successfully reduced the inflammatory response (FIG. 13Q).

MSD Immunoassay—cytokine release (IL-2, IL-13, IL-15, IL-17A, IL-22, TNF-α, INF-γ, MIP-1β, and UIP-3α) in the culture medium was measured using the V-PLEX Plus Human Cytokine Kit (KI5089G, MesoScale Discovery) (FIGS. 13D-13S).

### Cytokine Analysis (Day 3 and Day 7):

IL-17A (FIGS. 13D and 13E): IL-17A is a key cytokine upregulated in psoriatic skin. Topical pravibismane results in notable reduction of IL-17A secretion compared to untreated HypoInflammaSkin®. Topical pravibismane (started at day 0 or day3) reduces IL-17A levels even lower than positive control (betamethasone, also started at Day 0). Greater decrease in IL-17A levels upon longer duration of pravibismane exposure.

TNF-α (FIGS. 13F and 13G): Pravibismane resulted in reduction in TNF-α compared to untreated HypoInflammaSkin® control. Pravibismane treatment starting at Day 0 reduced TNF-α levels even lower than positive control (betamethasone, also started at Day 0).

IFN-γ (FIGS. 13H and 13I): Pravibismane reduced IFNγ secretion compared to untreated HypoInflammaSkin® control. Pravibismane treatment starting at Day 0 reduced IFN-7 levels even lower than positive control (betamethasone, also started at Day 0). More profound reduction in IFN-7 secretion was achieved with longer duration of pravibismane treatment.

IL-2 (FIGS. 13J and 13K): Pravibismane treatment increased IL-2 secretion by over 3-fold compared to HypoInflammaSkin® control. IL-2 is known to inhibit TH17 mediated responses and may be the basis for downregulation of IL-17.

MIP-1β (FIGS. 13L and 13M): Pravibismane resulted in statistically significant decrease in secretion of MIP-10 compared to untreated HypoInflammaSkin®. MIP-1β levels were lower than the steroid betamethasone. Greater decrease in MIP-1β levels were achieved upon longer duration of pravibismane exposure.

IL-13 (FIGS. 13N and 13O): Pravibismane (day 0) reduced IL-13 secretion compared to HypoInflammaSkin® control.

IL-15 (FIGS. 13P and 13Q): Pravibismane (day 3) resulted in statistically significant reduction of IL-15 secretion compared to untreated HypoInflammaSkin®. IL-15 secretion in pravibismane treatment (day 3) was even lower than healthy uninflamed skin (HypoSkin® untreated).

IL-22 (FIG. 13R): Pravibismane resulted in reduced secretion of IL-22 compared to untreated HypoInflammaSkin® control. Greater decrease in IL-22 levels were achieved upon longer duration of pravibismane exposure.

MIP-3α (FIG. 13S): Pravibismane resulted in statistically significant decrease in secretion of MIP-3α compared to untreated HypoInflammaSkin®. Greater decrease in MIP-3α levels were achieved upon longer duration of pravibismane exposure.

Day 3 Summary: Compared to HypoInflammaskin®, topical pravibismane resulted in reduced secretion of IL-17A, TNF-α, INF-γ, MIP-110, IL-13, and IL-15. Pravibismane treatment increased IL-2 secretion by over 3-fold from HypoInflammaSkin controls. IL-2 is known to inhibit TH17 mediated responses and could be the basis for downregulation of IL-17 by day 7.

Day 7 Summary: Betamethasone treatment decreased the secretion of many cytokines compared to the untreated HypoInflammaSkin® controls, including IFN-γ, IL-17A, IL-22, MIP-3α, IL-13, IL-15, and TNF-α. By Day 7, topical application of pravibismane on Day 0 continued to decrease many cytokines compared to the untreated HypoInflammaSkin® controls, including cytokines such as IFN-γ, MIP-3α, MIP-1β, TNF-α, and LL-22, with IL-17A, IL-15, and IL-13. Pravibismane noticeably induced IL-2 secretion (˜3 fold) compared to HypoInflammaSkin® control. Pravibismane continued to further decrease other cytokines, even more so than the variation noted in the HypoInflammaSkin® untreated group—notable cytokines related to this observation included MIP-3α, TNF-α (3.3-fold less than that of untreated HypoInflammaSkin group), MIP-1β (two out of three replicates below LOQ).

Surprisingly, pravibismane appeared to be more effective than betamethasone at reducing/preventing inflammation induced in the HypoInflammaSkin® models.

### Example 9. Pulmonary Anti-Inflammatory Effect with Inhaled BisEDT

Two concentrations (low: 1 mg/kg; high: 7 mg/kg) of BisEDT were evaluated by inhalation in rats.

Bronchoalveolar lavage fluid (BALF) samples from day 2 (D2) and day 8 (D8) post treatment were analyzed for the expression of cytokines.

Protocol: Sprague-Dawley rats were randomly assigned to a study group based on body weight. Adult, male Sprague-Dawley rats (n=6 per group) were exposed to the aerosolized test article (TA) with a single dose on Study Day 1. Groups 1, 2, and 3 were exposed by inhalation to 1 mg/kg (targeted presented dose) of BisEDT sample 1, BisEDT sample 2, or BisEDT sample 3, respectively. Groups 4, 5, and 6 were exposed by inhalation to 7 mg/kg (targeted presented dose) of BisEDT sample 1, BisEDT sample 2, or BisEDT sample 3, respectively. Animals were necropsied on Study Days 2, 4, and 8, and terminal blood collections were conducted at that time.

Terminal blood collections were conducted on groups of 2 designated animals from each treatment group at target timepoints of 24, 72, and 168 hours following the end of the inhalation exposures. In each case, the greatest volume of blood obtainable, 2-5 ml, was collected and mixed with anti-coagulant as described below. Each terminal whole blood sample collected was divided in half, with half remaining as whole blood and half processed to plasma. Whole blood and plasma were stored frozen pending bioanalysis for total BisEDT (whole blood) or inflammatory cytokines (plasma).

Blood Processing: All blood samples were placed into a tube containing K3EDTA, and mixed thoroughly. Following mixing, blood obtained at the time of euthanasia was divided in half. Half of the blood sample was stored frozen without further processing pending bioanalysis. The remaining half was placed on wet ice before being processed to plasma by centrifugation at 2500×g for ≥10 minutes at 2 to 8° C. Plasma was aliquoted into a cryovial(s), snap frozen in liquid nitrogen, and stored at −80° C.±10° C. until cytokine analysis was performed using ELISA kits following manufacturer's instructions.

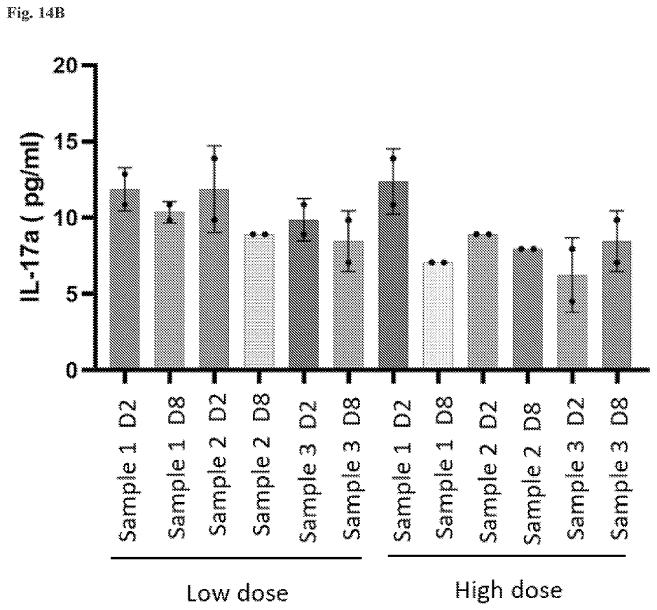

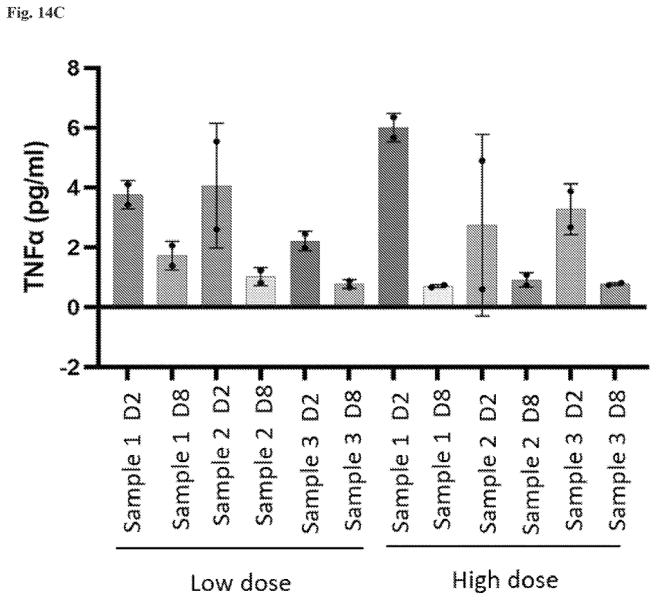

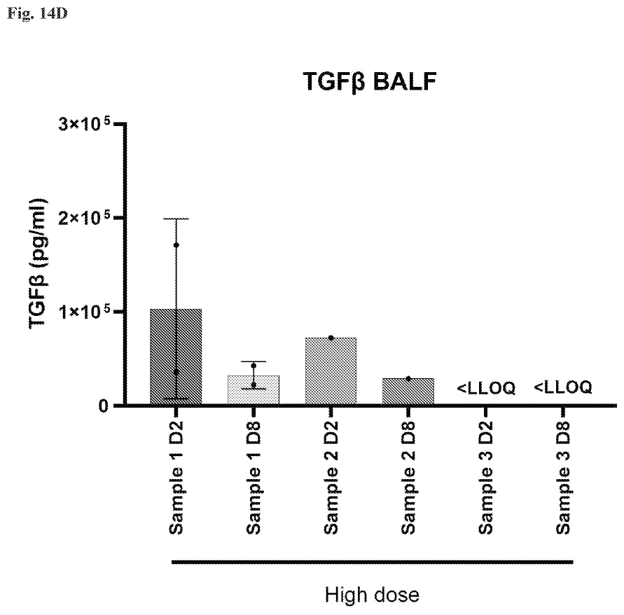

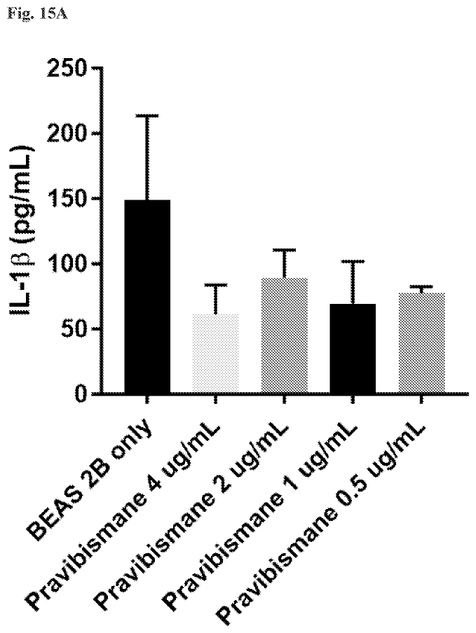

Results: Consistent reduction in the expression of pro-inflammatory cytokines IL-1β (FIG. 14A), IL-17A (FIG. 14B), and TNF-α (FIG. 14C) was observed at day 8 compared to day 2 in rats treated with a low dose or high dose of BisEDT.

A similar trend was observed for multifunctional cytokine, TGF-β (FIG. 14D).

### Example 10. Cytokine Analysis in Human Lung Epithelial Cells Treated with BisEDT

Protocol: BEAS-2B cells were grown in tissue culture flasks and a 24-well plate was seeded with 2×10⁵ cells/well and incubated at 37° C. and 5% CO₂ for 48 hours. Select wells were treated with either 4 μg/mL, 2 μg/mL, 1 μg/mL, or 0.5 μg/mL BisEDT or left untreated as a control and incubated at 37° C. and 5% CO₂ for 24 hours. Cell culture supernatant was collected from each well and stored at −20° C. until further ELISA analysis. On the day of the ELISA assays to determine the expression of each cytokine, cell culture supernatant was thawed, and the assays performed following the manufacturer's instructions. Cells were collected for RNA isolation using Qiagen RNeasy kit using manufacturers' instructions. Briefly, genomic DNA was degraded with DNase (Roche) and samples were incubated at 37° C. for 1 hour followed by 3 μL DNase inactivation reagent (Invitrogen) incubated at 37° C. for 2 minutes. Samples were centrifuged at maximum speed for 5 minutes to remove the inactivation reagent. RNA concentration and quality was evaluated using a Nanodrop. RNA samples were utilized to generate cDNA using cDNA synthesis kit (Bio-Rad). qPCR for AIM2 and actin (housekeeping gene) was conducted using SYBR green kit (Bio-Rad) along with standards.

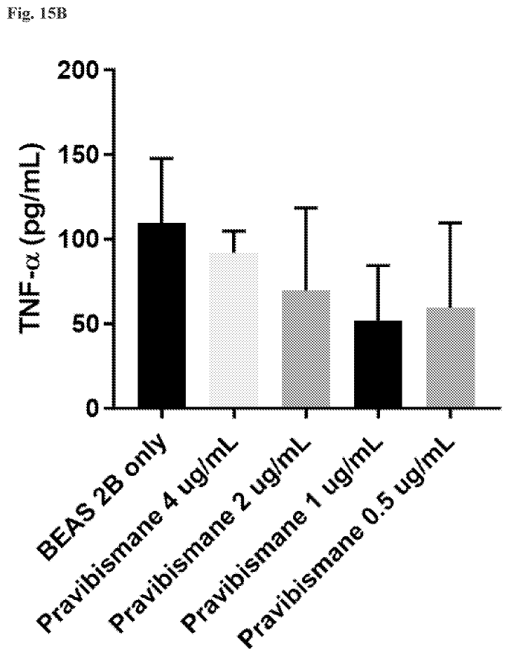

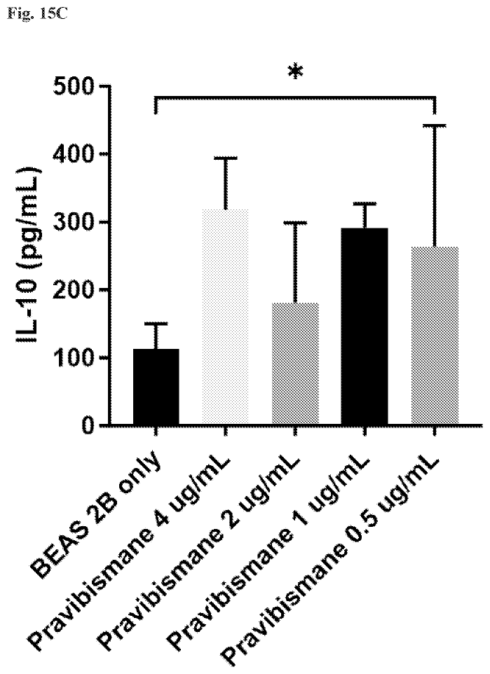

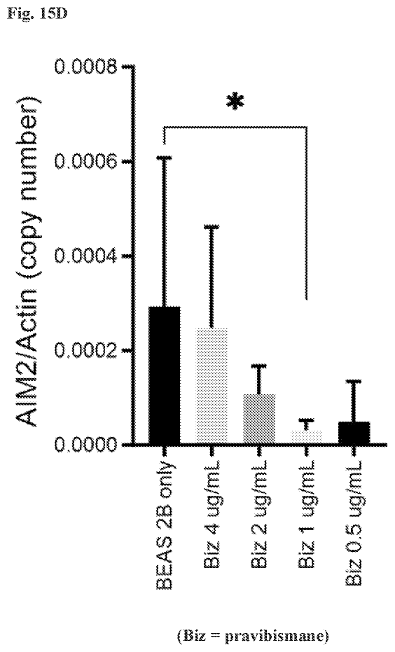

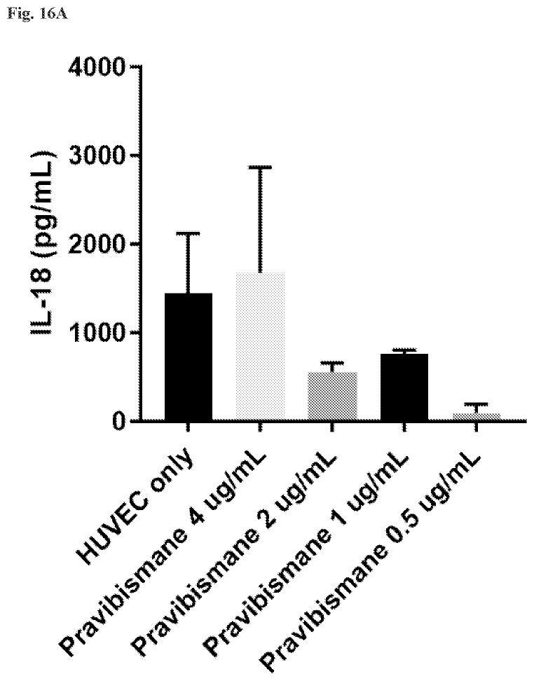

Results: Consistent reduction in the expression of pro-inflammatory cytokines IL-1β (FIG. 15A) and TNF-α (FIG. 15B) was observed in BEAS-2B cell treated with 0.5 μg/mL, 1 μg/mL, 2 μg/mL, or 4 μg/mL of BisEDT (pravibismane) compared to untreated cells.

A consistent increase in the expression of anti-inflammatory cytokine IL-10 (FIG. 15C) was observed BEAS-2B cell treated with 0.5 μg/mL, 1 μg/mL, 2 μg/mL, or 4 μg/mL of BisEDT (pravibismane) compared to untreated cells.

Like in THP-1 cells, BisEDT was also shown to inhibit AIM2 inflammasome expression in BEAS-2B cells (FIG. 15D).

### Example 11. Cytokine Analysis in Human Umbilical Vein Endothelial Cells Treated with BisEDT

Protocol: HUVEC cells were grown in tissue culture flasks and a 24-well plate was seeded with 2×10⁵ cells/well and incubated at 37° C. and 5% CO₂ for 48 hours. Select wells were treated with either 4 μg/mL, 2 μg/mL, 1 μg/mL, or 0.5 μg/mL BisEDT or left untreated as a control and incubated at 37° C. and 5% CO₂ for 24 hours. Cell culture supernatant was collected from each well and stored at −20° C. until further ELISA analysis. On the day of the ELISA assays to determine the expression of each cytokine, cell culture supernatant was thawed, and the assays performed following the manufacturer's instructions. Cells were collected for RNA isolation using Qiagen RNeasy kit using manufacturer's instructions. Briefly, genomic DNA was degraded with DNase (Roche) and samples were incubated at 37° C. for 1 hour followed by 3 μL DNase inactivation reagent (Invitrogen) incubated at 37° C. for 2 minutes. Samples were centrifuged at maximum speed for 5 minutes to remove the inactivation reagent. RNA concentration and quality was evaluated using a Nanodrop. RNA samples were utilized to generate cDNA using cDNA synthesis kit (Bio-Rad). qPCR for AIM2 and actin (housekeeping gene) was conducted using SYBR green kit (Bio-Rad) along with standard.

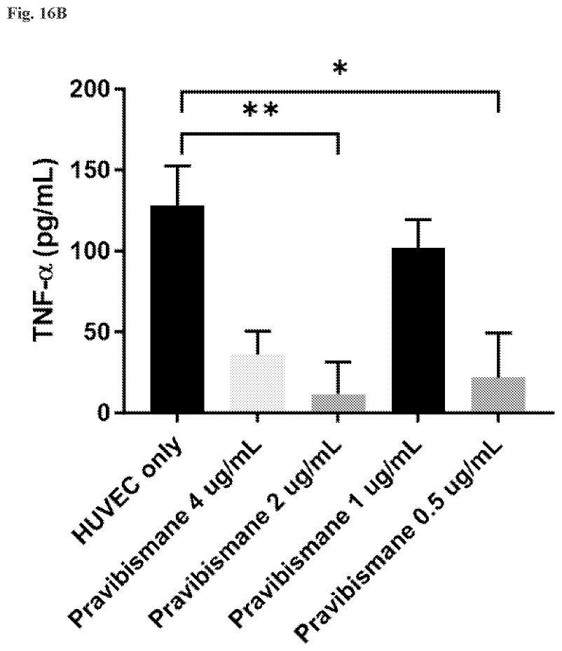

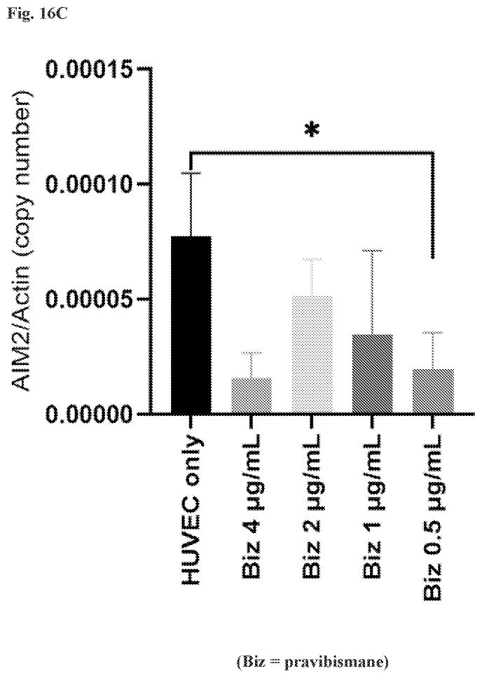

Results: Consistent reduction in the expression of pro-inflammatory cytokines IL-18 (FIG. 16A) and TNF-α (FIG. 16B) was observed in HUVEC cell treated with 0.5 μg/mL, 1 μg/mL, 2 μg/mL, or 4 μg/mL of BisEDT compared to untreated cells.

BisEDT was also shown to inhibit AIM2 inflammasome expression (FIG. 16C) in HUVEC cell treated with 0.5 μg/mL, 1 μg/mL, 2 μg/mL, or 4 μg/mL of BisEDT compared to untreated cells.

### Example 12. Cytokine Analysis in Human Alveolar Cells Treated with BisEDT

Protocol: A549 cells were grown in tissue culture flasks and a 24-well plate was seeded with 2×10⁵ cells/well and incubated at 37° C. and 5% CO₂ for 48 hours. Select wells were treated with either 4 μg/mL, 2 μg/mL, 1 μg/mL, or 0.5 μg/mL BisEDT or left untreated as a control and incubated at 37° C. and 5% CO₂ for 24 hours. Cell culture supernatant was collected from each well and stored at −20° C. until further analysis. Cells were collected for RNA isolation using Qiagen RNeasy kit using manufacturer's instructions. Briefly, genomic DNA was degraded with DNase (Roche) and samples were incubated at 37° C. for 1 hour followed by 3 μL DNase inactivation reagent (Invitrogen) incubated at 37° C. for 2 minutes. Samples were centrifuged at maximum speed for 5 minutes to remove the inactivation reagent. RNA concentration and quality was evaluated using a Nanodrop. RNA samples were utilized to generate cDNA using cDNA synthesis kit (Bio-Rad). qPCR for NLRP3 and actin (housekeeping gene) was conducted using SYBR green kit (Bio-Rad) along with standard.

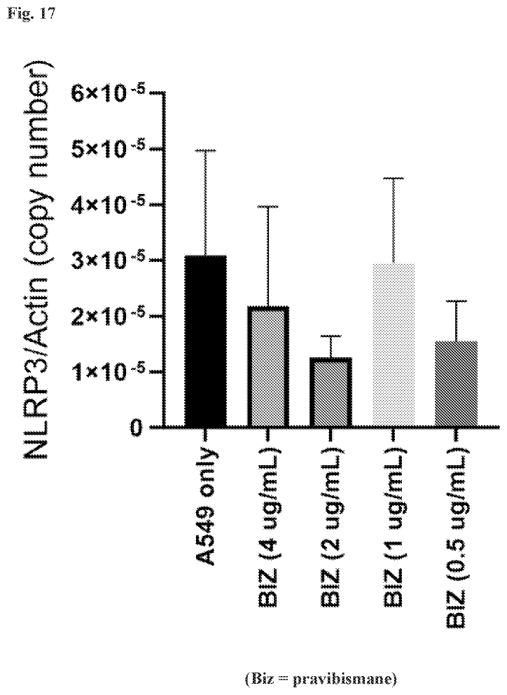

Results: NLRP3 is an inflammasome that plays a role in the release of pro-inflammatory cytokines. The data in FIG. 17 shows that BisEDT (pravibismane) inhibits NLRP3 expression in A549 cells compared to untreated cells.

### Example 13. Demonstration of Dose-Dependent Anti-Inflammatory Effect of BisEDT on FITC-Induced Contract Hypersensitivity in Mice

Protocol: Male BALB/c mice at 8 weeks old are used in the study.

All animals, except those in Group 1 (Sham), were sensitized topically (TOP) on the shaved abdominal skin with 200 μL of 0.5% Fluorescein Isothiocyanate (FITC) dissolved in 20% tributyrin in acetone on Days 1 and 2. On Day 7, animals were challenged by applying 20 μL of 0.5% FITC solution onto both sides of the right ear. Animals in Group 1 received nothing.

Vehicle, BisEDT test articles and reference compound (tacrolimus) were administered topically (TOP) once daily (QD) from Day 6 to Day 9 for 4 consecutive days (1 hour before FITC challenge on Day 7). The vehicle for BisEDT was phosphate-buffered saline (PBS).

### Test Articles:

2.5 mg/mL BisEDT topical suspension
1.25 mg/mL BisEDT topical suspension—prepared as a 1:1 dilution of 2.5 mg/mL BisEDT topical suspension in sterile water and vortexed to ensure homogeneity

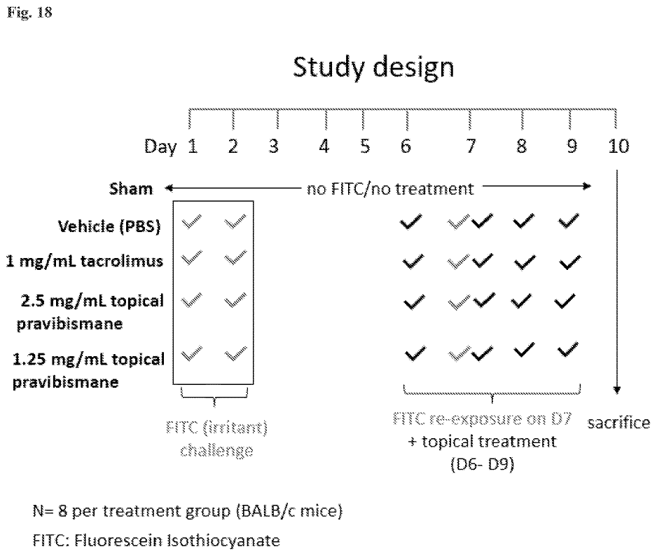

The study was conducted according to parameters provided in Table 7 and FIG. 18.

TABLE 7
Study Design

| | Antigen | Test | | Conc. | Dose | | Mice |
|---|---|---|---|---|---|---|---|
| Group | challenge | Article | Route | mg/mL | uL/mouse | mg/mouse | Male |
| 1 | None | — (Sham) | — | — | — | — | 8 |
| 2 | FITC | Vehicle (PBS) | TOP | — | 20 | QD × 4 | 8 |
| 3 | FITC | Tacrolimus | TOP | 1 | 20 | 0.02, QD × 4 | 8 |
| 4 | FITC | BisEDT | TOP | 2.5 | 20 | 0.05, QD × 4 | 8 |
| | | topical | | | | | |
| | | suspension | | | | | |
| 5 | FITC | BisEDT | TOP | 1.25 | 20 | 0.025, QD × 4 | 8 |
| | | topical | | | | | |
| | | suspension | | | | | |

Analysis: Ear swelling was measured by an experimenter blinded to treatment group, with a dial thickness micrometer gauge as an index of inflammation 30 min before dosing on Days 7, 8, 9 and 10. On Day 10, ear swelling was measured at 24 hours after the last dosing. Ear edema was calculated by subtracting the thickness of Day 7 (normal control) from Day n (treated ear). Percentage inhibition was calculated according to the formula: (Ic−It)/Ic×100, where Ic and It refer to the increase of ear thickness (mm) both in the control and treated animals. Body weight was recorded daily during the study period.

The animals were sacrificed on Day 10 and the ears were then harvested and weighed. Half of the right ear was fixed in 10% formalin for histopathology, and the other half was processed for cytokine measurement (IL-18, IL-6, and IL-10) using ThermoFisher Mouse ProcartaPlex Kit by Luminex.

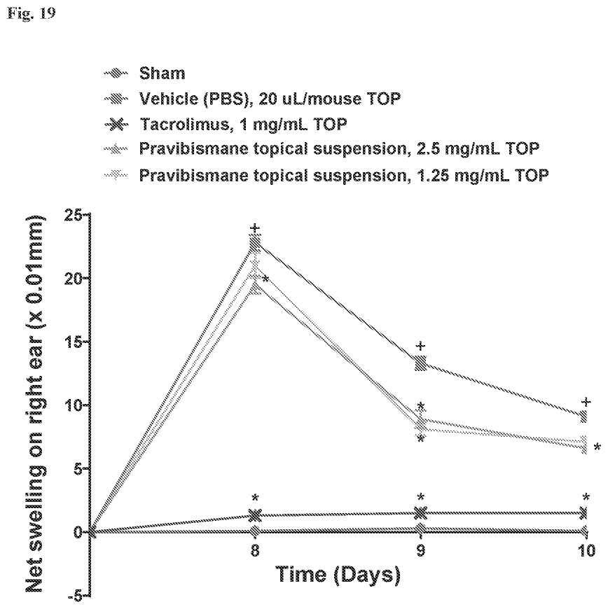

Results; Topical pravibismane treatment resulted in statistically significant and dose-dependent reduction in ear swelling, an indicator of inflammation, compared to vehicle control (FIG. 19).

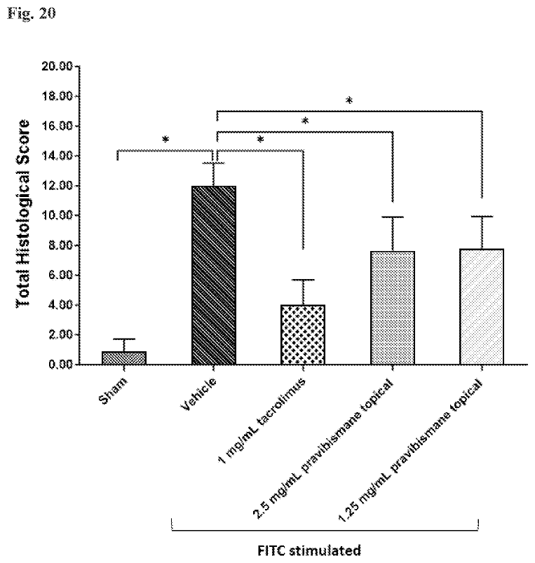

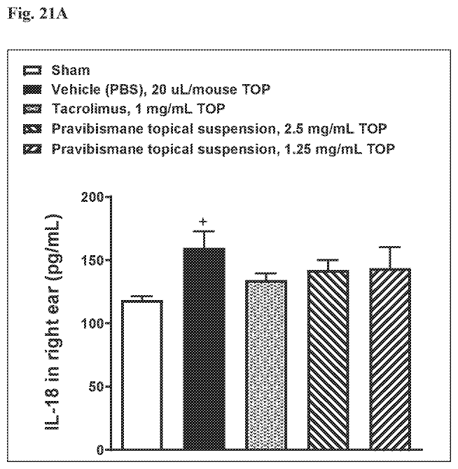

Topical pravibismane treatment also resulted in a significant reduction in histopathological score and epidermal thickness. Topical pravibismane treatment alleviates skin lesions (inflammation, necrosis, epidermal hyperplasia, and hyperkeratosis) compared to vehicle control (Table 8 and FIG. 20).

TABLE 8
Histopathological evaluation of FITC-associated skin lesions.

| | Group | | |
|---|---|---|---|
| Measurements | 1 | 2 | 3 |
| | Sham | Vehicle | Tacrolimus |
| | | | (1 mg/mL) |
| Inflammation | 0.13 ± 0.35 | 4.63 ± 0.52 ^{$} | 1.38 ± 0.52 ^{#} |
| Necrosis | 0.00 ± 0.00 | 2.00 ± 0.53 ^{$} | 0.13 ± 0.35 ^{#} |
| Hyperplasia | 0.25 ± 0.46 | 3.38 ± 0.74 ^{$} | 1.63 ± 0.52 ^{#} |
| Hyperkeratosis | 0.50 ± 0.53 | 2.00 ± 0.53 ^{$} | 0.88 ± 0.64 ^{#} |
| Total histological score | 0.88 ± 0.83 | 12.00 ± 1.51 ^{$} | 4.00 ± 1.69 ^{#} |
| Measurements [symbol] | 4 | 5 | |
| | Pravibismane topical | Pravibismane topical | |
| | suspension | suspension | |
| | (2.5 mg/mL) | (1.25 mg/mL) | |
| Inflammation | 2.38 ± 0.52 [symbol] | 2.38 ± 0.74 [symbol] | |
| Necrosis | 1.13 ± 0.83 [symbol] | 1.25 ± 0.71 [symbol] | |
| Hyperplasia | 2.75 ± 0.46 | 2.63 ± 0.74 | |
| Hyperkeratosis | 1.38 ± 0.92 | 1.50 ± 0.53 | |
| Total histological score | 7.63 ± 2.26 [symbol] | 7.75 ± 2.19 [symbol] | |

The lesion scores represent as mean ± standard deviation.
^{$} p < 0.05, Sham (Group 1) vs. Vehicle (Group 2); unpaired Student's t-test.
^{#} p < 0.05, Tacrolimus (Group 3) vs. Vehicle (Group 2); unpaired Student's t-test.
* p < 0.05, Vehicle (Group 2) vs. treated groups (Groups 4-5); one-way ANOVA followed by Dunnett's test. [symbol]
indicates data missing or illegible when filed

### Cytokine Measurement:

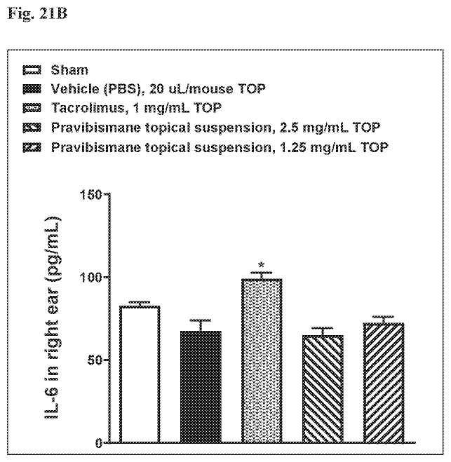

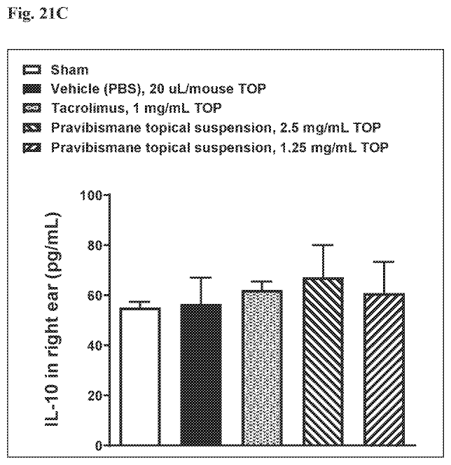

Expression of pro-inflammatory cytokine, IL-18, in the harvested right ears treated with either BisEDT topical suspension was reduced compared to vehicle control (FIG. 21A).

As shown in FIG. 21B, IL-6 expression levels were lower in harvested right ears treated with either BisEDT topical suspension compared to tacrolimus, and slightly lower than vehicle at the 2.5 mg/mL dose. Increased expression of IL-10 was observed for the 2.5 mg/mL BisEDT suspension compared to tacrolimus and vehicle control (FIG. 21C).

Results from the FITC-induced contact hypersensitivity study in mice suggest a decrease in ear swelling, decrease in the expression of pro-inflammatory cytokines and increase in expression of anti-inflammatory cytokine suggesting that pravibismane exhibits an overall anti-inflammatory effect in this model.

### Example 14: Determination of Anti-Inflammatory Potential of BisEDT in THP-1 Cells Stimulated by P. acnes

The anti-inflammatory activity of BisEDT in THP-1 cells stimulated with P. acnes (e,g, m ATCC 691) is studied.

### Method: Preparation of Stimulant for Inflammation

P. acnes culture suspension is prepared in PBS and the cell number in the suspension is adjusted to approximately 5-10⁸ CFU/ml by measuring the cell density using a Densimat. The bacterial suspension is then heat killed at 80° C. for 30 min and stored at −80° C.

### ELISA to Study Inflammatory Response in THP-1 Cells:

Cells are seeded in a 96-well format (2×10⁵ THP-1 cells per well) in media containing 10% FBS. The cells are stimulated to induce inflammatory cytokines using 3 McFarland equivalent heat-killed P. acnes. Cells in control wells are treated with PBS. One hour after induction with P. acnes, test agents (BisEDT, control(s), vehicles, etc.) are added to the induced cells at appropriate concentrations to be tested. The plates are incubated at 37° C. for 24 hours. After 24 h, the plates are centrifuged to pellet the cells and the supernatants are collected. The cell culture supernatants obtained are analyzed for levels of cytokines (e.g., IL-1α, IL-1β, IL-6, IL-8, and IL-10) by ELISA using e.g., R&D Systems kits for individual cytokines following the manufacturer's instructions.

Anti-Inflammatory Action is evaluated by a reduction in the expression of pro-inflammatory and anti-inflammatory cytokines in P. acnes-induced THP-1 cells.

THP-1 cells, induced using heat killed P. acnes, are treated with BisEDT and the levels of cytokines (e.g., IL-1α, IL-1β, IL-6, IL-8, and IL-10) are analyzed in the culture supernatant. Dexamethasone, a known anti-inflammatory agent, can be used as a positive control. The viability of cells treated with BisEDT will also be determined.

### Example 15: Evaluation of Anti-Arthritic Activity of BisEDT in mAB/LPS-Induced Experimental Murine Arthritogenic Model

The anti-arthritic activity of BisEDT administered in various amounts is assessed in a mAB/LPS-induced experimental murine arthritogenic model. The mice are administered BisEDT once daily by oral (PO) gavage throughout five successive treatment days. The treatment groups comprise n=2-8 BALB/c male mice per group. Two equally sized groups are treated with either dexamethasone (1 mg/kg; positive control) or a suspension of vehicle (e.g., 0.5% CMC/0.25% polysorbate 80), respectively.

Experimental arthritis is initially induced on Day 0 of the study by intravenous (IV) injection of a 4 monoclonal antibodies (mAB) cocktail at a dose of 100 mg/kg, followed about 72 hours later by the intraperitoneal (IP) injection of LPS 2.5 mg/kg.

Paw thickness is determined with an electronic digital caliper on six occasions (Day 0, 4, 5, 6, 7 & 9) and presented as mean group values of the average for both left and right hind paws.

Histopathological Evaluation- to determine the potential anti-arthritic activity of BisEDT, a total 4-16 animals are tested with Vehicle Control or BisEDT for histopathological evaluation. Left hind limbs are fixed in 100% neutral buffered formalin for 1 week, then transferred into buffered inorganic acid (decalcification process for about 48 hours) and back into 10% formalin prior to storage. Each limb from the middle of the tibia and distally, to include the ankle joint, i.e., the joint between the leg and foot (tibial-tarsus joint), is trimmed mid-longitudinally, and both halves are embedded in paraffin and slides of 6 micron thickness are cut and stained by hematoxylin and eosin. Histopathological changes in the joints are described and scored, using semiquantitative grading of five grades (0-4), taking into consideration the severity of the changes (0=unremarkable, 1=minimal, 2=mild, 3=moderate, 4=marked). Individual findings will be presented.

### Inhibitory Effect on the Production of Inflammatory Cytokine

ELISA is performed with the serum and foot to investigate whether or not BisEDT can suppress the generation of TNF-α and interleukin-6 (IL-6), the inflammatory cytokines known as the promoter of inflammation which is the common symptom of acute and chronic arthritis.

From the results of these studies, the therapeutic effect of BisEDT on rheumatoid arthritis is determined.

### Example 16. Evaluation of the Therapeutic Effects of BisEDT on Asthma

To investigate the therapeutic effect of BisEDT on asthma, the following experiment is performed.

### Construction of Bronchial Asthma Induced Animal Model and Treatment of Compound

6 week old Balb/c female mice in the average weight of 20 g are adapted for 1 week. 2 mg of aluminum hydroxide and 20 μg of ovalbumin are suspended in phosphate buffer (pH 7.4). Each mouse is administered with 200 μl of the phosphate buffer suspension via intraperitoneal injection for sensitization at 2-week intervals. Mice inhale 1% ovalbumin (OVA) through an ultrasonic atomizer for 30 minutes a day for 3 days after the first intraperitoneal injection of ovalbumin. 24 hours after the antigen administration, airway hyperreactivity is measured. 48 hours later, a lethal dose of pentobarbital is administered to the mouse. The bronchus is opened and the alveoli are washed with 1.4 ml of saline. Then, the test samples are obtained. The normal control (NC) is not administered ovalbumin nor inhaled. The asthma induced group (OVA) is administered with ovalbumin and inhaled to induce bronchial asthma. The comparative control is orally administered dexamethasone (2 mg/kg, PO) one hour before the inhalation (DEX). The experimental group is orally administered BisEDT. Each group is composed of 2-7 Balb/c mice.

### Airway Hyperreactivity Induced by Asthma

Airway resistance is measured by using one chamber plethysmography to obtain Penh (enhanced pause) values which are the mathematically calculated numbers reflecting airway obstruction in order to measure airway hyperreactivity. To measure Penh value, basal value in normal breathing status is first measured. Then, the mouse is allowed to intake PBS by using ultrasonic atomizer for 3 minutes. The measurement is continued for 3 minutes. Methacholine, the histamine used for the general diagnosis of bronchial asthma, is treated to the mouse with increasing the concentration from 5 mg/ml and then 10 mg/ml and to 20 mg/ml, followed by measuring Penh values.

### Investigation of Cytokine Production in Bronchoalveolar Lavage Fluid

Th2 cytokine (interleukin 5, 13) in bronchoalveolar is measured by sandwich type enzyme immunoassay. Bronchoalveolar lavage fluid obtained from each experimental group is distributed in a 96-well plate containing cytokine antibody, followed by antigen-antibody reaction at room temperature for 2 hours. To measure the content of interleukin 5 and 13, cytokine specific ELISA kit is used. The content of each cytokine is measured using the kit according to the manufacturer's instruction.

### Level of Serum IgE

The level of serum IgE relating to the severity of asthma is measured by enzyme immunoassay. A 96-well plate (ELISA plate) is coated with 20 μg/ml of ovalbumin (OVA) dissolved in 0.1 M NaHCO₃ buffer (pH 8.3) for overnight at 4° C. Serum taken from each group is distributed in the 96-well plate coated with ovalbumin. Non-specific reaction is blocked by PBS containing 1% bovine serum albumin. The serum sample is diluted at the ratio of 1:400, followed by reaction at room temperature for 2 hours. After washing well, the anti-mouse IgE monoclonal antibody is diluted at the ratio of 1:300, followed by reaction for hours. The plate is reacted with the HRP-conjugated goat anti-rat IgG polyclonal A (1:4000) at room temperature for 1 hour, followed by washing. Color development was induced with 3,3′,5,5′-tetramethylbezidine. Then, OD₆₅₀ is measured.

### Number of Inflammatory Cells in Bronchoalveolar Lavage Fluid

The number of inflammatory cells in bronchoalveolar lavage fluid is measured by Diff-Quick staining.

### Histopathological Analysis

The lung is extracted from the mouse without bronchoalveolar lavage. After formalin fixation and paraffin embedding, 4 μm thick permanent tissue sections is prepared, followed by hematoxylin & eosin (H&E) staining.

To evaluate the level of mucus production by investigating the increase of the number of goblet cells, the mucus secretion cells, the lung is extracted from the mouse without bronchoalveolar lavage. After formalin fixation and paraffin embedding, 4 μm thick permanent tissue sections are prepared, followed by Periodic acid Schiff (PAS) staining. The proliferation of goblet cells is evaluated by measuring the ratio of the goblet cells selected by the staining above in bronchial epithelial cells.

From the results of these studies, the therapeutic effect of BisEDT on asthma is determined.

### Example 17: DNFB Mouse Model to Evaluate the Therapeutic Effects of BisEDT in Atopic Dermatitis

### DNFB Mouse Model

BisEDT is tested in the dinitrofluorobenzene (DNFB) mouse model. DNFB is a small chemical hapten that induces a delayed-type hypersensitivity reaction with similarity to human atopic dermatitis.

Healthy female CDl mice (Crl:CDl (ICR)) are shaved on the abdomen and the nape of the neck under a 3 to 5% isoflurane anesthesia.

After body weight measurement, animals are allocated to study groups. The study is divided in two arms, separated by 1 week, in order to accommodate for the in life monitoring of scratching monitoring. Half of the animals from each group are allocated to arm 1 and arm 2, respectively.

For each topical BisEDT treatment under evaluation, the formulation is applied on the mouse nape of the neck skin using a solvent pipette. When creams are used, they are spread with a finger and allowed to penetrate the skin by massage until complete absorption.

On Day 1, a topical application of acetone/olive oil (4:1 vol:vol) or 0.15% DNFB (2,4 Dinitrofluorobenzene in acetone/olive oil (4:1 vol:vol) is performed on the shaved abdomen under a volume of 100 μL (sensitization phase). On Days 5, 8, 12 and 15, 100 μL of acetone/olive oil or 0.15% DNFB are applied topically on the shaved nape of the neck skin (elicitation phase). From Days 5 to Day 17, BisEDT formulation 1 (group 4), 0% BisEDT formulation 1 (group 3, placebo cream) or 100 mg of 0.05% Clobetasol cream (group 5; control) are applied once daily on the nape of the neck, 2 hours before the DNFB challenge.

Mice are culled on Day 17 under a 3 to 5% isoflurane anesthesia, 4 hours after the last topical treatment, and 48 hours after the last DNFB or acetone/olive oil challenge. Nape of the neck skin samples are collected for histological analyses in formalin solution, 10% neutral buffered. Epidermal and dermal thickness are measured.

### Example 18: Determination of TNF-β Expression in M. Avium-Infected Mice Treated with BisEDT (Pravibismane)

Protocol: M. avium infection was initiated in mice, and 3 weeks were allowed to pass to establish the infection. The animals were treated for 28 days and grouped according to dosage: vehicle, low dose pravibismane, med dose pravibismane, and high dose pravibismane. At the end of treatment, plasma samples were collected for analysis.

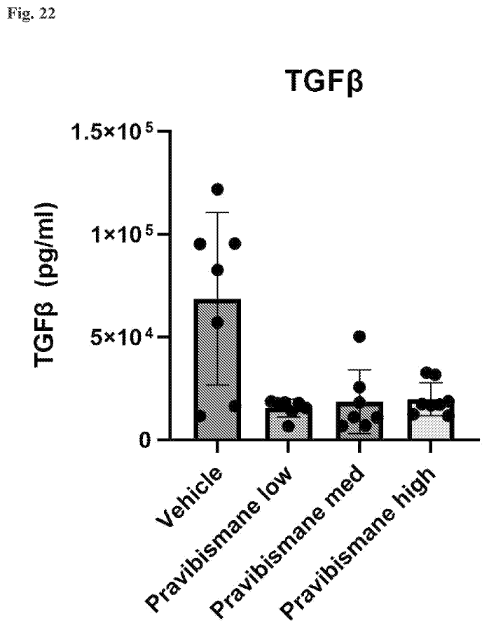

As shown in FIG. 22, there was a consistent reduction in expression of TGF-β in M. avium-infected mice treated with pravibismane compared to vehicle

TGF-β plays a pro-fibrotic role, thus a reduction in TGF-β levels with pravibismane treatment suggests that pravibismane inhibits excessive or exuberant fibrosis.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

What is claimed is:

1. A method of treating an inflammation-related and/or fibrotic disease or condition in a subject in need thereof, comprising administering to the subject an effective amount of a bismuth-thiol (BT) composition comprising pravibismane, wherein the effective amount is effective in the subject to exert an anti-inflammatory effect by therapeutically:
modulating IL-10 levels; and/or,
modulating TNF-α levels; and/or,
inhibiting neutrophil-mediated inflammation/transmigration/infiltration; and/or,
inhibiting excessive or exuberant or pathological fibrosis.

2. The method of claim 1, wherein the inflammation-related disease or condition is an autoimmune disease, a cardiovascular disease, sepsis, a gastrointestinal disorder, a metabolic disease, a liver disease, a neurodegenerative disease, an inflammatory disease of the lung, or an inflammatory condition of the skin.

3. The method of claim 1 or 2, wherein the inflammation-related disease is rheumatoid arthritis (RA), ankylosing spondylitis (AS), or psoriatic arthritis, the cardiovascular disease is heart disease, the inflammatory disease of the lung is chronic obstructive pulmonary disease (COPD) or asthma, the gastrointestinal disorder is ulcerative colitis or Crohn's disease, the metabolic disease is type-2 diabetes, the liver disease is nonalcoholic fatty liver disease (NAFLD) or non-alcoholic steatohepatitis (NASH), the neurodegenerative disease is Parkinson's disease or Alzheimer's disease, the inflammatory disease of the skin is acne vulgaris, atopic dermatitis, or psoriasis.

4. The method of any one of claims 1-3, wherein the inflammation-related disease or condition is an inflammatory disease or condition of the skin.

5. The method of claim 4, wherein the inflammatory disease or condition of the skin is psoriasis or acne vulgaris.

6. The method of any one of claims 1-5, wherein the inflammatory disease or condition of the skin is psoriasis or eczema.

7. The methods of any one of the claims 1-6, wherein the inflammation-related and/or fibrotic disease is an inflammatory arthritide such as arthritis (RA), ankylosing spondylitis (AS), or psoriatic arthritis.

8. The methods of any one of the claims 1-6, wherein the pulmonary inflammatory and/or fibrotic disease is cystic fibrosis (CF), chronic rhinosinusitis, nasal polyps, asthma, non-CF bronchiectasis, chronic obstructive pulmonary disease (COPD), idiopathic pulmonary fibrosis (IPF), sarcoidosis, systemic sclerosis associated interstitial lung disease, pulmonary fibrosis associated with rheumatoid arthritis, and VEXAS Syndrome.

9. The methods of any one of claims 1-8, wherein the gastrointestinal inflammatory and/or fibrotic disease is infectious colitis, Crohn's Disease, Bowel-Associated Dermatosis-Arthritis Syndrome (BADAS), ulcerative colitis, and inflammatory bowel disease (IBD).

10. The methods of any one of claims 1-9, wherein the cardiovascular inflammatory and/or fibrotic disease is cardiac fibrosis, myocarditis, atherosclerosis, and aortic aneurysm.

11. The methods of any one of claims 1-10, wherein the metabolic inflammatory and/or fibrotic disease is diabetes, e.g., type-2 diabetes.

12. The methods of any one of claims 1-11, wherein the liver inflammatory and/or fibrotic disease is a fatty liver disease, e.g., nonalcoholic fatty liver disease (NAFLD) or non-alcoholic steatohepatitis (NASH).

13. The methods of any one of claims 1-12, wherein the neurodegenerative inflammatory and/or fibrotic disease is Parkinson's disease, multiple sclerosis, fibromyalgia, neuro-neutrophilic disease, or Alzheimer's disease or the musculoskeletal inflammatory and/or fibrotic disease is gout and polychrondritis.

14. The method of any one of claims 1 to 13, wherein modulating IL-10 levels comprises increasing IL-10 production in the subject.

15. The method of claim 14 wherein the subject exhibits a reduced IL-10 expression relative to a subject not afflicted with the inflammation-related disease or condition.

16. The method of any one of claims 1 to 15, wherein modulating TNF-α levels comprises decreasing TNF-α production in the subject.

17. The method of any one of claims 1 to 16, wherein the inflammation-related disease or condition is a disease or condition responsive to an increased level of IL-10.

18. The method of any one of claims 1 to 17, wherein the inflammation-related disease or condition is a neutrophil-mediated or neutrophil-dominant disease or condition, wherein the effective amount is effective to reduce migration of neutrophils into a target tissue and thereby inhibit neutrophil-mediated inflammation/transmigration/infiltration.

19. The method of any one of claims 1 to 18, wherein the inflammation-related disease or condition comprises a wound, and the effective amount is effective to mediate wound healing of the wound.

20. The method of any one of claims 1-19, wherein the pravibismane is provided in a composition comprising a plurality of particles of pravibismane suspended therein, wherein the particles have a D90 of less than about 5 μm.

21. The method of any one of claims 1-20, wherein the particles have a D90 of less than about 2 μm and/or the D50 of said particles is less than or equal to about 1.0 μm.

22. The method of any one of claims 1-21, wherein the effective amount is effective to modulate an innate immune activation.

23. The method of any one of the preceding claims, wherein pravibismane is provided in a composition that further comprises about 0.05% to about 1.0% polysorbate 80, optionally about 0.05 to 40 mM sodium chloride, optionally about 1% to about 10% of methylcellulose, and optionally about 2 to 20 mM sodium phosphate at about pH 7.4.

24. The method of any one of the preceding claims, wherein the pravibismane is administered three times per day, two times per day, once daily, every other day, once every three days, three times per week, once every week, once every other week, once every month, or once every other month.

25. The method of any one of the preceding claims, wherein the pravibismane is administered topically or by inhalation.

26. The method of any one of the preceding claims, wherein the pravibismane is administered orally or systemically.

27. The method of any one of claims 1 to 26 treating an inflammation-related disease or condition is regulated by or associated with the activation of an inflammasome.

28. The method of claim 27, wherein the inflammasome is the NLRP3 inflammasome or the AIM2 inflammasome.

29. The method of claim 28, wherein the inflammasome is the NLRP3 inflammasome.

30. The method of any one of claims 27-29, wherein the pravibismane is effective to inhibit expression of the inflammasome.

31. The method of claim 30, wherein the inhibited inflammasome is the NLRP3 inflammasome or the AIM2 inflammasome.

32. The method of any one of claims 1 to 31, wherein the subject is a patient not diagnosed as having a microbial infection.

33. The method of any one of claims 1-31, wherein the effective amount of pravibismane is effective to prevent, reduce, or treat a microbial infection, or to reduce microbial products in the subject.

34. The method of claim 33, wherein the microbial products comprise biofilms and/or superantigens.

35. The method of claim 34, wherein the microbial infection in the subject is at least partially located in the inflammasome-expressing cells of the subject.

36. The method of any one of claims 30-35, wherein the inflammasome-expressing cells are macrophages, white blood cells, bronchial epithelial cells, alveolar epithelial cells, or combinations thereof.

37. The method of claim 36, wherein the inflammasome-expressing cells are macrophages.

38. The method of claim 36 or 37, wherein the macrophages are THP-1 macrophages.

39. The method of any one of claims 35-37, wherein the effective amount of pravibismane is effective to reduce the microbial infection in the inflammasome-expressing cells is reduced.

40. The method of any one of claims 27-39, wherein only cells of the subject that have a microbial infection exhibit increased inflammasome release.

41. The method of claim 40, wherein the release of the NLRP3 inflammasome and/or the AIM2 inflammasome is increased.

42. The method of claim 41, wherein the release of the NLRP3 inflammasome is increased.

43. The method of any one of claims 1-42, wherein the inflammation-related disease or condition is regulated by or associated with the activation of one or more cytokines.

44. The method of claim 43, wherein the one or more cytokines is a pro-inflammatory cytokine selected from one of more IFN-γ, MIP-3α, MIP-1β, TNF-α, IL-22, IL-17A, IL-15, and IL-13.

45. The method of any one of claims 27-44, wherein the administering of pravibismane decreases expression of NLRP3 and/or AIM2 inflammasomes.

46. The method of claim 45, wherein the expression of the NLRP3 and/or AIM2 inflammasomes is decreased by about 50%.

47. The method of any one of claims 1 to 46, wherein the effective amount of pravibismane is effective to inhibit neutrophil-mediated inflammation/transmigration/infiltration.

48. The method of claim 47, wherein the effective amount of pravibismane is effective to reduce the transmigration or recruitment of leukocytes in a target tissue that is a site of inflammation.

49. The method of claim 47, wherein the site of inflammation in the subject has a decreased number of leukocytes after administration of pravibismane to the subject.

50. The method of any one of claims 47-49, wherein the production of IL-10 in the subject is increased after administration of the pravibismane to the subject.

51. The method of any one of claims 47-49, wherein the administration of pravibismane results in a decrease in the secretion of IFN-γ, MIP-3α, MIP-1β, TNF-α, IL-22, IL-17A, IL-15, IL-13, and TGF-β.

52. The method of any one of claims 47-49, wherein the administration of pravibismane results in an increase in the secretion of IL-2 in the cell of a subject.

53. The method of any one of claims 47-49, wherein the leukocytes are neutrophils.

54. The method of any one of claims 1-53, wherein the administration of the pravibismane results in a decrease in the secretion of one or more additional pro-inflammatory cytokines.

55. The method of any one of claims 1-54, wherein the administration of the pravibismane results in a decrease in the secretion of IFN-γ, MIP-3α, MIP-103, TNF-α, IL-22, IL-17A, IL-15, IL-13 and/or TGF-β compared to a subject not administered the pravibismane.

56. The method of any one of claims 1-55, wherein the administration of pravibismane results in a decrease in the secretion of MIP-3α, MIP-1β, and/or TNF-α compared to a subject not administered the pravibismane.

57. The method of any one of claims 1-56, wherein the administration of the pravibismane results in a decrease in the secretion of TNF-α compared to a subject not administered the pravibismane.

58. The method of any one of claims 55-57, wherein the secretion of IFN-γ, MIP-3α, MIP-1β, TNF-α, IL-22, IL-17A, IL-15, and/or IL-13 is decreased by about 2-fold, about 3-fold, about 4-fold, about 5-fold, about 6-fold, about 7-fold, about 8-fold, about 9-fold, or about 10-fold compared to a subject not administered the pravibismane.

59. The method of any one of claims 55-57, wherein the secretion of IFN-γ, MIP-3α, MIP-1β, TNF-α, IL-22, IL-17A, IL-15, and/or IL-13 is decreased by about 3-fold compared to a subject not administered pravibismane.

60. The method of any one of claims 1-59, wherein the administration of the pravibismane to a subject results in an increase in the secretion of one or more additional anti-inflammatory cytokines.

61. The method of any one of claims 1-60, wherein the administration of the pravibismane to a subject results in an increase in the secretion of IL-2 compared to a subject not administered the pravibismane.

62. The method of claim 61, wherein the secretion of IL-2 is increased by about 2-fold, about 3-fold, about 4-fold, about 5-fold, about 6-fold, about 7-fold, about 8-fold, about 9-fold, or about 10-fold compared to a subject not administered the pravibismane.

63. The method of claim 61, wherein the secretion of IL-2 is increased by about 3-fold compared to a subject not administered the pravibismane.

64. The method of any one of claims 1 to 63, wherein the disease or condition is an epithelial tissue disease or condition of an epithelial tissue, optionally wherein the pravibismane is administered to the epithelial tissue.

65. The method of any one of claims 1 to 63, wherein the disease or condition is a dermatological disease or condition of a dermatological tissue, optionally wherein the pravibismane is administered to the dermatological tissue.

66. The method of any one of claims 1 to 63, wherein the disease or condition is a lung disease or condition of a lung tissue, optionally wherein the pravibismane is administered to the lung tissue.
